SEQUENCE LISTING (981 sequences)

SEQ ID NO: 1 - DNA, 88 bp, Artificial Sequence
  Probe
aggaccggatcaacttggagttcagacgtgtgctcttccgatctcacaatttcagtcgtttcttctttggagtcattgcgtgaaccga

SEQ ID NO: 2 - DNA, 86 bp, Artificial Sequence
  Probe
aggaccggatcaacttggagttcagacgtgtgctcttccgatctctattcaaccgggtctgagacaagtttcattgcgtgaaccga

SEQ ID NO: 3 - DNA, 88 bp, Artificial Sequence
  Probe
aggaccggatcaacttggagttcagacgtgtgctcttccgatctagctacattcagcagcattctttttgtctcattgcgtgaaccga

SEQ ID NO: 4 - DNA, 86 bp, Artificial Sequence
  Probe
aggaccggatcaacttggagttcagacgtgtgctcttccgatctgacggctcaaaaccaagagatcgacctcattgcgtgaaccga

SEQ ID NO: 5 - DNA, 85 bp, Artificial Sequence
  Probe
aggaccggatcaacttggagttcagacgtgtgctcttccgatctcgtgcacatggcagaggcagaccacacattgcgtgaaccga

SEQ ID NO: 6 - DNA, 88 bp, Artificial Sequence
  Probe
aggaccggatcaacttggagttcagacgtgtgctcttccgatctacactcctaaagaccgataccaactttttcattgcgtgaaccga

SEQ ID NO: 7 - DNA, 85 bp, Artificial Sequence
  Probe
aggaccggatcaacttggagttcagacgtgtgctcttccgatctgagtgaggtggaagaggaagcccaaacattgcgtgaaccga

SEQ ID NO: 8 - DNA, 86 bp, Artificial Sequence
  Probe
aggaccggatcaacttggagttcagacgtgtgctcttccgatcttactgcttgagtaggagcgtcacatttcattgcgtgaaccga

SEQ ID NO: 9 - DNA, 89 bp, Artificial Sequence
  Probe
aggaccggatcaacttggagttcagacgtgtgctcttccgatctcgtgaattcatgcaatcaagcactttagatcattgcgtgaaccga

SEQ ID NO: 10 - DNA, 87 bp, Artificial Sequence
  Probe
aggaccggatcaacttggagttcagacgtgtgctcttccgatctgagttgaagaaaaatcctgagaacgcctcattgcgtgaaccga

SEQ ID NO: 11 - DNA, 87 bp, Artificial Sequence
  Probe
aggaccggatcaacttggagttcagacgtgtgctcttccgatctctatcacttattatcgttggaccacgaccattgcgtgaaccga

SEQ ID NO: 12 - DNA, 87 bp, Artificial Sequence
  Probe
aggaccggatcaacttggagttcagacgtgtgctcttccgatctatgcacctggatcaaaaagggtcttcaacattgcgtgaaccga

SEQ ID NO: 13 - DNA, 89 bp, Artificial Sequence
  Probe
aggaccggatcaacttggagttcagacgtgtgctcttccgatctctcggtgaattgttgcaggtaaaaaattgtcattgcgtgaaccga

SEQ ID NO: 14 - DNA, 89 bp, Artificial Sequence
  Probe
aggaccggatcaacttggagttcagacgtgtgctcttccgatctctcgaaactgcaatgaaaaatggattggttcattgcgtgaaccga

SEQ ID NO: 15 - DNA, 87 bp, Artificial Sequence
  Probe
aggaccggatcaacttggagttcagacgtgtgctcttccgatcttactggcgaactagtccacaaattcattcattgcgtgaaccga

SEQ ID NO: 16 - DNA, 86 bp, Artificial Sequence
  Probe
aggaccggatcaacttggagttcagacgtgtgctcttccgatctgagtgacgtgacgtgaacaaaccaagacattgcgtgaaccga

SEQ ID NO: 17 - DNA, 84 bp, Artificial Sequence
  Probe
aggaccggatcaacttggagttcagacgtgtgctcttccgatctagctcgtgtggcgtccccctgatttcattgcgtgaaccga

SEQ ID NO: 18 - DNA, 84 bp, Artificial Sequence
  Probe
aggaccggatcaacttggagttcagacgtgtgctcttccgatctgagtttccgggcagctaggagggttcattgcgtgaaccga

SEQ ID NO: 19 - DNA, 89 bp, Artificial Sequence
  Probe
aggaccggatcaacttggagttcagacgtgtgctcttccgatctagtctacttgattgatctaataaagcagcacattgcgtgaaccga

SEQ ID NO: 20 - DNA, 86 bp, Artificial Sequence
  Probe
aggaccggatcaacttggagttcagacgtgtgctcttccgatctcgtggcaccgtaccaatatctctggatcattgcgtgaaccga

SEQ ID NO: 21 - DNA, 89 bp, Artificial Sequence
  Probe
aggaccggatcaacttggagttcagacgtgtgctcttccgatctcgtggtgtgtggtacaaacaaatgaacatacattgcgtgaaccga

SEQ ID NO: 22 - DNA, 85 bp, Artificial Sequence
  Probe
aggaccggatcaacttggagttcagacgtgtgctcttccgatctatgcactgctgcggctgagtgttgaacattgcgtgaaccga

SEQ ID NO: 23 - DNA, 87 bp, Artificial Sequence
  Probe
aggaccggatcaacttggagttcagacgtgtgctcttccgatctcgtgcatagctatgctatggttcgcatacattgcgtgaaccga

SEQ ID NO: 24 - DNA, 88 bp, Artificial Sequence
  Probe
aggaccggatcaacttggagttcagacgtgtgctcttccgatctatcagctatcatcatcagagaaaccatttcattgcgtgaaccga

SEQ ID NO: 25 - DNA, 86 bp, Artificial Sequence
  Probe
aggaccggatcaacttggagttcagacgtgtgctcttccgatctatgcctgcatggctgcatcgctttcaacattgcgtgaaccga

SEQ ID NO: 26 - DNA, 89 bp, Artificial Sequence
  Probe
aggaccggatcaacttggagttcagacgtgtgctcttccgatctgcataccttgcacttttaatcttaactacacattgcgtgaaccga

SEQ ID NO: 27 - DNA, 86 bp, Artificial Sequence
  Probe
aggaccggatcaacttggagttcagacgtgtgctcttccgatctactactggtttggcagacgatcacacacattgcgtgaaccga

SEQ ID NO: 28 - DNA, 86 bp, Artificial Sequence
  Probe
aggaccggatcaacttggagttcagacgtgtgctcttccgatctctatactgtactcacacacagggcaatcattgcgtgaaccga

SEQ ID NO: 29 - DNA, 88 bp, Artificial Sequence
  Probe
aggaccggatcaacttggagttcagacgtgtgctcttccgatctagctgagatttctgaaaacctaagcccatcattgcgtgaaccga

SEQ ID NO: 30 - DNA, 88 bp, Artificial Sequence
  Probe
aggaccggatcaacttggagttcagacgtgtgctcttccgatctcgtgaccaaggataatcttgttccatcttcattgcgtgaaccga

SEQ ID NO: 31 - DNA, 88 bp, Artificial Sequence
  Probe
aggaccggatcaacttggagttcagacgtgtgctcttccgatctatgccagatgaaacttagtatggtgtagtcattgcgtgaaccga

SEQ ID NO: 32 - DNA, 86 bp, Artificial Sequence
  Probe
aggaccggatcaacttggagttcagacgtgtgctcttccgatcttactcggcaagtacagtcatctctcttcattgcgtgaaccga

SEQ ID NO: 33 - DNA, 87 bp, Artificial Sequence
  Probe
aggaccggatcaacttggagttcagacgtgtgctcttccgatctatgctgcaacttggagcatctctacattcattgcgtgaaccga

SEQ ID NO: 34 - DNA, 88 bp, Artificial Sequence
  Probe
aggaccggatcaacttggagttcagacgtgtgctcttccgatctgcatgtagcagcaaccactttatctgatacattgcgtgaaccga

SEQ ID NO: 35 - DNA, 86 bp, Artificial Sequence
  Probe
aggaccggatcaacttggagttcagacgtgtgctcttccgatctactacacatccggcccaaacttctgaacattgcgtgaaccga

SEQ ID NO: 36 - DNA, 87 bp, Artificial Sequence
  Probe
aggaccggatcaacttggagttcagacgtgtgctcttccgatctcgtggaagtctagctaactgtggatttccattgcgtgaaccga

SEQ ID NO: 37 - DNA, 86 bp, Artificial Sequence
  Probe
aggaccggatcaacttggagttcagacgtgtgctcttccgatctgacgtacaagcgtcaaccaaagagcctcattgcgtgaaccga

SEQ ID NO: 38 - DNA, 86 bp, Artificial Sequence
  Probe
aggaccggatcaacttggagttcagacgtgtgctcttccgatctgagtctacgcgtaccaggaaagatagtcattgcgtgaaccga

SEQ ID NO: 39 - DNA, 87 bp, Artificial Sequence
  Probe
aggaccggatcaacttggagttcagacgtgtgctcttccgatctactaaatctcagtcgccagtttctctttcattgcgtgaaccga

SEQ ID NO: 40 - DNA, 88 bp, Artificial Sequence
  Probe
aggaccggatcaacttggagttcagacgtgtgctcttccgatctatgctcagttggcataataacattgacctcattgcgtgaaccga

SEQ ID NO: 41 - DNA, 88 bp, Artificial Sequence
  Probe
aggaccggatcaacttggagttcagacgtgtgctcttccgatcttactggctaatatgtctgctattgacctacattgcgtgaaccga

SEQ ID NO: 42 - DNA, 85 bp, Artificial Sequence
  Probe
aggaccggatcaacttggagttcagacgtgtgctcttccgatctctgaccacgtcaacggtgcgtagtgtcattgcgtgaaccga

SEQ ID NO: 43 - DNA, 86 bp, Artificial Sequence
  Probe
aggaccggatcaacttggagttcagacgtgtgctcttccgatctactatctcagggatcatgtgtgctcatcattgcgtgaaccga

SEQ ID NO: 44 - DNA, 86 bp, Artificial Sequence
  Probe
aggaccggatcaacttggagttcagacgtgtgctcttccgatctgacgctagcaaccacacagacacaggacattgcgtgaaccga

SEQ ID NO: 45 - DNA, 89 bp, Artificial Sequence
  Probe
aggaccggatcaacttggagttcagacgtgtgctcttccgatctcacaatcagaaaaaactatgacagtctctacattgcgtgaaccga

SEQ ID NO: 46 - DNA, 89 bp, Artificial Sequence
  Probe
aggaccggatcaacttggagttcagacgtgtgctcttccgatctctattatctgttgtgaaaaagaaacccaatcattgcgtgaaccga

SEQ ID NO: 47 - DNA, 86 bp, Artificial Sequence
  Probe
aggaccggatcaacttggagttcagacgtgtgctcttccgatctctgagtagcccattgtgcctcttgttacattgcgtgaaccga

SEQ ID NO: 48 - DNA, 86 bp, Artificial Sequence
  Probe
aggaccggatcaacttggagttcagacgtgtgctcttccgatctatgcatcatccccactccaactaccaacattgcgtgaaccga

SEQ ID NO: 49 - DNA, 86 bp, Artificial Sequence
  Probe
aggaccggatcaacttggagttcagacgtgtgctcttccgatctctcgctagatcctatggccaaagaagccattgcgtgaaccga

SEQ ID NO: 50 - DNA, 87 bp, Artificial Sequence
  Probe
aggaccggatcaacttggagttcagacgtgtgctcttccgatctgagtggttgttacaacggagaagaacgacattgcgtgaaccga

SEQ ID NO: 51 - DNA, 85 bp, Artificial Sequence
  Probe
aggaccggatcaacttggagttcagacgtgtgctcttccgatctatcaggccgggacagtagtatcagttcattgcgtgaaccga

SEQ ID NO: 52 - DNA, 87 bp, Artificial Sequence
  Probe
aggaccggatcaacttggagttcagacgtgtgctcttccgatctgagtcggccatttctttcacacaatcgtcattgcgtgaaccga

SEQ ID NO: 53 - DNA, 86 bp, Artificial Sequence
  Probe
aggaccggatcaacttggagttcagacgtgtgctcttccgatctagtccagttcgcaccctgtgtaatacacattgcgtgaaccga

SEQ ID NO: 54 - DNA, 85 bp, Artificial Sequence
  Probe
aggaccggatcaacttggagttcagacgtgtgctcttccgatctcgtggtctagctgcactggctactgtcattgcgtgaaccga

SEQ ID NO: 55 - DNA, 87 bp, Artificial Sequence
  Probe
aggaccggatcaacttggagttcagacgtgtgctcttccgatctcacaggacacgataatcctctttgggtacattgcgtgaaccga

SEQ ID NO: 56 - DNA, 89 bp, Artificial Sequence
  Probe
aggaccggatcaacttggagttcagacgtgtgctcttccgatctcgtggttacatgaaaaggaagcttgtttcacattgcgtgaaccga

SEQ ID NO: 57 - DNA, 86 bp, Artificial Sequence
  Probe
aggaccggatcaacttggagttcagacgtgtgctcttccgatctctcgatggttgctgctcaagtctacgtcattgcgtgaaccga

SEQ ID NO: 58 - DNA, 88 bp, Artificial Sequence
  Probe
aggaccggatcaacttggagttcagacgtgtgctcttccgatcttactcagtgagatgacagtgatatggtttcattgcgtgaaccga

SEQ ID NO: 59 - DNA, 87 bp, Artificial Sequence
  Probe
aggaccggatcaacttggagttcagacgtgtgctcttccgatctagcttgcttaacatggtttctgctgagtcattgcgtgaaccga

SEQ ID NO: 60 - DNA, 87 bp, Artificial Sequence
  Probe
aggaccggatcaacttggagttcagacgtgtgctcttccgatctctcgctcaaactaaccgttggatgaggtcattgcgtgaaccga

SEQ ID NO: 61 - DNA, 87 bp, Artificial Sequence
  Probe
aggaccggatcaacttggagttcagacgtgtgctcttccgatctctatacgttatgaagctgttgcaaggaacattgcgtgaaccga

SEQ ID NO: 62 - DNA, 85 bp, Artificial Sequence
  Probe
aggaccggatcaacttggagttcagacgtgtgctcttccgatctatcacagcagccattcgttccacagtcattgcgtgaaccga

SEQ ID NO: 63 - DNA, 87 bp, Artificial Sequence
  Probe
aggaccggatcaacttggagttcagacgtgtgctcttccgatctagcttagatggagaaattgtaaccggcacattgcgtgaaccga

SEQ ID NO: 64 - DNA, 87 bp, Artificial Sequence
  Probe
aggaccggatcaacttggagttcagacgtgtgctcttccgatctctgagcacacaattgatctgcagtgactcattgcgtgaaccga

SEQ ID NO: 65 - DNA, 87 bp, Artificial Sequence
  Probe
aggaccggatcaacttggagttcagacgtgtgctcttccgatcttactaagtcccacgtggtacataattctcattgcgtgaaccga

SEQ ID NO: 66 - DNA, 87 bp, Artificial Sequence
  Probe
aggaccggatcaacttggagttcagacgtgtgctcttccgatctcacatggtcgttaatcacgagatcaacacattgcgtgaaccga

SEQ ID NO: 67 - DNA, 89 bp, Artificial Sequence
  Probe
aggaccggatcaacttggagttcagacgtgtgctcttccgatctctatctgaaaaacctttggaataagtgcttcattgcgtgaaccga

SEQ ID NO: 68 - DNA, 87 bp, Artificial Sequence
  Probe
aggaccggatcaacttggagttcagacgtgtgctcttccgatctctattttctgacgtctcaactgttccttcattgcgtgaaccga

SEQ ID NO: 69 - DNA, 86 bp, Artificial Sequence
  Probe
aggaccggatcaacttggagttcagacgtgtgctcttccgatctacaccgacttctctagttcctcagtcacattgcgtgaaccga

SEQ ID NO: 70 - DNA, 87 bp, Artificial Sequence
  Probe
aggaccggatcaacttggagttcagacgtgtgctcttccgatcttacttggaatttcttggagaagttccctcattgcgtgaaccga

SEQ ID NO: 71 - DNA, 87 bp, Artificial Sequence
  Probe
aggaccggatcaacttggagttcagacgtgtgctcttccgatctactatggtatttatactgtgagctgagccattgcgtgaaccga

SEQ ID NO: 72 - DNA, 87 bp, Artificial Sequence
  Probe
aggaccggatcaacttggagttcagacgtgtgctcttccgatctatcaagctcaagaggaaaatcagcatctcattgcgtgaaccga

SEQ ID NO: 73 - DNA, 87 bp, Artificial Sequence
  Probe
aggaccggatcaacttggagttcagacgtgtgctcttccgatctctcgtagtatgtgtttgatcgcgctagtcattgcgtgaaccga

SEQ ID NO: 74 - DNA, 88 bp, Artificial Sequence
  Probe
aggaccggatcaacttggagttcagacgtgtgctcttccgatctacactaggtaatttataggcggctgattacattgcgtgaaccga

SEQ ID NO: 75 - DNA, 87 bp, Artificial Sequence
  Probe
aggaccggatcaacttggagttcagacgtgtgctcttccgatctagctgccggctattgcagacaaaaagatcattgcgtgaaccga

SEQ ID NO: 76 - DNA, 86 bp, Artificial Sequence
  Probe
aggaccggatcaacttggagttcagacgtgtgctcttccgatctagtctttgtgggagaggaattctggcacattgcgtgaaccga

SEQ ID NO: 77 - DNA, 86 bp, Artificial Sequence
  Probe
aggaccggatcaacttggagttcagacgtgtgctcttccgatctctcgcctcgtcttctttcacctctccacattgcgtgaaccga

SEQ ID NO: 78 - DNA, 88 bp, Artificial Sequence
  Probe
aggaccggatcaacttggagttcagacgtgtgctcttccgatctagtccagtacaaccttgcagattttggtacattgcgtgaaccga

SEQ ID NO: 79 - DNA, 87 bp, Artificial Sequence
  Probe
aggaccggatcaacttggagttcagacgtgtgctcttccgatctcgtgtagttgtagatctgggggttacttcattgcgtgaaccga

SEQ ID NO: 80 - DNA, 85 bp, Artificial Sequence
  Probe
aggaccggatcaacttggagttcagacgtgtgctcttccgatctgacggctctcactagagcccctacatcattgcgtgaaccga

SEQ ID NO: 81 - DNA, 86 bp, Artificial Sequence
  Probe
aggaccggatcaacttggagttcagacgtgtgctcttccgatctctcggtacggtggttggaacagtaactcattgcgtgaaccga

SEQ ID NO: 82 - DNA, 86 bp, Artificial Sequence
  Probe
aggaccggatcaacttggagttcagacgtgtgctcttccgatctgagtcgtatacacgcacatgtgtgtgtcattgcgtgaaccga

SEQ ID NO: 83 - DNA, 87 bp, Artificial Sequence
  Probe
aggaccggatcaacttggagttcagacgtgtgctcttccgatcttactatgagctgcagtttgcttcttactcattgcgtgaaccga

SEQ ID NO: 84 - DNA, 84 bp, Artificial Sequence
  Probe
aggaccggatcaacttggagttcagacgtgtgctcttccgatctgagtggaccaacttgtcggcgccaacattgcgtgaaccga

SEQ ID NO: 85 - DNA, 87 bp, Artificial Sequence
  Probe
aggaccggatcaacttggagttcagacgtgtgctcttccgatcttactgcatgcggaaaataatggagtactcattgcgtgaaccga

SEQ ID NO: 86 - DNA, 89 bp, Artificial Sequence
  Probe
aggaccggatcaacttggagttcagacgtgtgctcttccgatctacacaaaaacacattctgcaagcaaaacatcattgcgtgaaccga

SEQ ID NO: 87 - DNA, 86 bp, Artificial Sequence
  Probe
aggaccggatcaacttggagttcagacgtgtgctcttccgatctctgatttgaggagggtgctgcaagattcattgcgtgaaccga

SEQ ID NO: 88 - DNA, 86 bp, Artificial Sequence
  Probe
aggaccggatcaacttggagttcagacgtgtgctcttccgatctactagggtgtacattggtttgcttgctcattgcgtgaaccga

SEQ ID NO: 89 - DNA, 86 bp, Artificial Sequence
  Probe
aggaccggatcaacttggagttcagacgtgtgctcttccgatctatgctatcgtgcttctccaggtaacgacattgcgtgaaccga

SEQ ID NO: 90 - DNA, 86 bp, Artificial Sequence
  Probe
aggaccggatcaacttggagttcagacgtgtgctcttccgatctgcatatatggccgatctgggtagtgtacattgcgtgaaccga

SEQ ID NO: 91 - DNA, 86 bp, Artificial Sequence
  Probe
aggaccggatcaacttggagttcagacgtgtgctcttccgatctagctgggtgtctggttcttcaaacagtcattgcgtgaaccga

SEQ ID NO: 92 - DNA, 88 bp, Artificial Sequence
  Probe
aggaccggatcaacttggagttcagacgtgtgctcttccgatctactatgatcgagctgattagtttctagatcattgcgtgaaccga

SEQ ID NO: 93 - DNA, 88 bp, Artificial Sequence
  Probe
aggaccggatcaacttggagttcagacgtgtgctcttccgatctatgccggcttcatgtttctcccaaaaaatcattgcgtgaaccga

SEQ ID NO: 94 - DNA, 87 bp, Artificial Sequence
  Probe
aggaccggatcaacttggagttcagacgtgtgctcttccgatctctcggaagccctctaagttcatcgacttcattgcgtgaaccga

SEQ ID NO: 95 - DNA, 88 bp, Artificial Sequence
  Probe
aggaccggatcaacttggagttcagacgtgtgctcttccgatctgagtgttgaaatgctttctaatggtgggacattgcgtgaaccga

SEQ ID NO: 96 - DNA, 89 bp, Artificial Sequence
  Probe
aggaccggatcaacttggagttcagacgtgtgctcttccgatcttactatacagcaacatcataacacatatgacattgcgtgaaccga

SEQ ID NO: 97 - DNA, 86 bp, Artificial Sequence
  Probe
aggaccggatcaacttggagttcagacgtgtgctcttccgatctctcgctaatcctttgccgtgctcagctcattgcgtgaaccga

SEQ ID NO: 98 - DNA, 87 bp, Artificial Sequence
  Probe
aggaccggatcaacttggagttcagacgtgtgctcttccgatcttactgttttggatcctcaaagagaaggtcattgcgtgaaccga

SEQ ID NO: 99 - DNA, 87 bp, Artificial Sequence
  Probe
aggaccggatcaacttggagttcagacgtgtgctcttccgatctacacgaccctgttgttggctatacagatcattgcgtgaaccga

SEQ ID NO: 100 - DNA, 86 bp, Artificial Sequence
  Probe
aggaccggatcaacttggagttcagacgtgtgctcttccgatctcacaattatcccgggcaagtccatgatcattgcgtgaaccga

SEQ ID NO: 101 - DNA, 85 bp, Artificial Sequence
  Probe
aggaccggatcaacttggagttcagacgtgtgctcttccgatctctatggcaggtgcagacaacggcaaacattgcgtgaaccga

SEQ ID NO: 102 - DNA, 84 bp, Artificial Sequence
  Probe
aggaccggatcaacttggagttcagacgtgtgctcttccgatctatcaccgatcgggcggttgagatcacattgcgtgaaccga

SEQ ID NO: 103 - DNA, 86 bp, Artificial Sequence
  Probe
aggaccggatcaacttggagttcagacgtgtgctcttccgatcttactgttcggtcacggcggttgaatttcattgcgtgaaccga

SEQ ID NO: 104 - DNA, 85 bp, Artificial Sequence
  Probe
aggaccggatcaacttggagttcagacgtgtgctcttccgatctgcatttgcagcagcaacccacggtttcattgcgtgaaccga

SEQ ID NO: 105 - DNA, 89 bp, Artificial Sequence
  Probe
aggaccggatcaacttggagttcagacgtgtgctcttccgatctactagtctagaatgaatttagcagacttgacattgcgtgaaccga

SEQ ID NO: 106 - DNA, 90 bp, Artificial Sequence
  Probe
aggaccggatcaacttggagttcagacgtgtgctcttccgatctacacttcttttcttttacaacagacttacatcattgcgtgaaccga

SEQ ID NO: 107 - DNA, 86 bp, Artificial Sequence
  Probe
aggaccggatcaacttggagttcagacgtgtgctcttccgatctgacggtcctgctggtcagcgtttctaacattgcgtgaaccga

SEQ ID NO: 108 - DNA, 88 bp, Artificial Sequence
  Probe
aggaccggatcaacttggagttcagacgtgtgctcttccgatctcacattaatagcgatgtgtttcagttgcacattgcgtgaaccga

SEQ ID NO: 109 - DNA, 85 bp, Artificial Sequence
  Probe
aggaccggatcaacttggagttcagacgtgtgctcttccgatctctcgttgcagcctccggtcacacaaacattgcgtgaaccga

SEQ ID NO: 110 - DNA, 86 bp, Artificial Sequence
  Probe
aggaccggatcaacttggagttcagacgtgtgctcttccgatctatcacatcgtcacagtcagtagtagctcattgcgtgaaccga

SEQ ID NO: 111 - DNA, 86 bp, Artificial Sequence
  Probe
aggaccggatcaacttggagttcagacgtgtgctcttccgatctagtcgacacgatgatgtggagaaaggtcattgcgtgaaccga

SEQ ID NO: 112 - DNA, 87 bp, Artificial Sequence
  Probe
aggaccggatcaacttggagttcagacgtgtgctcttccgatctacactgcattagattcgccacttaggatcattgcgtgaaccga

SEQ ID NO: 113 - DNA, 86 bp, Artificial Sequence
  Probe
aggaccggatcaacttggagttcagacgtgtgctcttccgatctagctcaggagacagagttctgcacaatcattgcgtgaaccga

SEQ ID NO: 114 - DNA, 88 bp, Artificial Sequence
  Probe
aggaccggatcaacttggagttcagacgtgtgctcttccgatctctatcattagctgagtcaattcagtcctacattgcgtgaaccga

SEQ ID NO: 115 - DNA, 86 bp, Artificial Sequence
  Probe
aggaccggatcaacttggagttcagacgtgtgctcttccgatctacacgacgactaacgtgtcttgcttcacattgcgtgaaccga

SEQ ID NO: 116 - DNA, 87 bp, Artificial Sequence
  Probe
aggaccggatcaacttggagttcagacgtgtgctcttccgatctagctcaaaacaccagtagcatgcactatcattgcgtgaaccga

SEQ ID NO: 117 - DNA, 84 bp, Artificial Sequence
  Probe
aggaccggatcaacttggagttcagacgtgtgctcttccgatctctgaccaaccgatcgagcgagcatccattgcgtgaaccga

SEQ ID NO: 118 - DNA, 87 bp, Artificial Sequence
  Probe
aggaccggatcaacttggagttcagacgtgtgctcttccgatctcacattcacaaaagcatttggcgctacacattgcgtgaaccga

SEQ ID NO: 119 - DNA, 85 bp, Artificial Sequence
  Probe
aggaccggatcaacttggagttcagacgtgtgctcttccgatctatgccagagctgagagcagtggacgtcattgcgtgaaccga

SEQ ID NO: 120 - DNA, 88 bp, Artificial Sequence
  Probe
aggaccggatcaacttggagttcagacgtgtgctcttccgatctctcgctctgaagtccttgtccagtaaaatcattgcgtgaaccga

SEQ ID NO: 121 - DNA, 87 bp, Artificial Sequence
  Probe
aggaccggatcaacttggagttcagacgtgtgctcttccgatctgacggtgacagttgtcaaacagaccaatcattgcgtgaaccga

SEQ ID NO: 122 - DNA, 89 bp, Artificial Sequence
  Probe
aggaccggatcaacttggagttcagacgtgtgctcttccgatctctgatatattaagattgtgtgctgcaagttcattgcgtgaaccga

SEQ ID NO: 123 - DNA, 86 bp, Artificial Sequence
  Probe
aggaccggatcaacttggagttcagacgtgtgctcttccgatctactaaagcggttgcaataaaccagccacattgcgtgaaccga

SEQ ID NO: 124 - DNA, 86 bp, Artificial Sequence
  Probe
aggaccggatcaacttggagttcagacgtgtgctcttccgatctctcgcatcggatgtgcggtcaagaactcattgcgtgaaccga

SEQ ID NO: 125 - DNA, 86 bp, Artificial Sequence
  Probe
aggaccggatcaacttggagttcagacgtgtgctcttccgatctgagtccatactaagctgccactcacttcattgcgtgaaccga

SEQ ID NO: 126 - DNA, 85 bp, Artificial Sequence
  Probe
aggaccggatcaacttggagttcagacgtgtgctcttccgatctctgaggtgtgtcctcatcctcatcgacattgcgtgaaccga

SEQ ID NO: 127 - DNA, 87 bp, Artificial Sequence
  Probe
aggaccggatcaacttggagttcagacgtgtgctcttccgatctctgatttcagactttcagctgcgatgaacattgcgtgaaccga

SEQ ID NO: 128 - DNA, 85 bp, Artificial Sequence
  Probe
aggaccggatcaacttggagttcagacgtgtgctcttccgatctagtcctcatcttcccggtccgaacgacattgcgtgaaccga

SEQ ID NO: 129 - DNA, 86 bp, Artificial Sequence
  Probe
aggaccggatcaacttggagttcagacgtgtgctcttccgatctactacctcagtaccaagacgacgaagacattgcgtgaaccga

SEQ ID NO: 130 - DNA, 85 bp, Artificial Sequence
  Probe
aggaccggatcaacttggagttcagacgtgtgctcttccgatctgcatccgctgcaaaaggatggggcttcattgcgtgaaccga

SEQ ID NO: 131 - DNA, 86 bp, Artificial Sequence
  Probe
aggaccggatcaacttggagttcagacgtgtgctcttccgatctgacgcaaggtggaccagaagagaaactcattgcgtgaaccga

SEQ ID NO: 132 - DNA, 86 bp, Artificial Sequence
  Probe
aggaccggatcaacttggagttcagacgtgtgctcttccgatctctatgcaaagccttcatttgtgcctctcattgcgtgaaccga

SEQ ID NO: 133 - DNA, 86 bp, Artificial Sequence
  Probe
aggaccggatcaacttggagttcagacgtgtgctcttccgatctacaccaaaaccaacgcagggtgtttcacattgcgtgaaccga

SEQ ID NO: 134 - DNA, 86 bp, Artificial Sequence
  Probe
aggaccggatcaacttggagttcagacgtgtgctcttccgatctgcatctggctgctctctggcaaaaaatcattgcgtgaaccga

SEQ ID NO: 135 - DNA, 87 bp, Artificial Sequence
  Probe
aggaccggatcaacttggagttcagacgtgtgctcttccgatctatcacagagtactaccagttgctcgtaacattgcgtgaaccga

SEQ ID NO: 136 - DNA, 87 bp, Artificial Sequence
  Probe
aggaccggatcaacttggagttcagacgtgtgctcttccgatctctatcattgccatgtgatgctgaggaaacattgcgtgaaccga

SEQ ID NO: 137 - DNA, 86 bp, Artificial Sequence
  Probe
aggaccggatcaacttggagttcagacgtgtgctcttccgatctacacaatgcatctgggactgctctgatcattgcgtgaaccga

SEQ ID NO: 138 - DNA, 86 bp, Artificial Sequence
  Probe
aggaccggatcaacttggagttcagacgtgtgctcttccgatctgacgcgcagcgaacagaattctcgatacattgcgtgaaccga

SEQ ID NO: 139 - DNA, 85 bp, Artificial Sequence
  Probe
aggaccggatcaacttggagttcagacgtgtgctcttccgatctctcgctagccgagctagggatcctcacattgcgtgaaccga

SEQ ID NO: 140 - DNA, 86 bp, Artificial Sequence
  Probe
aggaccggatcaacttggagttcagacgtgtgctcttccgatctgcatcctacatcggcatatctaccatccattgcgtgaaccga

SEQ ID NO: 141 - DNA, 87 bp, Artificial Sequence
  Probe
aggaccggatcaacttggagttcagacgtgtgctcttccgatctgagtcaacacagctgcaaaacatgcattcattgcgtgaaccga

SEQ ID NO: 142 - DNA, 87 bp, Artificial Sequence
  Probe
aggaccggatcaacttggagttcagacgtgtgctcttccgatctactaacgtttgctgcatgttttcagactcattgcgtgaaccga

SEQ ID NO: 143 - DNA, 84 bp, Artificial Sequence
  Probe
aggaccggatcaacttggagttcagacgtgtgctcttccgatctcacagtcctctgggatttcggcgctcattgcgtgaaccga

SEQ ID NO: 144 - DNA, 87 bp, Artificial Sequence
  Probe
aggaccggatcaacttggagttcagacgtgtgctcttccgatctatgcctgaccaatggttagctgacatgacattgcgtgaaccga

SEQ ID NO: 145 - DNA, 86 bp, Artificial Sequence
  Probe
aggaccggatcaacttggagttcagacgtgtgctcttccgatctagtctgcccttcgttgtcctgaacatacattgcgtgaaccga

SEQ ID NO: 146 - DNA, 87 bp, Artificial Sequence
  Probe
aggaccggatcaacttggagttcagacgtgtgctcttccgatcttactgaaagaagctactaatgacctgcacattgcgtgaaccga

SEQ ID NO: 147 - DNA, 87 bp, Artificial Sequence
  Probe
aggaccggatcaacttggagttcagacgtgtgctcttccgatctactagaatcagagcatcctgaatacacacattgcgtgaaccga

SEQ ID NO: 148 - DNA, 86 bp, Artificial Sequence
  Probe
aggaccggatcaacttggagttcagacgtgtgctcttccgatctagctgagtcattattctccatcgcccacattgcgtgaaccga

SEQ ID NO: 149 - DNA, 86 bp, Artificial Sequence
  Probe
aggaccggatcaacttggagttcagacgtgtgctcttccgatctgacgtgccctctgacctagctagttatcattgcgtgaaccga

SEQ ID NO: 150 - DNA, 87 bp, Artificial Sequence
  Probe
aggaccggatcaacttggagttcagacgtgtgctcttccgatctacacactattgagcagtcatccgtctatcattgcgtgaaccga

SEQ ID NO: 151 - DNA, 87 bp, Artificial Sequence
  Probe
aggaccggatcaacttggagttcagacgtgtgctcttccgatctctcgttagtgctacagctacacaagtgtcattgcgtgaaccga

SEQ ID NO: 152 - DNA, 85 bp, Artificial Sequence
  Probe
aggaccggatcaacttggagttcagacgtgtgctcttccgatctgcatggtatgctggccgcaggtacaacattgcgtgaaccga

SEQ ID NO: 153 - DNA, 88 bp, Artificial Sequence
  Probe
aggaccggatcaacttggagttcagacgtgtgctcttccgatctctgagtgtgcagaatcctaatatcggttacattgcgtgaaccga

SEQ ID NO: 154 - DNA, 86 bp, Artificial Sequence
  Probe
aggaccggatcaacttggagttcagacgtgtgctcttccgatctagctcaatgttccacctttgctccacacattgcgtgaaccga

SEQ ID NO: 155 - DNA, 87 bp, Artificial Sequence
  Probe
aggaccggatcaacttggagttcagacgtgtgctcttccgatctctgagtctatccatatcttcacctggcacattgcgtgaaccga

SEQ ID NO: 156 - DNA, 86 bp, Artificial Sequence
  Probe
aggaccggatcaacttggagttcagacgtgtgctcttccgatctgagtgccatcgcattgcaagagctagacattgcgtgaaccga

SEQ ID NO: 157 - DNA, 86 bp, Artificial Sequence
  Probe
aggaccggatcaacttggagttcagacgtgtgctcttccgatctctgaggatccaactgtgcaatgtccaacattgcgtgaaccga

SEQ ID NO: 158 - DNA, 87 bp, Artificial Sequence
  Probe
aggaccggatcaacttggagttcagacgtgtgctcttccgatctcacagcatggaaacctagaaaccaacatcattgcgtgaaccga

SEQ ID NO: 159 - DNA, 85 bp, Artificial Sequence
  Probe
aggaccggatcaacttggagttcagacgtgtgctcttccgatctagtcgacacgagatgccgagtctgcacattgcgtgaaccga

SEQ ID NO: 160 - DNA, 86 bp, Artificial Sequence
  Probe
aggaccggatcaacttggagttcagacgtgtgctcttccgatctctatcccggtctgcgctaataaactatcattgcgtgaaccga

SEQ ID NO: 161 - DNA, 88 bp, Artificial Sequence
  Probe
aggaccggatcaacttggagttcagacgtgtgctcttccgatctatcagtgtaataaacttgccttcatctgccattgcgtgaaccga

SEQ ID NO: 162 - DNA, 87 bp, Artificial Sequence
  Probe
aggaccggatcaacttggagttcagacgtgtgctcttccgatctatgcgctgcgtcccacatattagtgtttcattgcgtgaaccga

SEQ ID NO: 163 - DNA, 87 bp, Artificial Sequence
  Probe
aggaccggatcaacttggagttcagacgtgtgctcttccgatctagtcatccggcatatgttaagtattggccattgcgtgaaccga

SEQ ID NO: 164 - DNA, 87 bp, Artificial Sequence
  Probe
aggaccggatcaacttggagttcagacgtgtgctcttccgatctagctgggggcagaaatctaacaatcagacattgcgtgaaccga

SEQ ID NO: 165 - DNA, 87 bp, Artificial Sequence
  Probe
aggaccggatcaacttggagttcagacgtgtgctcttccgatctctattagtttacagtcaaggggtagagtcattgcgtgaaccga

SEQ ID NO: 166 - DNA, 85 bp, Artificial Sequence
  Probe
aggaccggatcaacttggagttcagacgtgtgctcttccgatctagtcccggataccgcgtatagagtgacattgcgtgaaccga

SEQ ID NO: 167 - DNA, 87 bp, Artificial Sequence
  Probe
aggaccggatcaacttggagttcagacgtgtgctcttccgatctatcacccttccccaatattttttctgctcattgcgtgaaccga

SEQ ID NO: 168 - DNA, 86 bp, Artificial Sequence
  Probe
aggaccggatcaacttggagttcagacgtgtgctcttccgatctgagtcagctagcttttcagtccacagtcattgcgtgaaccga

SEQ ID NO: 169 - DNA, 85 bp, Artificial Sequence
  Probe
aggaccggatcaacttggagttcagacgtgtgctcttccgatctgagtcccgaaacttggtcgtcgtagtcattgcgtgaaccga

SEQ ID NO: 170 - DNA, 87 bp, Artificial Sequence
  Probe
aggaccggatcaacttggagttcagacgtgtgctcttccgatctgcatcatcagcttcactggtaccaactacattgcgtgaaccga

SEQ ID NO: 171 - DNA, 85 bp, Artificial Sequence
  Probe
aggaccggatcaacttggagttcagacgtgtgctcttccgatctactagtctatggtggggagcgatccacattgcgtgaaccga

SEQ ID NO: 172 - DNA, 83 bp, Artificial Sequence
  Probe
aggaccggatcaacttggagttcagacgtgtgctcttccgatctatcagcgagcagcggtagggtgcacattgcgtgaaccga

SEQ ID NO: 173 - DNA, 86 bp, Artificial Sequence
  Probe
aggaccggatcaacttggagttcagacgtgtgctcttccgatctctgatgtgctttctagagctggatgcacattgcgtgaaccga

SEQ ID NO: 174 - DNA, 86 bp, Artificial Sequence
  Probe
aggaccggatcaacttggagttcagacgtgtgctcttccgatctactaggatagctctggagatgacatgacattgcgtgaaccga

SEQ ID NO: 175 - DNA, 87 bp, Artificial Sequence
  Probe
aggaccggatcaacttggagttcagacgtgtgctcttccgatctcacatagcgaggtacttaccacgtaattcattgcgtgaaccga

SEQ ID NO: 176 - DNA, 86 bp, Artificial Sequence
  Probe
aggaccggatcaacttggagttcagacgtgtgctcttccgatctgcattacgctgctggatggaaagatgacattgcgtgaaccga

SEQ ID NO: 177 - DNA, 88 bp, Artificial Sequence
  Probe
aggaccggatcaacttggagttcagacgtgtgctcttccgatctagtcttgggaacagtggagtaacaaaatacattgcgtgaaccga

SEQ ID NO: 178 - DNA, 88 bp, Artificial Sequence
  Probe
aggaccggatcaacttggagttcagacgtgtgctcttccgatctcgtgttgtcagaacccagatttactcaaacattgcgtgaaccga

SEQ ID NO: 179 - DNA, 88 bp, Artificial Sequence
  Probe
aggaccggatcaacttggagttcagacgtgtgctcttccgatctctatccagctgaagtttgtttgaggataacattgcgtgaaccga

SEQ ID NO: 180 - DNA, 87 bp, Artificial Sequence
  Probe
aggaccggatcaacttggagttcagacgtgtgctcttccgatctgcattagctgctctcttcagtttcagtacattgcgtgaaccga

SEQ ID NO: 181 - DNA, 88 bp, Artificial Sequence
  Probe
aggaccggatcaacttggagttcagacgtgtgctcttccgatctgcatgaaaatcttgcaaaacgttggacttcattgcgtgaaccga

SEQ ID NO: 182 - DNA, 86 bp, Artificial Sequence
  Probe
aggaccggatcaacttggagttcagacgtgtgctcttccgatctatcaacggtatcctttctgtcactgctcattgcgtgaaccga

SEQ ID NO: 183 - DNA, 87 bp, Artificial Sequence
  Probe
aggaccggatcaacttggagttcagacgtgtgctcttccgatctcacactcatcaagatctttcacagccaacattgcgtgaaccga

SEQ ID NO: 184 - DNA, 86 bp, Artificial Sequence
  Probe
aggaccggatcaacttggagttcagacgtgtgctcttccgatcttactaattggatgggtaagctgctggacattgcgtgaaccga

SEQ ID NO: 185 - DNA, 89 bp, Artificial Sequence
  Probe
aggaccggatcaacttggagttcagacgtgtgctcttccgatctcacagtaactttggacgataatcaagagatcattgcgtgaaccga

SEQ ID NO: 186 - DNA, 85 bp, Artificial Sequence
  Probe
aggaccggatcaacttggagttcagacgtgtgctcttccgatctctgagccaatcgagcatcccttgcgtcattgcgtgaaccga

SEQ ID NO: 187 - DNA, 86 bp, Artificial Sequence
  Probe
aggaccggatcaacttggagttcagacgtgtgctcttccgatctatcaggtagcagaggttccacatgaatcattgcgtgaaccga

SEQ ID NO: 188 - DNA, 86 bp, Artificial Sequence
  Probe
aggaccggatcaacttggagttcagacgtgtgctcttccgatctagtcatctaccacatcacaggaccgaacattgcgtgaaccga

SEQ ID NO: 189 - DNA, 87 bp, Artificial Sequence
  Probe
aggaccggatcaacttggagttcagacgtgtgctcttccgatctgagtcgttcgtcatggttgacctagatacattgcgtgaaccga

SEQ ID NO: 190 - DNA, 86 bp, Artificial Sequence
  Probe
aggaccggatcaacttggagttcagacgtgtgctcttccgatctgcatcgcaagagacaactccatgagctcattgcgtgaaccga

SEQ ID NO: 191 - DNA, 88 bp, Artificial Sequence
  Probe
aggaccggatcaacttggagttcagacgtgtgctcttccgatctacaccaggccggatttcaaaagtttagttcattgcgtgaaccga

SEQ ID NO: 192 - DNA, 87 bp, Artificial Sequence
  Probe
aggaccggatcaacttggagttcagacgtgtgctcttccgatctacacatctcagcacggaaagttctacaacattgcgtgaaccga

SEQ ID NO: 193 - DNA, 88 bp, Artificial Sequence
  Probe
aggaccggatcaacttggagttcagacgtgtgctcttccgatctgagtctctgatttcttccggtttcaatatcattgcgtgaaccga

SEQ ID NO: 194 - DNA, 88 bp, Artificial Sequence
  Probe
aggaccggatcaacttggagttcagacgtgtgctcttccgatctcgtgtctgatgtactgatacctttttccacattgcgtgaaccga

SEQ ID NO: 195 - DNA, 88 bp, Artificial Sequence
  Probe
aggaccggatcaacttggagttcagacgtgtgctcttccgatctctcgattgtgctgaaaacgtgaattctgtcattgcgtgaaccga

SEQ ID NO: 196 - DNA, 84 bp, Artificial Sequence
  Probe
aggaccggatcaacttggagttcagacgtgtgctcttccgatctagctggcccaatcccggcgtctatacattgcgtgaaccga

SEQ ID NO: 197 - DNA, 87 bp, Artificial Sequence
  Probe
aggaccggatcaacttggagttcagacgtgtgctcttccgatctctcgggagtgttgtttccattggtactacattgcgtgaaccga

SEQ ID NO: 198 - DNA, 85 bp, Artificial Sequence
  Probe
aggaccggatcaacttggagttcagacgtgtgctcttccgatctcacaacctgctggatctgctgaagaccattgcgtgaaccga

SEQ ID NO: 199 - DNA, 88 bp, Artificial Sequence
  Probe
aggaccggatcaacttggagttcagacgtgtgctcttccgatctactagttgttacatctcgtttctctttctcattgcgtgaaccga

SEQ ID NO: 200 - DNA, 87 bp, Artificial Sequence
  Probe
aggaccggatcaacttggagttcagacgtgtgctcttccgatctgacgttatccatgtctccaggtgaagtacattgcgtgaaccga

SEQ ID NO: 201 - DNA, 87 bp, Artificial Sequence
  Probe
aggaccggatcaacttggagttcagacgtgtgctcttccgatctatgcggttcaatgctttacctcctctgacattgcgtgaaccga

SEQ ID NO: 202 - DNA, 84 bp, Artificial Sequence
  Probe
aggaccggatcaacttggagttcagacgtgtgctcttccgatctctcgatccagggcatcagcgcctctcattgcgtgaaccga

SEQ ID NO: 203 - DNA, 87 bp, Artificial Sequence
  Probe
aggaccggatcaacttggagttcagacgtgtgctcttccgatctgcatggcaaggtgaagcttcactgaaatcattgcgtgaaccga

SEQ ID NO: 204 - DNA, 84 bp, Artificial Sequence
  Probe
aggaccggatcaacttggagttcagacgtgtgctcttccgatctatcaaacgccagacgacgcgtctctcattgcgtgaaccga

SEQ ID NO: 205 - DNA, 89 bp, Artificial Sequence
  Probe
aggaccggatcaacttggagttcagacgtgtgctcttccgatctctcgaaaaacaccaccaccatttcatttttcattgcgtgaaccga

SEQ ID NO: 206 - DNA, 87 bp, Artificial Sequence
  Probe
aggaccggatcaacttggagttcagacgtgtgctcttccgatctactaatggggaatctctgcatgtaacaacattgcgtgaaccga

SEQ ID NO: 207 - DNA, 87 bp, Artificial Sequence
  Probe
aggaccggatcaacttggagttcagacgtgtgctcttccgatctctcgagcagagccagctaaaagatcaatcattgcgtgaaccga

SEQ ID NO: 208 - DNA, 87 bp, Artificial Sequence
  Probe
aggaccggatcaacttggagttcagacgtgtgctcttccgatctgcatgctttagctgcacaactgctatgacattgcgtgaaccga

SEQ ID NO: 209 - DNA, 87 bp, Artificial Sequence
  Probe
aggaccggatcaacttggagttcagacgtgtgctcttccgatctatcaggtaagctcttgttttgttgctctcattgcgtgaaccga

SEQ ID NO: 210 - DNA, 88 bp, Artificial Sequence
  Probe
aggaccggatcaacttggagttcagacgtgtgctcttccgatctagcttgatgagatgcatacaaaattgcctcattgcgtgaaccga

SEQ ID NO: 211 - DNA, 87 bp, Artificial Sequence
  Probe
aggaccggatcaacttggagttcagacgtgtgctcttccgatctctcgtccaggattgttgttctgctttctcattgcgtgaaccga

SEQ ID NO: 212 - DNA, 87 bp, Artificial Sequence
  Probe
aggaccggatcaacttggagttcagacgtgtgctcttccgatctgacgtagcctgattgacaatgttgtcctcattgcgtgaaccga

SEQ ID NO: 213 - DNA, 86 bp, Artificial Sequence
  Probe
aggaccggatcaacttggagttcagacgtgtgctcttccgatctctatgggcactgatctaacaacctgaacattgcgtgaaccga

SEQ ID NO: 214 - DNA, 86 bp, Artificial Sequence
  Probe
aggaccggatcaacttggagttcagacgtgtgctcttccgatctatgcccgctgctcgtgtctgaattcttcattgcgtgaaccga

SEQ ID NO: 215 - DNA, 86 bp, Artificial Sequence
  Probe
aggaccggatcaacttggagttcagacgtgtgctcttccgatctcgtgcacgatgaaggcagcttcttcaacattgcgtgaaccga

SEQ ID NO: 216 - DNA, 89 bp, Artificial Sequence
  Probe
aggaccggatcaacttggagttcagacgtgtgctcttccgatctagtcgtctcataatttcaaaatcggatgcacattgcgtgaaccga

SEQ ID NO: 217 - DNA, 87 bp, Artificial Sequence
  Probe
aggaccggatcaacttggagttcagacgtgtgctcttccgatctcgtgaattaaggatgtctatcgaccggacattgcgtgaaccga

SEQ ID NO: 218 - DNA, 90 bp, Artificial Sequence
  Probe
aggaccggatcaacttggagttcagacgtgtgctcttccgatctctcggagtacaacaagagaaaaagagaaatacattgcgtgaaccga

SEQ ID NO: 219 - DNA, 88 bp, Artificial Sequence
  Probe
aggaccggatcaacttggagttcagacgtgtgctcttccgatctctgatgtatacattgtcttggggcttattcattgcgtgaaccga

SEQ ID NO: 220 - DNA, 87 bp, Artificial Sequence
  Probe
aggaccggatcaacttggagttcagacgtgtgctcttccgatctgagtcctgcatctttgtcctatcctatacattgcgtgaaccga

SEQ ID NO: 221 - DNA, 86 bp, Artificial Sequence
  Probe
aggaccggatcaacttggagttcagacgtgtgctcttccgatctctgagctgctggaatataattgggggtcattgcgtgaaccga

SEQ ID NO: 222 - DNA, 84 bp, Artificial Sequence
  Probe
aggaccggatcaacttggagttcagacgtgtgctcttccgatctagtcgaagacccggaccggaaggaacattgcgtgaaccga

SEQ ID NO: 223 - DNA, 90 bp, Artificial Sequence
  Probe
aggaccggatcaacttggagttcagacgtgtgctcttccgatctagtcctctgatactttctttcaaaacataaacattgcgtgaaccga

SEQ ID NO: 224 - DNA, 87 bp, Artificial Sequence
  Probe
aggaccggatcaacttggagttcagacgtgtgctcttccgatctacactcgccataaaagttatgccaccatcattgcgtgaaccga

SEQ ID NO: 225 - DNA, 87 bp, Artificial Sequence
  Probe
aggaccggatcaacttggagttcagacgtgtgctcttccgatctatcaggcgagaaaccacaagttaaacgacattgcgtgaaccga

SEQ ID NO: 226 - DNA, 87 bp, Artificial Sequence
  Probe
aggaccggatcaacttggagttcagacgtgtgctcttccgatctctcgagtcagaaccaatgccgtagtaatcattgcgtgaaccga

SEQ ID NO: 227 - DNA, 86 bp, Artificial Sequence
  Probe
aggaccggatcaacttggagttcagacgtgtgctcttccgatctcacatctgctgctgttgatagtgctaccattgcgtgaaccga

SEQ ID NO: 228 - DNA, 89 bp, Artificial Sequence
  Probe
aggaccggatcaacttggagttcagacgtgtgctcttccgatctctcgtagatcagaccaatgttatcaaactacattgcgtgaaccga

SEQ ID NO: 229 - DNA, 87 bp, Artificial Sequence
  Probe
aggaccggatcaacttggagttcagacgtgtgctcttccgatctctatcgattaattaatggcccctcctcacattgcgtgaaccga

SEQ ID NO: 230 - DNA, 87 bp, Artificial Sequence
  Probe
aggaccggatcaacttggagttcagacgtgtgctcttccgatctctgaactttgaaccattggatggagatccattgcgtgaaccga

SEQ ID NO: 231 - DNA, 89 bp, Artificial Sequence
  Probe
aggaccggatcaacttggagttcagacgtgtgctcttccgatctcacaacttaacaccgtaaagtagagataaacattgcgtgaaccga

SEQ ID NO: 232 - DNA, 87 bp, Artificial Sequence
  Probe
aggaccggatcaacttggagttcagacgtgtgctcttccgatctctgacatcaaatgtgaagtcgtcaccatcattgcgtgaaccga

SEQ ID NO: 233 - DNA, 88 bp, Artificial Sequence
  Probe
aggaccggatcaacttggagttcagacgtgtgctcttccgatctatgcctacgagtacatgcatatacagtaacattgcgtgaaccga

SEQ ID NO: 234 - DNA, 87 bp, Artificial Sequence
  Probe
aggaccggatcaacttggagttcagacgtgtgctcttccgatctgacgcatattccttgatgggcttctggacattgcgtgaaccga

SEQ ID NO: 235 - DNA, 85 bp, Artificial Sequence
  Probe
aggaccggatcaacttggagttcagacgtgtgctcttccgatctcgtgtgcagccatctctaccgacactcattgcgtgaaccga

SEQ ID NO: 236 - DNA, 89 bp, Artificial Sequence
  Probe
aggaccggatcaacttggagttcagacgtgtgctcttccgatctatgcctttgtttttggccgtgaaataaaaacattgcgtgaaccga

SEQ ID NO: 237 - DNA, 86 bp, Artificial Sequence
  Probe
aggaccggatcaacttggagttcagacgtgtgctcttccgatctctatccggttagtacgccatagcgaatcattgcgtgaaccga

SEQ ID NO: 238 - DNA, 87 bp, Artificial Sequence
  Probe
aggaccggatcaacttggagttcagacgtgtgctcttccgatctatcagctgtgctgcgcatttctttgtttcattgcgtgaaccga

SEQ ID NO: 239 - DNA, 87 bp, Artificial Sequence
  Probe
aggaccggatcaacttggagttcagacgtgtgctcttccgatctgacgccttctgaaatcgaagtgcgagaacattgcgtgaaccga

SEQ ID NO: 240 - DNA, 85 bp, Artificial Sequence
  Probe
aggaccggatcaacttggagttcagacgtgtgctcttccgatctgcatgccgagccgatcaagatagtgtcattgcgtgaaccga

SEQ ID NO: 241 - DNA, 87 bp, Artificial Sequence
  Probe
aggaccggatcaacttggagttcagacgtgtgctcttccgatctctgagtcggtagatcacaagcatgataacattgcgtgaaccga

SEQ ID NO: 242 - DNA, 87 bp, Artificial Sequence
  Probe
aggaccggatcaacttggagttcagacgtgtgctcttccgatcttactaagaatgtcttccaaactgcctgacattgcgtgaaccga

SEQ ID NO: 243 - DNA, 88 bp, Artificial Sequence
  Probe
aggaccggatcaacttggagttcagacgtgtgctcttccgatctacaccaaggtttttttgtgaaaggagtgacattgcgtgaaccga

SEQ ID NO: 244 - DNA, 88 bp, Artificial Sequence
  Probe
aggaccggatcaacttggagttcagacgtgtgctcttccgatctagcttttgagggaaatgatctagaatggtcattgcgtgaaccga

SEQ ID NO: 245 - DNA, 87 bp, Artificial Sequence
  Probe
aggaccggatcaacttggagttcagacgtgtgctcttccgatctgagttctaatttcagcagcaaactggctcattgcgtgaaccga

SEQ ID NO: 246 - DNA, 86 bp, Artificial Sequence
  Probe
aggaccggatcaacttggagttcagacgtgtgctcttccgatctctcgccgtcgtcgttctgacatgctttcattgcgtgaaccga

SEQ ID NO: 247 - DNA, 88 bp, Artificial Sequence
  Probe
aggaccggatcaacttggagttcagacgtgtgctcttccgatctcacaactttagaaatccgggtcatcttttcattgcgtgaaccga

SEQ ID NO: 248 - DNA, 86 bp, Artificial Sequence
  Probe
aggaccggatcaacttggagttcagacgtgtgctcttccgatcttactcgattgcttacactgttgcagctcattgcgtgaaccga

SEQ ID NO: 249 - DNA, 87 bp, Artificial Sequence
  Probe
aggaccggatcaacttggagttcagacgtgtgctcttccgatctctcgcagcatatagaagaggggaaggatcattgcgtgaaccga

SEQ ID NO: 250 - DNA, 87 bp, Artificial Sequence
  Probe
aggaccggatcaacttggagttcagacgtgtgctcttccgatctcgtgggagatggttggtgagagtcataacattgcgtgaaccga

SEQ ID NO: 251 - DNA, 87 bp, Artificial Sequence
  Probe
aggaccggatcaacttggagttcagacgtgtgctcttccgatctatgctgataagcatgtgcagcaacttgtcattgcgtgaaccga

SEQ ID NO: 252 - DNA, 86 bp, Artificial Sequence
  Probe
aggaccggatcaacttggagttcagacgtgtgctcttccgatctctgacctggacgtagtcgttgtcaacacattgcgtgaaccga

SEQ ID NO: 253 - DNA, 87 bp, Artificial Sequence
  Probe
aggaccggatcaacttggagttcagacgtgtgctcttccgatctagtcacatagagcgggaaaaaaagtggtcattgcgtgaaccga

SEQ ID NO: 254 - DNA, 89 bp, Artificial Sequence
  Probe
aggaccggatcaacttggagttcagacgtgtgctcttccgatctactagttgtaagtgcacaaaaataaagcaacattgcgtgaaccga

SEQ ID NO: 255 - DNA, 88 bp, Artificial Sequence
  Probe
aggaccggatcaacttggagttcagacgtgtgctcttccgatctatcaaccaaattcaagctgcaagttatctcattgcgtgaaccga

SEQ ID NO: 256 - DNA, 87 bp, Artificial Sequence
  Probe
aggaccggatcaacttggagttcagacgtgtgctcttccgatctgcatcacatccgagtgaagagtaaacaacattgcgtgaaccga

SEQ ID NO: 257 - DNA, 87 bp, Artificial Sequence
  Probe
aggaccggatcaacttggagttcagacgtgtgctcttccgatctcacaggtaatccacaaagttaccagcgtcattgcgtgaaccga

SEQ ID NO: 258 - DNA, 87 bp, Artificial Sequence
  Probe
aggaccggatcaacttggagttcagacgtgtgctcttccgatcttactctagcatgcctctgttatctgcaacattgcgtgaaccga

SEQ ID NO: 259 - DNA, 86 bp, Artificial Sequence
  Probe
aggaccggatcaacttggagttcagacgtgtgctcttccgatctacacaaatgtccaaatcccgccggaatcattgcgtgaaccga

SEQ ID NO: 260 - DNA, 85 bp, Artificial Sequence
  Probe
aggaccggatcaacttggagttcagacgtgtgctcttccgatctctgagctggtagcagccatgcatctacattgcgtgaaccga

SEQ ID NO: 261 - DNA, 87 bp, Artificial Sequence
  Probe
aggaccggatcaacttggagttcagacgtgtgctcttccgatctacactggtatgaccaaactaagtcgacacattgcgtgaaccga

SEQ ID NO: 262 - DNA, 87 bp, Artificial Sequence
  Probe
aggaccggatcaacttggagttcagacgtgtgctcttccgatcttactgaaagcaccacaatcaggtcaaatcattgcgtgaaccga

SEQ ID NO: 263 - DNA, 89 bp, Artificial Sequence
  Probe
aggaccggatcaacttggagttcagacgtgtgctcttccgatctctgaatgtgaactgaagtagtttctttgttcattgcgtgaaccga

SEQ ID NO: 264 - DNA, 88 bp, Artificial Sequence
  Probe
aggaccggatcaacttggagttcagacgtgtgctcttccgatctagctctgaaaatgaggcagcactttcattcattgcgtgaaccga

SEQ ID NO: 265 - DNA, 87 bp, Artificial Sequence
  Probe
aggaccggatcaacttggagttcagacgtgtgctcttccgatctcacaatcgtaaaagctatggctgcagaacattgcgtgaaccga

SEQ ID NO: 266 - DNA, 87 bp, Artificial Sequence
  Probe
aggaccggatcaacttggagttcagacgtgtgctcttccgatctagtcttatggacggtgctcacaaaatgacattgcgtgaaccga

SEQ ID NO: 267 - DNA, 86 bp, Artificial Sequence
  Probe
aggaccggatcaacttggagttcagacgtgtgctcttccgatctagcttgccggcaagctgagtaatttgacattgcgtgaaccga

SEQ ID NO: 268 - DNA, 87 bp, Artificial Sequence
  Probe
aggaccggatcaacttggagttcagacgtgtgctcttccgatctcacacagtacagtctcaagcaatcgattcattgcgtgaaccga

SEQ ID NO: 269 - DNA, 87 bp, Artificial Sequence
  Probe
aggaccggatcaacttggagttcagacgtgtgctcttccgatcttactcttaaacatcctagatcggctcttcattgcgtgaaccga

SEQ ID NO: 270 - DNA, 86 bp, Artificial Sequence
  Probe
aggaccggatcaacttggagttcagacgtgtgctcttccgatctacacgttagttgtcttgcgctcatgcacattgcgtgaaccga

SEQ ID NO: 271 - DNA, 86 bp, Artificial Sequence
  Probe
aggaccggatcaacttggagttcagacgtgtgctcttccgatctgcattgtctaggcctcctaagcttactcattgcgtgaaccga

SEQ ID NO: 272 - DNA, 89 bp, Artificial Sequence
  Probe
aggaccggatcaacttggagttcagacgtgtgctcttccgatctactacagcaagctctattacatcaaagaatcattgcgtgaaccga

SEQ ID NO: 273 - DNA, 85 bp, Artificial Sequence
  Probe
aggaccggatcaacttggagttcagacgtgtgctcttccgatctatcagacagcatgcagcatcgttgcacattgcgtgaaccga

SEQ ID NO: 274 - DNA, 86 bp, Artificial Sequence
  Probe
aggaccggatcaacttggagttcagacgtgtgctcttccgatctatgcacacccccttagatgctctatgacattgcgtgaaccga

SEQ ID NO: 275 - DNA, 86 bp, Artificial Sequence
  Probe
aggaccggatcaacttggagttcagacgtgtgctcttccgatcttactctgtagagggcagcaagtttcaacattgcgtgaaccga

SEQ ID NO: 276 - DNA, 89 bp, Artificial Sequence
  Probe
aggaccggatcaacttggagttcagacgtgtgctcttccgatctatgcggacaaaagaaaaaggacacatgaatcattgcgtgaaccga

SEQ ID NO: 277 - DNA, 89 bp, Artificial Sequence
  Probe
aggaccggatcaacttggagttcagacgtgtgctcttccgatctctcgccgtattagtacagtatttcagagtacattgcgtgaaccga

SEQ ID NO: 278 - DNA, 84 bp, Artificial Sequence
  Probe
aggaccggatcaacttggagttcagacgtgtgctcttccgatcttactgcttgggctgcatcgcctgatcattgcgtgaaccga

SEQ ID NO: 279 - DNA, 88 bp, Artificial Sequence
  Probe
aggaccggatcaacttggagttcagacgtgtgctcttccgatctgagtgattttcagctttgcactaactgatcattgcgtgaaccga

SEQ ID NO: 280 - DNA, 90 bp, Artificial Sequence
  Probe
aggaccggatcaacttggagttcagacgtgtgctcttccgatctatgcgcaaagttgatatcttttccaatctttcattgcgtgaaccga

SEQ ID NO: 281 - DNA, 87 bp, Artificial Sequence
  Probe
aggaccggatcaacttggagttcagacgtgtgctcttccgatctctcgcctgatgaaggcaaaagggaaaaacattgcgtgaaccga

SEQ ID NO: 282 - DNA, 87 bp, Artificial Sequence
  Probe
aggaccggatcaacttggagttcagacgtgtgctcttccgatctgcatagcaaacccggatcagtaacaattcattgcgtgaaccga

SEQ ID NO: 283 - DNA, 89 bp, Artificial Sequence
  Probe
aggaccggatcaacttggagttcagacgtgtgctcttccgatctctatatgattgcagttggtttcattttgatcattgcgtgaaccga

SEQ ID NO: 284 - DNA, 86 bp, Artificial Sequence
  Probe
aggaccggatcaacttggagttcagacgtgtgctcttccgatctagtcacgcaatacagcggtcacaacatcattgcgtgaaccga

SEQ ID NO: 285 - DNA, 90 bp, Artificial Sequence
  Probe
aggaccggatcaacttggagttcagacgtgtgctcttccgatctcgtgcaataagattagcataaaatagtcgttcattgcgtgaaccga

SEQ ID NO: 286 - DNA, 89 bp, Artificial Sequence
  Probe
aggaccggatcaacttggagttcagacgtgtgctcttccgatcttactattttcaccaaaattaagcaggacttcattgcgtgaaccga

SEQ ID NO: 287 - DNA, 86 bp, Artificial Sequence
  Probe
aggaccggatcaacttggagttcagacgtgtgctcttccgatctgagttggtggttattcgggcttttgcacattgcgtgaaccga

SEQ ID NO: 288 - DNA, 87 bp, Artificial Sequence
  Probe
aggaccggatcaacttggagttcagacgtgtgctcttccgatctatcaaagtggcattcagatcaacagtcacattgcgtgaaccga

SEQ ID NO: 289 - DNA, 86 bp, Artificial Sequence
  Probe
aggaccggatcaacttggagttcagacgtgtgctcttccgatctcgtggagagagagagagagagagatcacattgcgtgaaccga

SEQ ID NO: 290 - DNA, 86 bp, Artificial Sequence
  Probe
aggaccggatcaacttggagttcagacgtgtgctcttccgatctgacggccagtaactctttcctccctatcattgcgtgaaccga

SEQ ID NO: 291 - DNA, 87 bp, Artificial Sequence
  Probe
aggaccggatcaacttggagttcagacgtgtgctcttccgatctagtctcaaaggagctagatcttcttcgacattgcgtgaaccga

SEQ ID NO: 292 - DNA, 89 bp, Artificial Sequence
  Probe
aggaccggatcaacttggagttcagacgtgtgctcttccgatctcgtgtgttgaactctttgaacacatcattacattgcgtgaaccga

SEQ ID NO: 293 - DNA, 87 bp, Artificial Sequence
  Probe
aggaccggatcaacttggagttcagacgtgtgctcttccgatctctcggaagaacacaaggcagattgatgtcattgcgtgaaccga

SEQ ID NO: 294 - DNA, 87 bp, Artificial Sequence
  Probe
aggaccggatcaacttggagttcagacgtgtgctcttccgatctgacggcaagtttgtatacttcaggggtacattgcgtgaaccga

SEQ ID NO: 295 - DNA, 85 bp, Artificial Sequence
  Probe
aggaccggatcaacttggagttcagacgtgtgctcttccgatctgcatggacgtccggctgctactactacattgcgtgaaccga

SEQ ID NO: 296 - DNA, 88 bp, Artificial Sequence
  Probe
aggaccggatcaacttggagttcagacgtgtgctcttccgatctagctgactgtagttttgtgcatcttgaatcattgcgtgaaccga

SEQ ID NO: 297 - DNA, 91 bp, Artificial Sequence
  Probe
aggaccggatcaacttggagttcagacgtgtgctcttccgatctactaccagttgagttcgtttatttatttataacattgcgtgaaccga

SEQ ID NO: 298 - DNA, 86 bp, Artificial Sequence
  Probe
aggaccggatcaacttggagttcagacgtgtgctcttccgatctcacacaattggtagggaaggggttccacattgcgtgaaccga

SEQ ID NO: 299 - DNA, 86 bp, Artificial Sequence
  Probe
aggaccggatcaacttggagttcagacgtgtgctcttccgatctagctcccagcaccatgaaggttcatcacattgcgtgaaccga

SEQ ID NO: 300 - DNA, 87 bp, Artificial Sequence
  Probe
aggaccggatcaacttggagttcagacgtgtgctcttccgatctctgaagaagcatggccggttatatacttcattgcgtgaaccga

SEQ ID NO: 301 - DNA, 87 bp, Artificial Sequence
  Probe
aggaccggatcaacttggagttcagacgtgtgctcttccgatctcacaatccacagtaatgtaaccactgctcattgcgtgaaccga

SEQ ID NO: 302 - DNA, 87 bp, Artificial Sequence
  Probe
aggaccggatcaacttggagttcagacgtgtgctcttccgatctagctcttcttgtcaaaaatgaggccagtcattgcgtgaaccga

SEQ ID NO: 303 - DNA, 88 bp, Artificial Sequence
  Probe
aggaccggatcaacttggagttcagacgtgtgctcttccgatctctgacgaaaataaccaaactgcacttctacattgcgtgaaccga

SEQ ID NO: 304 - DNA, 90 bp, Artificial Sequence
  Probe
aggaccggatcaacttggagttcagacgtgtgctcttccgatctctgacagaaaaatttaggcagcacaaaaatacattgcgtgaaccga

SEQ ID NO: 305 - DNA, 88 bp, Artificial Sequence
  Probe
aggaccggatcaacttggagttcagacgtgtgctcttccgatctcacatgttggaaaatcggtgtaccatatacattgcgtgaaccga

SEQ ID NO: 306 - DNA, 90 bp, Artificial Sequence
  Probe
aggaccggatcaacttggagttcagacgtgtgctcttccgatctatcaggtttggttcgttatattatatatagtcattgcgtgaaccga

SEQ ID NO: 307 - DNA, 85 bp, Artificial Sequence
  Probe
aggaccggatcaacttggagttcagacgtgtgctcttccgatctctgatggcagccatgtcagctacagtcattgcgtgaaccga

SEQ ID NO: 308 - DNA, 85 bp, Artificial Sequence
  Probe
aggaccggatcaacttggagttcagacgtgtgctcttccgatctatcaccagctctacaccaaggaatcccattgcgtgaaccga

SEQ ID NO: 309 - DNA, 88 bp, Artificial Sequence
  Probe
aggaccggatcaacttggagttcagacgtgtgctcttccgatctcgtgcaacctttgaagagaacgtgcatatcattgcgtgaaccga

SEQ ID NO: 310 - DNA, 87 bp, Artificial Sequence
  Probe
aggaccggatcaacttggagttcagacgtgtgctcttccgatctctgaggcaaggattatctaagctgctatcattgcgtgaaccga

SEQ ID NO: 311 - DNA, 86 bp, Artificial Sequence
  Probe
aggaccggatcaacttggagttcagacgtgtgctcttccgatctcgtgggaccagactaccagagacagatcattgcgtgaaccga

SEQ ID NO: 312 - DNA, 88 bp, Artificial Sequence
  Probe
aggaccggatcaacttggagttcagacgtgtgctcttccgatcttactctgagttctgtttattttggctgctcattgcgtgaaccga

SEQ ID NO: 313 - DNA, 85 bp, Artificial Sequence
  Probe
aggaccggatcaacttggagttcagacgtgtgctcttccgatctacaccgactacgatgcccccattgatcattgcgtgaaccga

SEQ ID NO: 314 - DNA, 88 bp, Artificial Sequence
  Probe
aggaccggatcaacttggagttcagacgtgtgctcttccgatctgagtcatgaaacgacaacacattcacattcattgcgtgaaccga

SEQ ID NO: 315 - DNA, 87 bp, Artificial Sequence
  Probe
aggaccggatcaacttggagttcagacgtgtgctcttccgatctctgagcaattgtgtttggaggcatacaacattgcgtgaaccga

SEQ ID NO: 316 - DNA, 90 bp, Artificial Sequence
  Probe
aggaccggatcaacttggagttcagacgtgtgctcttccgatctagtcagaatgaagatgtgattatgctattaacattgcgtgaaccga

SEQ ID NO: 317 - DNA, 87 bp, Artificial Sequence
  Probe
aggaccggatcaacttggagttcagacgtgtgctcttccgatcttactgccatttttcacatccagtgatctcattgcgtgaaccga

SEQ ID NO: 318 - DNA, 86 bp, Artificial Sequence
  Probe
aggaccggatcaacttggagttcagacgtgtgctcttccgatctagctgcgtaatgagtccttgcagtacacattgcgtgaaccga

SEQ ID NO: 319 - DNA, 88 bp, Artificial Sequence
  Probe
aggaccggatcaacttggagttcagacgtgtgctcttccgatctgcataacaaatgggttatgcagaagtagtcattgcgtgaaccga

SEQ ID NO: 320 - DNA, 89 bp, Artificial Sequence
  Probe
aggaccggatcaacttggagttcagacgtgtgctcttccgatctctgactatatacgcatttgatgtgcatgttcattgcgtgaaccga

SEQ ID NO: 321 - DNA, 84 bp, Artificial Sequence
  Probe
aggaccggatcaacttggagttcagacgtgtgctcttccgatctactaaccgggcttcccaccaaacgacattgcgtgaaccga

SEQ ID NO: 322 - DNA, 87 bp, Artificial Sequence
  Probe
aggaccggatcaacttggagttcagacgtgtgctcttccgatctcgtgtttttaggaaggccagagtacacacattgcgtgaaccga

SEQ ID NO: 323 - DNA, 87 bp, Artificial Sequence
  Probe
aggaccggatcaacttggagttcagacgtgtgctcttccgatcttactcattgtttccacatcctccttagacattgcgtgaaccga

SEQ ID NO: 324 - DNA, 87 bp, Artificial Sequence
  Probe
aggaccggatcaacttggagttcagacgtgtgctcttccgatctatcacccacacactctcttgtcaatattcattgcgtgaaccga

SEQ ID NO: 325 - DNA, 87 bp, Artificial Sequence
  Probe
aggaccggatcaacttggagttcagacgtgtgctcttccgatctacacccaggttcttggatgtttatggctcattgcgtgaaccga

SEQ ID NO: 326 - DNA, 86 bp, Artificial Sequence
  Probe
aggaccggatcaacttggagttcagacgtgtgctcttccgatctagctcagcaccgtgtccctgtatgtatcattgcgtgaaccga

SEQ ID NO: 327 - DNA, 87 bp, Artificial Sequence
  Probe
aggaccggatcaacttggagttcagacgtgtgctcttccgatcttatgtttcagtcgtttcttctttggagccattgcgtgaaccga

SEQ ID NO: 328 - DNA, 85 bp, Artificial Sequence
  Probe
aggaccggatcaacttggagttcagacgtgtgctcttccgatctcgaccaaccgggtctgagacaagttccattgcgtgaaccga

SEQ ID NO: 329 - DNA, 87 bp, Artificial Sequence
  Probe
aggaccggatcaacttggagttcagacgtgtgctcttccgatctgtctcattcagcagcattctttttgtcccattgcgtgaaccga

SEQ ID NO: 330 - DNA, 86 bp, Artificial Sequence
  Probe
aggaccggatcaacttggagttcagacgtgtgctcttccgatcttcaggctcaaaaccaagagatcgaccccattgcgtgaaccga

SEQ ID NO: 331 - DNA, 84 bp, Artificial Sequence
  Probe
aggaccggatcaacttggagttcagacgtgtgctcttccgatctgcgcacatggcagaggcagaccacccattgcgtgaaccga

SEQ ID NO: 332 - DNA, 87 bp, Artificial Sequence
  Probe
aggaccggatcaacttggagttcagacgtgtgctcttccgatctagagcctaaagaccgataccaacttttgcattgcgtgaaccga

SEQ ID NO: 333 - DNA, 85 bp, Artificial Sequence
  Probe
aggaccggatcaacttggagttcagacgtgtgctcttccgatctgtacgaggtggaagaggaagcccaagcattgcgtgaaccga

SEQ ID NO: 334 - DNA, 86 bp, Artificial Sequence
  Probe
aggaccggatcaacttggagttcagacgtgtgctcttccgatcttgtagcttgagtaggagcgtcacattccattgcgtgaaccga

SEQ ID NO: 335 - DNA, 88 bp, Artificial Sequence
  Probe
aggaccggatcaacttggagttcagacgtgtgctcttccgatctgcgcattcatgcaatcaagcactttagagcattgcgtgaaccga

SEQ ID NO: 336 - DNA, 86 bp, Artificial Sequence
  Probe
aggaccggatcaacttggagttcagacgtgtgctcttccgatctgtacgaagaaaaatcctgagaacgccgcattgcgtgaaccga

SEQ ID NO: 337 - DNA, 87 bp, Artificial Sequence
  Probe
aggaccggatcaacttggagttcagacgtgtgctcttccgatctcgaccacttattatcgttggaccacgagcattgcgtgaaccga

SEQ ID NO: 338 - DNA, 86 bp, Artificial Sequence
  Probe
aggaccggatcaacttggagttcagacgtgtgctcttccgatctcagccctggatcaaaaagggtcttcagcattgcgtgaaccga

SEQ ID NO: 339 - DNA, 89 bp, Artificial Sequence
  Probe
aggaccggatcaacttggagttcagacgtgtgctcttccgatcttctcgtgaattgttgcaggtaaaaaattgccattgcgtgaaccga

SEQ ID NO: 340 - DNA, 88 bp, Artificial Sequence
  Probe
aggaccggatcaacttggagttcagacgtgtgctcttccgatcttctcaactgcaatgaaaaatggattggtgcattgcgtgaaccga

SEQ ID NO: 341 - DNA, 87 bp, Artificial Sequence
  Probe
aggaccggatcaacttggagttcagacgtgtgctcttccgatcttgtaggcgaactagtccacaaattcatccattgcgtgaaccga

SEQ ID NO: 342 - DNA, 86 bp, Artificial Sequence
  Probe
aggaccggatcaacttggagttcagacgtgtgctcttccgatctgtacgacgtgacgtgaacaaaccaaggcattgcgtgaaccga

SEQ ID NO: 343 - DNA, 84 bp, Artificial Sequence
  Probe
aggaccggatcaacttggagttcagacgtgtgctcttccgatctgtctcgtgtggcgtccccctgattgcattgcgtgaaccga

SEQ ID NO: 344 - DNA, 83 bp, Artificial Sequence
  Probe
aggaccggatcaacttggagttcagacgtgtgctcttccgatctgtactccgggcagctaggagggtgcattgcgtgaaccga

SEQ ID NO: 345 - DNA, 88 bp, Artificial Sequence
  Probe
aggaccggatcaacttggagttcagacgtgtgctcttccgatctgctgacttgattgatctaataaagcagcgcattgcgtgaaccga

SEQ ID NO: 346 - DNA, 86 bp, Artificial Sequence
  Probe
aggaccggatcaacttggagttcagacgtgtgctcttccgatctgcgcgcaccgtaccaatatctctggaccattgcgtgaaccga

SEQ ID NO: 347 - DNA, 89 bp, Artificial Sequence
  Probe
aggaccggatcaacttggagttcagacgtgtgctcttccgatctgcgcgtgtgtggtacaaacaaatgaacattcattgcgtgaaccga

SEQ ID NO: 348 - DNA, 84 bp, Artificial Sequence
  Probe
aggaccggatcaacttggagttcagacgtgtgctcttccgatctcagcctgctgcggctgagtgttgaccattgcgtgaaccga

SEQ ID NO: 349 - DNA, 87 bp, Artificial Sequence
  Probe
aggaccggatcaacttggagttcagacgtgtgctcttccgatctgcgccatagctatgctatggttcgcatgcattgcgtgaaccga

SEQ ID NO: 350 - DNA, 88 bp, Artificial Sequence
  Probe
aggaccggatcaacttggagttcagacgtgtgctcttccgatcttgcggctatcatcatcagagaaaccattccattgcgtgaaccga

SEQ ID NO: 351 - DNA, 85 bp, Artificial Sequence
  Probe
aggaccggatcaacttggagttcagacgtgtgctcttccgatctcagctgcatggctgcatcgctttcagcattgcgtgaaccga

SEQ ID NO: 352 - DNA, 88 bp, Artificial Sequence
  Probe
aggaccggatcaacttggagttcagacgtgtgctcttccgatctacgtccttgcacttttaatcttaactacccattgcgtgaaccga

SEQ ID NO: 353 - DNA, 85 bp, Artificial Sequence
  Probe
aggaccggatcaacttggagttcagacgtgtgctcttccgatcttgattggtttggcagacgatcacacgcattgcgtgaaccga

SEQ ID NO: 354 - DNA, 85 bp, Artificial Sequence
  Probe
aggaccggatcaacttggagttcagacgtgtgctcttccgatctcgacctgtactcacacacagggcaaccattgcgtgaaccga

SEQ ID NO: 355 - DNA, 87 bp, Artificial Sequence
  Probe
aggaccggatcaacttggagttcagacgtgtgctcttccgatctgtctagatttctgaaaacctaagcccagcattgcgtgaaccga

SEQ ID NO: 356 - DNA, 87 bp, Artificial Sequence
  Probe
aggaccggatcaacttggagttcagacgtgtgctcttccgatctgcgcccaaggataatcttgttccatctgcattgcgtgaaccga

SEQ ID NO: 357 - DNA, 88 bp, Artificial Sequence
  Probe
aggaccggatcaacttggagttcagacgtgtgctcttccgatctcagccagatgaaacttagtatggtgtagccattgcgtgaaccga

SEQ ID NO: 358 - DNA, 86 bp, Artificial Sequence
  Probe
aggaccggatcaacttggagttcagacgtgtgctcttccgatcttgtacggcaagtacagtcatctctctccattgcgtgaaccga

SEQ ID NO: 359 - DNA, 86 bp, Artificial Sequence
  Probe
aggaccggatcaacttggagttcagacgtgtgctcttccgatctcagcgcaacttggagcatctctacatgcattgcgtgaaccga

SEQ ID NO: 360 - DNA, 87 bp, Artificial Sequence
  Probe
aggaccggatcaacttggagttcagacgtgtgctcttccgatctacgttagcagcaaccactttatctgatgcattgcgtgaaccga

SEQ ID NO: 361 - DNA, 85 bp, Artificial Sequence
  Probe
aggaccggatcaacttggagttcagacgtgtgctcttccgatcttgatacatccggcccaaacttctgagcattgcgtgaaccga

SEQ ID NO: 362 - DNA, 87 bp, Artificial Sequence
  Probe
aggaccggatcaacttggagttcagacgtgtgctcttccgatctgcgcgaagtctagctaactgtggatttgcattgcgtgaaccga

SEQ ID NO: 363 - DNA, 85 bp, Artificial Sequence
  Probe
aggaccggatcaacttggagttcagacgtgtgctcttccgatcttcagacaagcgtcaaccaaagagccccattgcgtgaaccga

SEQ ID NO: 364 - DNA, 86 bp, Artificial Sequence
  Probe
aggaccggatcaacttggagttcagacgtgtgctcttccgatctgtacctacgcgtaccaggaaagatagccattgcgtgaaccga

SEQ ID NO: 365 - DNA, 86 bp, Artificial Sequence
  Probe
aggaccggatcaacttggagttcagacgtgtgctcttccgatcttgatatctcagtcgccagtttctcttccattgcgtgaaccga

SEQ ID NO: 366 - DNA, 87 bp, Artificial Sequence
  Probe
aggaccggatcaacttggagttcagacgtgtgctcttccgatctcagccagttggcataataacattgaccccattgcgtgaaccga

SEQ ID NO: 367 - DNA, 87 bp, Artificial Sequence
  Probe
aggaccggatcaacttggagttcagacgtgtgctcttccgatcttgtagctaatatgtctgctattgacctgcattgcgtgaaccga

SEQ ID NO: 368 - DNA, 84 bp, Artificial Sequence
  Probe
aggaccggatcaacttggagttcagacgtgtgctcttccgatcttagacacgtcaacggtgcgtagtgccattgcgtgaaccga

SEQ ID NO: 369 - DNA, 85 bp, Artificial Sequence
  Probe
aggaccggatcaacttggagttcagacgtgtgctcttccgatcttgatctcagggatcatgtgtgctcaccattgcgtgaaccga

SEQ ID NO: 370 - DNA, 85 bp, Artificial Sequence
  Probe
aggaccggatcaacttggagttcagacgtgtgctcttccgatcttcagtagcaaccacacagacacaggccattgcgtgaaccga

SEQ ID NO: 371 - DNA, 88 bp, Artificial Sequence
  Probe
aggaccggatcaacttggagttcagacgtgtgctcttccgatcttatgtcagaaaaaactatgacagtctctccattgcgtgaaccga

SEQ ID NO: 372 - DNA, 88 bp, Artificial Sequence
  Probe
aggaccggatcaacttggagttcagacgtgtgctcttccgatctcgacatctgttgtgaaaaagaaacccaaccattgcgtgaaccga

SEQ ID NO: 373 - DNA, 86 bp, Artificial Sequence
  Probe
aggaccggatcaacttggagttcagacgtgtgctcttccgatcttagagtagcccattgtgcctcttgttgcattgcgtgaaccga

SEQ ID NO: 374 - DNA, 85 bp, Artificial Sequence
  Probe
aggaccggatcaacttggagttcagacgtgtgctcttccgatctcagctcatccccactccaactaccaccattgcgtgaaccga

SEQ ID NO: 375 - DNA, 86 bp, Artificial Sequence
  Probe
aggaccggatcaacttggagttcagacgtgtgctcttccgatcttctcctagatcctatggccaaagaaggcattgcgtgaaccga

SEQ ID NO: 376 - DNA, 86 bp, Artificial Sequence
  Probe
aggaccggatcaacttggagttcagacgtgtgctcttccgatctgtacgttgttacaacggagaagaacggcattgcgtgaaccga

SEQ ID NO: 377 - DNA, 85 bp, Artificial Sequence
  Probe
aggaccggatcaacttggagttcagacgtgtgctcttccgatcttgcgggccgggacagtagtatcagtccattgcgtgaaccga

SEQ ID NO: 378 - DNA, 86 bp, Artificial Sequence
  Probe
aggaccggatcaacttggagttcagacgtgtgctcttccgatctgtacggccatttctttcacacaatcgccattgcgtgaaccga

SEQ ID NO: 379 - DNA, 86 bp, Artificial Sequence
  Probe
aggaccggatcaacttggagttcagacgtgtgctcttccgatctgctgcagttcgcaccctgtgtaatacgcattgcgtgaaccga

SEQ ID NO: 380 - DNA, 85 bp, Artificial Sequence
  Probe
aggaccggatcaacttggagttcagacgtgtgctcttccgatctgcgcgtctagctgcactggctactgccattgcgtgaaccga

SEQ ID NO: 381 - DNA, 86 bp, Artificial Sequence
  Probe
aggaccggatcaacttggagttcagacgtgtgctcttccgatcttatggacacgataatcctctttgggtccattgcgtgaaccga

SEQ ID NO: 382 - DNA, 88 bp, Artificial Sequence
  Probe
aggaccggatcaacttggagttcagacgtgtgctcttccgatctgcgcttacatgaaaaggaagcttgtttcgcattgcgtgaaccga

SEQ ID NO: 383 - DNA, 85 bp, Artificial Sequence
  Probe
aggaccggatcaacttggagttcagacgtgtgctcttccgatcttctctggttgctgctcaagtctacgccattgcgtgaaccga

SEQ ID NO: 384 - DNA, 87 bp, Artificial Sequence
  Probe
aggaccggatcaacttggagttcagacgtgtgctcttccgatcttgtaagtgagatgacagtgatatggttccattgcgtgaaccga

SEQ ID NO: 385 - DNA, 86 bp, Artificial Sequence
  Probe
aggaccggatcaacttggagttcagacgtgtgctcttccgatctgtctgcttaacatggtttctgctgaggcattgcgtgaaccga

SEQ ID NO: 386 - DNA, 86 bp, Artificial Sequence
  Probe
aggaccggatcaacttggagttcagacgtgtgctcttccgatcttctctcaaactaaccgttggatgaggccattgcgtgaaccga

SEQ ID NO: 387 - DNA, 86 bp, Artificial Sequence
  Probe
aggaccggatcaacttggagttcagacgtgtgctcttccgatctcgaccgttatgaagctgttgcaaggagcattgcgtgaaccga

SEQ ID NO: 388 - DNA, 85 bp, Artificial Sequence
  Probe
aggaccggatcaacttggagttcagacgtgtgctcttccgatcttgcgcagcagccattcgttccacagccattgcgtgaaccga

SEQ ID NO: 389 - DNA, 86 bp, Artificial Sequence
  Probe
aggaccggatcaacttggagttcagacgtgtgctcttccgatctgtctagatggagaaattgtaaccggcgcattgcgtgaaccga

SEQ ID NO: 390 - DNA, 86 bp, Artificial Sequence
  Probe
aggaccggatcaacttggagttcagacgtgtgctcttccgatcttagacacacaattgatctgcagtgacgcattgcgtgaaccga

SEQ ID NO: 391 - DNA, 86 bp, Artificial Sequence
  Probe
aggaccggatcaacttggagttcagacgtgtgctcttccgatcttgtaagtcccacgtggtacataattcgcattgcgtgaaccga

SEQ ID NO: 392 - DNA, 86 bp, Artificial Sequence
  Probe
aggaccggatcaacttggagttcagacgtgtgctcttccgatcttatgggtcgttaatcacgagatcaacgcattgcgtgaaccga

SEQ ID NO: 393 - DNA, 88 bp, Artificial Sequence
  Probe
aggaccggatcaacttggagttcagacgtgtgctcttccgatctcgactgaaaaacctttggaataagtgctccattgcgtgaaccga

SEQ ID NO: 394 - DNA, 86 bp, Artificial Sequence
  Probe
aggaccggatcaacttggagttcagacgtgtgctcttccgatctcgacttctgacgtctcaactgttcctgcattgcgtgaaccga

SEQ ID NO: 395 - DNA, 86 bp, Artificial Sequence
  Probe
aggaccggatcaacttggagttcagacgtgtgctcttccgatctagagcgacttctctagttcctcagtcccattgcgtgaaccga

SEQ ID NO: 396 - DNA, 86 bp, Artificial Sequence
  Probe
aggaccggatcaacttggagttcagacgtgtgctcttccgatcttgtaggaatttcttggagaagttcccccattgcgtgaaccga

SEQ ID NO: 397 - DNA, 87 bp, Artificial Sequence
  Probe
aggaccggatcaacttggagttcagacgtgtgctcttccgatcttgattggtatttatactgtgagctgaggcattgcgtgaaccga

SEQ ID NO: 398 - DNA, 86 bp, Artificial Sequence
  Probe
aggaccggatcaacttggagttcagacgtgtgctcttccgatcttgcggctcaagaggaaaatcagcatcccattgcgtgaaccga

SEQ ID NO: 399 - DNA, 86 bp, Artificial Sequence
  Probe
aggaccggatcaacttggagttcagacgtgtgctcttccgatcttctcagtatgtgtttgatcgcgctagccattgcgtgaaccga

SEQ ID NO: 400 - DNA, 87 bp, Artificial Sequence
  Probe
aggaccggatcaacttggagttcagacgtgtgctcttccgatctagagaggtaatttataggcggctgattgcattgcgtgaaccga

SEQ ID NO: 401 - DNA, 86 bp, Artificial Sequence
  Probe
aggaccggatcaacttggagttcagacgtgtgctcttccgatctgtctccggctattgcagacaaaaagagcattgcgtgaaccga

SEQ ID NO: 402 - DNA, 85 bp, Artificial Sequence
  Probe
aggaccggatcaacttggagttcagacgtgtgctcttccgatctgctgttgtgggagaggaattctggcgcattgcgtgaaccga

SEQ ID NO: 403 - DNA, 85 bp, Artificial Sequence
  Probe
aggaccggatcaacttggagttcagacgtgtgctcttccgatcttctcctcgtcttctttcacctctccgcattgcgtgaaccga

SEQ ID NO: 404 - DNA, 87 bp, Artificial Sequence
  Probe
aggaccggatcaacttggagttcagacgtgtgctcttccgatctgctgagtacaaccttgcagattttggtgcattgcgtgaaccga

SEQ ID NO: 405 - DNA, 86 bp, Artificial Sequence
  Probe
aggaccggatcaacttggagttcagacgtgtgctcttccgatctgcgcagttgtagatctgggggttactccattgcgtgaaccga

SEQ ID NO: 406 - DNA, 85 bp, Artificial Sequence
  Probe
aggaccggatcaacttggagttcagacgtgtgctcttccgatcttcaggctctcactagagcccctacaccattgcgtgaaccga

SEQ ID NO: 407 - DNA, 86 bp, Artificial Sequence
  Probe
aggaccggatcaacttggagttcagacgtgtgctcttccgatcttctcgtacggtggttggaacagtaacccattgcgtgaaccga

SEQ ID NO: 408 - DNA, 86 bp, Artificial Sequence
  Probe
aggaccggatcaacttggagttcagacgtgtgctcttccgatctgtaccgtatacacgcacatgtgtgtgccattgcgtgaaccga

SEQ ID NO: 409 - DNA, 86 bp, Artificial Sequence
  Probe
aggaccggatcaacttggagttcagacgtgtgctcttccgatcttgtatgagctgcagtttgcttcttacgcattgcgtgaaccga

SEQ ID NO: 410 - DNA, 84 bp, Artificial Sequence
  Probe
aggaccggatcaacttggagttcagacgtgtgctcttccgatctgtacggaccaacttgtcggcgccagcattgcgtgaaccga

SEQ ID NO: 411 - DNA, 87 bp, Artificial Sequence
  Probe
aggaccggatcaacttggagttcagacgtgtgctcttccgatcttgtagcatgcggaaaataatggagtacccattgcgtgaaccga

SEQ ID NO: 412 - DNA, 88 bp, Artificial Sequence
  Probe
aggaccggatcaacttggagttcagacgtgtgctcttccgatctagagaaaacacattctgcaagcaaaacaccattgcgtgaaccga

SEQ ID NO: 413 - DNA, 85 bp, Artificial Sequence
  Probe
aggaccggatcaacttggagttcagacgtgtgctcttccgatcttagattgaggagggtgctgcaagatccattgcgtgaaccga

SEQ ID NO: 414 - DNA, 86 bp, Artificial Sequence
  Probe
aggaccggatcaacttggagttcagacgtgtgctcttccgatcttgatgggtgtacattggtttgcttgcccattgcgtgaaccga

SEQ ID NO: 415 - DNA, 85 bp, Artificial Sequence
  Probe
aggaccggatcaacttggagttcagacgtgtgctcttccgatctcagcatcgtgcttctccaggtaacggcattgcgtgaaccga

SEQ ID NO: 416 - DNA, 85 bp, Artificial Sequence
  Probe
aggaccggatcaacttggagttcagacgtgtgctcttccgatctacgttatggccgatctgggtagtgtgcattgcgtgaaccga

SEQ ID NO: 417 - DNA, 86 bp, Artificial Sequence
  Probe
aggaccggatcaacttggagttcagacgtgtgctcttccgatctgtctgggtgtctggttcttcaaacagccattgcgtgaaccga

SEQ ID NO: 418 - DNA, 87 bp, Artificial Sequence
  Probe
aggaccggatcaacttggagttcagacgtgtgctcttccgatcttgatgatcgagctgattagtttctagagcattgcgtgaaccga

SEQ ID NO: 419 - DNA, 87 bp, Artificial Sequence
  Probe
aggaccggatcaacttggagttcagacgtgtgctcttccgatctcagcggcttcatgtttctcccaaaaaagcattgcgtgaaccga

SEQ ID NO: 420 - DNA, 86 bp, Artificial Sequence
  Probe
aggaccggatcaacttggagttcagacgtgtgctcttccgatcttctcaagccctctaagttcatcgactccattgcgtgaaccga

SEQ ID NO: 421 - DNA, 87 bp, Artificial Sequence
  Probe
aggaccggatcaacttggagttcagacgtgtgctcttccgatctgtacttgaaatgctttctaatggtggggcattgcgtgaaccga

SEQ ID NO: 422 - DNA, 88 bp, Artificial Sequence
  Probe
aggaccggatcaacttggagttcagacgtgtgctcttccgatcttgtatacagcaacatcataacacatatgccattgcgtgaaccga

SEQ ID NO: 423 - DNA, 85 bp, Artificial Sequence
  Probe
aggaccggatcaacttggagttcagacgtgtgctcttccgatcttctctaatcctttgccgtgctcagcccattgcgtgaaccga

SEQ ID NO: 424 - DNA, 87 bp, Artificial Sequence
  Probe
aggaccggatcaacttggagttcagacgtgtgctcttccgatcttgtagttttggatcctcaaagagaaggccattgcgtgaaccga

SEQ ID NO: 425 - DNA, 86 bp, Artificial Sequence
  Probe
aggaccggatcaacttggagttcagacgtgtgctcttccgatctagagaccctgttgttggctatacagaccattgcgtgaaccga

SEQ ID NO: 426 - DNA, 85 bp, Artificial Sequence
  Probe
aggaccggatcaacttggagttcagacgtgtgctcttccgatcttatgttatcccgggcaagtccatgaccattgcgtgaaccga

SEQ ID NO: 427 - DNA, 84 bp, Artificial Sequence
  Probe
aggaccggatcaacttggagttcagacgtgtgctcttccgatctcgacgcaggtgcagacaacggcaagcattgcgtgaaccga

SEQ ID NO: 428 - DNA, 84 bp, Artificial Sequence
  Probe
aggaccggatcaacttggagttcagacgtgtgctcttccgatcttgcgccgatcgggcggttgagatcccattgcgtgaaccga

SEQ ID NO: 429 - DNA, 85 bp, Artificial Sequence
  Probe
aggaccggatcaacttggagttcagacgtgtgctcttccgatcttgtattcggtcacggcggttgaattgcattgcgtgaaccga

SEQ ID NO: 430 - DNA, 84 bp, Artificial Sequence
  Probe
aggaccggatcaacttggagttcagacgtgtgctcttccgatctacgttgcagcagcaacccacggttccattgcgtgaaccga

SEQ ID NO: 431 - DNA, 88 bp, Artificial Sequence
  Probe
aggaccggatcaacttggagttcagacgtgtgctcttccgatcttgattctagaatgaatttagcagacttggcattgcgtgaaccga

SEQ ID NO: 432 - DNA, 89 bp, Artificial Sequence
  Probe
aggaccggatcaacttggagttcagacgtgtgctcttccgatctagagtcttttcttttacaacagacttacagcattgcgtgaaccga

SEQ ID NO: 433 - DNA, 85 bp, Artificial Sequence
  Probe
aggaccggatcaacttggagttcagacgtgtgctcttccgatcttcagtcctgctggtcagcgtttctaccattgcgtgaaccga

SEQ ID NO: 434 - DNA, 87 bp, Artificial Sequence
  Probe
aggaccggatcaacttggagttcagacgtgtgctcttccgatcttatgtaatagcgatgtgtttcagttgcgcattgcgtgaaccga

SEQ ID NO: 435 - DNA, 84 bp, Artificial Sequence
  Probe
aggaccggatcaacttggagttcagacgtgtgctcttccgatcttctctgcagcctccggtcacacaagcattgcgtgaaccga

SEQ ID NO: 436 - DNA, 86 bp, Artificial Sequence
  Probe
aggaccggatcaacttggagttcagacgtgtgctcttccgatcttgcgcatcgtcacagtcagtagtagcccattgcgtgaaccga

SEQ ID NO: 437 - DNA, 86 bp, Artificial Sequence
  Probe
aggaccggatcaacttggagttcagacgtgtgctcttccgatctgctggacacgatgatgtggagaaagggcattgcgtgaaccga

SEQ ID NO: 438 - DNA, 86 bp, Artificial Sequence
  Probe
aggaccggatcaacttggagttcagacgtgtgctcttccgatctagaggcattagattcgccacttaggaccattgcgtgaaccga

SEQ ID NO: 439 - DNA, 86 bp, Artificial Sequence
  Probe
aggaccggatcaacttggagttcagacgtgtgctcttccgatctgtctcaggagacagagttctgcacaaccattgcgtgaaccga

SEQ ID NO: 440 - DNA, 87 bp, Artificial Sequence
  Probe
aggaccggatcaacttggagttcagacgtgtgctcttccgatctcgacattagctgagtcaattcagtcctgcattgcgtgaaccga

SEQ ID NO: 441 - DNA, 86 bp, Artificial Sequence
  Probe
aggaccggatcaacttggagttcagacgtgtgctcttccgatctagaggacgactaacgtgtcttgcttcccattgcgtgaaccga

SEQ ID NO: 442 - DNA, 87 bp, Artificial Sequence
  Probe
aggaccggatcaacttggagttcagacgtgtgctcttccgatctgtctcaaaacaccagtagcatgcactaccattgcgtgaaccga

SEQ ID NO: 443 - DNA, 84 bp, Artificial Sequence
  Probe
aggaccggatcaacttggagttcagacgtgtgctcttccgatcttagaccaaccgatcgagcgagcatgcattgcgtgaaccga

SEQ ID NO: 444 - DNA, 86 bp, Artificial Sequence
  Probe
aggaccggatcaacttggagttcagacgtgtgctcttccgatcttatgtcacaaaagcatttggcgctacccattgcgtgaaccga

SEQ ID NO: 445 - DNA, 84 bp, Artificial Sequence
  Probe
aggaccggatcaacttggagttcagacgtgtgctcttccgatctcagcagagctgagagcagtggacgccattgcgtgaaccga

SEQ ID NO: 446 - DNA, 87 bp, Artificial Sequence
  Probe
aggaccggatcaacttggagttcagacgtgtgctcttccgatcttctctctgaagtccttgtccagtaaaaccattgcgtgaaccga

SEQ ID NO: 447 - DNA, 87 bp, Artificial Sequence
  Probe
aggaccggatcaacttggagttcagacgtgtgctcttccgatcttcaggtgacagttgtcaaacagaccaaccattgcgtgaaccga

SEQ ID NO: 448 - DNA, 88 bp, Artificial Sequence
  Probe
aggaccggatcaacttggagttcagacgtgtgctcttccgatcttagaatattaagattgtgtgctgcaagtccattgcgtgaaccga

SEQ ID NO: 449 - DNA, 85 bp, Artificial Sequence
  Probe
aggaccggatcaacttggagttcagacgtgtgctcttccgatcttgatagcggttgcaataaaccagccgcattgcgtgaaccga

SEQ ID NO: 450 - DNA, 85 bp, Artificial Sequence
  Probe
aggaccggatcaacttggagttcagacgtgtgctcttccgatcttctcatcggatgtgcggtcaagaacccattgcgtgaaccga

SEQ ID NO: 451 - DNA, 86 bp, Artificial Sequence
  Probe
aggaccggatcaacttggagttcagacgtgtgctcttccgatctgtacccatactaagctgccactcactccattgcgtgaaccga

SEQ ID NO: 452 - DNA, 85 bp, Artificial Sequence
  Probe
aggaccggatcaacttggagttcagacgtgtgctcttccgatcttagaggtgtgtcctcatcctcatcggcattgcgtgaaccga

SEQ ID NO: 453 - DNA, 86 bp, Artificial Sequence
  Probe
aggaccggatcaacttggagttcagacgtgtgctcttccgatcttagattcagactttcagctgcgatgagcattgcgtgaaccga

SEQ ID NO: 454 - DNA, 84 bp, Artificial Sequence
  Probe
aggaccggatcaacttggagttcagacgtgtgctcttccgatctgctgtcatcttcccggtccgaacggcattgcgtgaaccga

SEQ ID NO: 455 - DNA, 86 bp, Artificial Sequence
  Probe
aggaccggatcaacttggagttcagacgtgtgctcttccgatcttgatcctcagtaccaagacgacgaagtcattgcgtgaaccga

SEQ ID NO: 456 - DNA, 84 bp, Artificial Sequence
  Probe
aggaccggatcaacttggagttcagacgtgtgctcttccgatctacgtcgctgcaaaaggatggggctccattgcgtgaaccga

SEQ ID NO: 457 - DNA, 86 bp, Artificial Sequence
  Probe
aggaccggatcaacttggagttcagacgtgtgctcttccgatcttcagcaaggtggaccagaagagaaacccattgcgtgaaccga

SEQ ID NO: 458 - DNA, 86 bp, Artificial Sequence
  Probe
aggaccggatcaacttggagttcagacgtgtgctcttccgatctcgacgcaaagccttcatttgtgcctcccattgcgtgaaccga

SEQ ID NO: 459 - DNA, 86 bp, Artificial Sequence
  Probe
aggaccggatcaacttggagttcagacgtgtgctcttccgatctagagcaaaaccaacgcagggtgtttcgcattgcgtgaaccga

SEQ ID NO: 460 - DNA, 86 bp, Artificial Sequence
  Probe
aggaccggatcaacttggagttcagacgtgtgctcttccgatctacgtctggctgctctctggcaaaaaaccattgcgtgaaccga

SEQ ID NO: 461 - DNA, 87 bp, Artificial Sequence
  Probe
aggaccggatcaacttggagttcagacgtgtgctcttccgatcttgcgcagagtactaccagttgctcgtatcattgcgtgaaccga

SEQ ID NO: 462 - DNA, 86 bp, Artificial Sequence
  Probe
aggaccggatcaacttggagttcagacgtgtgctcttccgatctcgacattgccatgtgatgctgaggaagcattgcgtgaaccga

SEQ ID NO: 463 - DNA, 85 bp, Artificial Sequence
  Probe
aggaccggatcaacttggagttcagacgtgtgctcttccgatctagagatgcatctgggactgctctgaccattgcgtgaaccga

SEQ ID NO: 464 - DNA, 86 bp, Artificial Sequence
  Probe
aggaccggatcaacttggagttcagacgtgtgctcttccgatcttcagcgcagcgaacagaattctcgatccattgcgtgaaccga

SEQ ID NO: 465 - DNA, 84 bp, Artificial Sequence
  Probe
aggaccggatcaacttggagttcagacgtgtgctcttccgatcttctctagccgagctagggatcctcgcattgcgtgaaccga

SEQ ID NO: 466 - DNA, 86 bp, Artificial Sequence
  Probe
aggaccggatcaacttggagttcagacgtgtgctcttccgatctacgtcctacatcggcatatctaccatgcattgcgtgaaccga

SEQ ID NO: 467 - DNA, 87 bp, Artificial Sequence
  Probe
aggaccggatcaacttggagttcagacgtgtgctcttccgatctgtaccaacacagctgcaaaacatgcatccattgcgtgaaccga

SEQ ID NO: 468 - DNA, 86 bp, Artificial Sequence
  Probe
aggaccggatcaacttggagttcagacgtgtgctcttccgatcttgatcgtttgctgcatgttttcagacgcattgcgtgaaccga

SEQ ID NO: 469 - DNA, 84 bp, Artificial Sequence
  Probe
aggaccggatcaacttggagttcagacgtgtgctcttccgatcttatggtcctctgggatttcggcgcccattgcgtgaaccga

SEQ ID NO: 470 - DNA, 86 bp, Artificial Sequence
  Probe
aggaccggatcaacttggagttcagacgtgtgctcttccgatctcagctgaccaatggttagctgacatggcattgcgtgaaccga

SEQ ID NO: 471 - DNA, 85 bp, Artificial Sequence
  Probe
aggaccggatcaacttggagttcagacgtgtgctcttccgatctgctggcccttcgttgtcctgaacatgcattgcgtgaaccga

SEQ ID NO: 472 - DNA, 87 bp, Artificial Sequence
  Probe
aggaccggatcaacttggagttcagacgtgtgctcttccgatcttgtagaaagaagctactaatgacctgcgcattgcgtgaaccga

SEQ ID NO: 473 - DNA, 87 bp, Artificial Sequence
  Probe
aggaccggatcaacttggagttcagacgtgtgctcttccgatcttgatgaatcagagcatcctgaatacacgcattgcgtgaaccga

SEQ ID NO: 474 - DNA, 86 bp, Artificial Sequence
  Probe
aggaccggatcaacttggagttcagacgtgtgctcttccgatctgtctgagtcattattctccatcgcccccattgcgtgaaccga

SEQ ID NO: 475 - DNA, 85 bp, Artificial Sequence
  Probe
aggaccggatcaacttggagttcagacgtgtgctcttccgatcttcaggccctctgacctagctagttaccattgcgtgaaccga

SEQ ID NO: 476 - DNA, 86 bp, Artificial Sequence
  Probe
aggaccggatcaacttggagttcagacgtgtgctcttccgatctagagctattgagcagtcatccgtctaccattgcgtgaaccga

SEQ ID NO: 477 - DNA, 86 bp, Artificial Sequence
  Probe
aggaccggatcaacttggagttcagacgtgtgctcttccgatcttctctagtgctacagctacacaagtggcattgcgtgaaccga

SEQ ID NO: 478 - DNA, 84 bp, Artificial Sequence
  Probe
aggaccggatcaacttggagttcagacgtgtgctcttccgatctacgtgtatgctggccgcaggtacagcattgcgtgaaccga

SEQ ID NO: 479 - DNA, 87 bp, Artificial Sequence
  Probe
aggaccggatcaacttggagttcagacgtgtgctcttccgatcttagatgtgcagaatcctaatatcggttgcattgcgtgaaccga

SEQ ID NO: 480 - DNA, 86 bp, Artificial Sequence
  Probe
aggaccggatcaacttggagttcagacgtgtgctcttccgatctgtctcaatgttccacctttgctccacccattgcgtgaaccga

SEQ ID NO: 481 - DNA, 86 bp, Artificial Sequence
  Probe
aggaccggatcaacttggagttcagacgtgtgctcttccgatcttagatctatccatatcttcacctggcgcattgcgtgaaccga

SEQ ID NO: 482 - DNA, 85 bp, Artificial Sequence
  Probe
aggaccggatcaacttggagttcagacgtgtgctcttccgatctgtacccatcgcattgcaagagctaggcattgcgtgaaccga

SEQ ID NO: 483 - DNA, 86 bp, Artificial Sequence
  Probe
aggaccggatcaacttggagttcagacgtgtgctcttccgatcttagaggatccaactgtgcaatgtccagcattgcgtgaaccga

SEQ ID NO: 484 - DNA, 87 bp, Artificial Sequence
  Probe
aggaccggatcaacttggagttcagacgtgtgctcttccgatcttatggcatggaaacctagaaaccaacaccattgcgtgaaccga

SEQ ID NO: 485 - DNA, 84 bp, Artificial Sequence
  Probe
aggaccggatcaacttggagttcagacgtgtgctcttccgatctgctgacacgagatgccgagtctgcgcattgcgtgaaccga

SEQ ID NO: 486 - DNA, 86 bp, Artificial Sequence
  Probe
aggaccggatcaacttggagttcagacgtgtgctcttccgatctcgaccccggtctgcgctaataaactaccattgcgtgaaccga

SEQ ID NO: 487 - DNA, 88 bp, Artificial Sequence
  Probe
aggaccggatcaacttggagttcagacgtgtgctcttccgatcttgcggtgtaataaacttgccttcatctggcattgcgtgaaccga

SEQ ID NO: 488 - DNA, 86 bp, Artificial Sequence
  Probe
aggaccggatcaacttggagttcagacgtgtgctcttccgatctcagcctgcgtcccacatattagtgttgcattgcgtgaaccga

SEQ ID NO: 489 - DNA, 87 bp, Artificial Sequence
  Probe
aggaccggatcaacttggagttcagacgtgtgctcttccgatctgctgatccggcatatgttaagtattgggcattgcgtgaaccga

SEQ ID NO: 490 - DNA, 86 bp, Artificial Sequence
  Probe
aggaccggatcaacttggagttcagacgtgtgctcttccgatctgtctggggcagaaatctaacaatcagccattgcgtgaaccga

SEQ ID NO: 491 - DNA, 86 bp, Artificial Sequence
  Probe
aggaccggatcaacttggagttcagacgtgtgctcttccgatctcgacagtttacagtcaaggggtagagccattgcgtgaaccga

SEQ ID NO: 492 - DNA, 85 bp, Artificial Sequence
  Probe
aggaccggatcaacttggagttcagacgtgtgctcttccgatctgctgccggataccgcgtatagagtggcattgcgtgaaccga

SEQ ID NO: 493 - DNA, 87 bp, Artificial Sequence
  Probe
aggaccggatcaacttggagttcagacgtgtgctcttccgatcttgcgcccttccccaatattttttctgcccattgcgtgaaccga

SEQ ID NO: 494 - DNA, 86 bp, Artificial Sequence
  Probe
aggaccggatcaacttggagttcagacgtgtgctcttccgatctgtaccagctagcttttcagtccacagccattgcgtgaaccga

SEQ ID NO: 495 - DNA, 85 bp, Artificial Sequence
  Probe
aggaccggatcaacttggagttcagacgtgtgctcttccgatctgtaccccgaaacttggtcgtcgtaggcattgcgtgaaccga

SEQ ID NO: 496 - DNA, 86 bp, Artificial Sequence
  Probe
aggaccggatcaacttggagttcagacgtgtgctcttccgatctacgtatcagcttcactggtaccaactccattgcgtgaaccga

SEQ ID NO: 497 - DNA, 84 bp, Artificial Sequence
  Probe
aggaccggatcaacttggagttcagacgtgtgctcttccgatcttgattctatggtggggagcgatccgcattgcgtgaaccga

SEQ ID NO: 498 - DNA, 83 bp, Artificial Sequence
  Probe
aggaccggatcaacttggagttcagacgtgtgctcttccgatcttgcggcgagcagcggtagggtgcgcattgcgtgaaccga

SEQ ID NO: 499 - DNA, 85 bp, Artificial Sequence
  Probe
aggaccggatcaacttggagttcagacgtgtgctcttccgatcttagagtgctttctagagctggatgcgcattgcgtgaaccga

SEQ ID NO: 500 - DNA, 86 bp, Artificial Sequence
  Probe
aggaccggatcaacttggagttcagacgtgtgctcttccgatcttgatggatagctctggagatgacatggcattgcgtgaaccga

SEQ ID NO: 501 - DNA, 86 bp, Artificial Sequence
  Probe
aggaccggatcaacttggagttcagacgtgtgctcttccgatcttatgagcgaggtacttaccacgtaatccattgcgtgaaccga

SEQ ID NO: 502 - DNA, 85 bp, Artificial Sequence
  Probe
aggaccggatcaacttggagttcagacgtgtgctcttccgatctacgtacgctgctggatggaaagatggcattgcgtgaaccga

SEQ ID NO: 503 - DNA, 87 bp, Artificial Sequence
  Probe
aggaccggatcaacttggagttcagacgtgtgctcttccgatctgctgtgggaacagtggagtaacaaaatgcattgcgtgaaccga

SEQ ID NO: 504 - DNA, 87 bp, Artificial Sequence
  Probe
aggaccggatcaacttggagttcagacgtgtgctcttccgatctgcgctgtcagaacccagatttactcaagcattgcgtgaaccga

SEQ ID NO: 505 - DNA, 87 bp, Artificial Sequence
  Probe
aggaccggatcaacttggagttcagacgtgtgctcttccgatctcgaccagctgaagtttgtttgaggatagcattgcgtgaaccga

SEQ ID NO: 506 - DNA, 86 bp, Artificial Sequence
  Probe
aggaccggatcaacttggagttcagacgtgtgctcttccgatctacgtagctgctctcttcagtttcagtccattgcgtgaaccga

SEQ ID NO: 507 - DNA, 88 bp, Artificial Sequence
  Probe
aggaccggatcaacttggagttcagacgtgtgctcttccgatctacgtgaaaatcttgcaaaacgttggactccattgcgtgaaccga

SEQ ID NO: 508 - DNA, 85 bp, Artificial Sequence
  Probe
aggaccggatcaacttggagttcagacgtgtgctcttccgatcttgcgcggtatcctttctgtcactgcccattgcgtgaaccga

SEQ ID NO: 509 - DNA, 87 bp, Artificial Sequence
  Probe
aggaccggatcaacttggagttcagacgtgtgctcttccgatcttatgctcatcaagatctttcacagccagcattgcgtgaaccga

SEQ ID NO: 510 - DNA, 85 bp, Artificial Sequence
  Probe
aggaccggatcaacttggagttcagacgtgtgctcttccgatcttgtaattggatgggtaagctgctgggcattgcgtgaaccga

SEQ ID NO: 511 - DNA, 88 bp, Artificial Sequence
  Probe
aggaccggatcaacttggagttcagacgtgtgctcttccgatcttatgtaactttggacgataatcaagagaccattgcgtgaaccga

SEQ ID NO: 512 - DNA, 84 bp, Artificial Sequence
  Probe
aggaccggatcaacttggagttcagacgtgtgctcttccgatcttagaccaatcgagcatcccttgcgccattgcgtgaaccga

SEQ ID NO: 513 - DNA, 86 bp, Artificial Sequence
  Probe
aggaccggatcaacttggagttcagacgtgtgctcttccgatcttgcgggtagcagaggttccacatgaagcattgcgtgaaccga

SEQ ID NO: 514 - DNA, 85 bp, Artificial Sequence
  Probe
aggaccggatcaacttggagttcagacgtgtgctcttccgatctgctgtctaccacatcacaggaccgagcattgcgtgaaccga

SEQ ID NO: 515 - DNA, 86 bp, Artificial Sequence
  Probe
aggaccggatcaacttggagttcagacgtgtgctcttccgatctgtacgttcgtcatggttgacctagatgcattgcgtgaaccga

SEQ ID NO: 516 - DNA, 85 bp, Artificial Sequence
  Probe
aggaccggatcaacttggagttcagacgtgtgctcttccgatctacgtgcaagagacaactccatgagcccattgcgtgaaccga

SEQ ID NO: 517 - DNA, 87 bp, Artificial Sequence
  Probe
aggaccggatcaacttggagttcagacgtgtgctcttccgatctagagaggccggatttcaaaagtttagtccattgcgtgaaccga

SEQ ID NO: 518 - DNA, 86 bp, Artificial Sequence
  Probe
aggaccggatcaacttggagttcagacgtgtgctcttccgatctagagtctcagcacggaaagttctacaccattgcgtgaaccga

SEQ ID NO: 519 - DNA, 88 bp, Artificial Sequence
  Probe
aggaccggatcaacttggagttcagacgtgtgctcttccgatctgtacctctgatttcttccggtttcaatagcattgcgtgaaccga

SEQ ID NO: 520 - DNA, 87 bp, Artificial Sequence
  Probe
aggaccggatcaacttggagttcagacgtgtgctcttccgatctgcgcctgatgtactgatacctttttccgcattgcgtgaaccga

SEQ ID NO: 521 - DNA, 87 bp, Artificial Sequence
  Probe
aggaccggatcaacttggagttcagacgtgtgctcttccgatcttctcttgtgctgaaaacgtgaattctgccattgcgtgaaccga

SEQ ID NO: 522 - DNA, 84 bp, Artificial Sequence
  Probe
aggaccggatcaacttggagttcagacgtgtgctcttccgatctgtctggcccaatcccggcgtctatccattgcgtgaaccga

SEQ ID NO: 523 - DNA, 87 bp, Artificial Sequence
  Probe
aggaccggatcaacttggagttcagacgtgtgctcttccgatcttctcggagtgttgtttccattggtactgcattgcgtgaaccga

SEQ ID NO: 524 - DNA, 85 bp, Artificial Sequence
  Probe
aggaccggatcaacttggagttcagacgtgtgctcttccgatcttatgacctgctggatctgctgaagagcattgcgtgaaccga

SEQ ID NO: 525 - DNA, 88 bp, Artificial Sequence
  Probe
aggaccggatcaacttggagttcagacgtgtgctcttccgatcttgatgttgttacatctcgtttctctttcccattgcgtgaaccga

SEQ ID NO: 526 - DNA, 86 bp, Artificial Sequence
  Probe
aggaccggatcaacttggagttcagacgtgtgctcttccgatcttcagtatccatgtctccaggtgaagtgcattgcgtgaaccga

SEQ ID NO: 527 - DNA, 86 bp, Artificial Sequence
  Probe
aggaccggatcaacttggagttcagacgtgtgctcttccgatctcagcgttcaatgctttacctcctctggcattgcgtgaaccga

SEQ ID NO: 528 - DNA, 83 bp, Artificial Sequence
  Probe
aggaccggatcaacttggagttcagacgtgtgctcttccgatcttctctccagggcatcagcgcctcccattgcgtgaaccga

SEQ ID NO: 529 - DNA, 86 bp, Artificial Sequence
  Probe
aggaccggatcaacttggagttcagacgtgtgctcttccgatctacgtgcaaggtgaagcttcactgaaaccattgcgtgaaccga

SEQ ID NO: 530 - DNA, 83 bp, Artificial Sequence
  Probe
aggaccggatcaacttggagttcagacgtgtgctcttccgatcttgcgacgccagacgacgcgtctcccattgcgtgaaccga

SEQ ID NO: 531 - DNA, 88 bp, Artificial Sequence
  Probe
aggaccggatcaacttggagttcagacgtgtgctcttccgatcttctcaaaacaccaccaccatttcattttgcattgcgtgaaccga

SEQ ID NO: 532 - DNA, 87 bp, Artificial Sequence
  Probe
aggaccggatcaacttggagttcagacgtgtgctcttccgatcttgatatggggaatctctgcatgtaacatcattgcgtgaaccga

SEQ ID NO: 533 - DNA, 86 bp, Artificial Sequence
  Probe
aggaccggatcaacttggagttcagacgtgtgctcttccgatcttctcgcagagccagctaaaagatcaaccattgcgtgaaccga

SEQ ID NO: 534 - DNA, 86 bp, Artificial Sequence
  Probe
aggaccggatcaacttggagttcagacgtgtgctcttccgatctacgtctttagctgcacaactgctatggcattgcgtgaaccga

SEQ ID NO: 535 - DNA, 87 bp, Artificial Sequence
  Probe
aggaccggatcaacttggagttcagacgtgtgctcttccgatcttgcgggtaagctcttgttttgttgctcccattgcgtgaaccga

SEQ ID NO: 536 - DNA, 87 bp, Artificial Sequence
  Probe
aggaccggatcaacttggagttcagacgtgtgctcttccgatctgtctgatgagatgcatacaaaattgccgcattgcgtgaaccga

SEQ ID NO: 537 - DNA, 86 bp, Artificial Sequence
  Probe
aggaccggatcaacttggagttcagacgtgtgctcttccgatcttctcccaggattgttgttctgctttcgcattgcgtgaaccga

SEQ ID NO: 538 - DNA, 86 bp, Artificial Sequence
  Probe
aggaccggatcaacttggagttcagacgtgtgctcttccgatcttcagagcctgattgacaatgttgtccccattgcgtgaaccga

SEQ ID NO: 539 - DNA, 86 bp, Artificial Sequence
  Probe
aggaccggatcaacttggagttcagacgtgtgctcttccgatctcgacgggcactgatctaacaacctgaccattgcgtgaaccga

SEQ ID NO: 540 - DNA, 85 bp, Artificial Sequence
  Probe
aggaccggatcaacttggagttcagacgtgtgctcttccgatctcagccgctgctcgtgtctgaattctccattgcgtgaaccga

SEQ ID NO: 541 - DNA, 86 bp, Artificial Sequence
  Probe
aggaccggatcaacttggagttcagacgtgtgctcttccgatctgcgccacgatgaaggcagcttcttcaccattgcgtgaaccga

SEQ ID NO: 542 - DNA, 88 bp, Artificial Sequence
  Probe
aggaccggatcaacttggagttcagacgtgtgctcttccgatctgctgtctcataatttcaaaatcggatgcgcattgcgtgaaccga

SEQ ID NO: 543 - DNA, 86 bp, Artificial Sequence
  Probe
aggaccggatcaacttggagttcagacgtgtgctcttccgatctgcgcattaaggatgtctatcgaccgggcattgcgtgaaccga

SEQ ID NO: 544 - DNA, 89 bp, Artificial Sequence
  Probe
aggaccggatcaacttggagttcagacgtgtgctcttccgatcttctcagtacaacaagagaaaaagagaaatgcattgcgtgaaccga

SEQ ID NO: 545 - DNA, 87 bp, Artificial Sequence
  Probe
aggaccggatcaacttggagttcagacgtgtgctcttccgatcttagagtatacattgtcttggggcttatccattgcgtgaaccga

SEQ ID NO: 546 - DNA, 87 bp, Artificial Sequence
  Probe
aggaccggatcaacttggagttcagacgtgtgctcttccgatctgtaccctgcatctttgtcctatcctatgcattgcgtgaaccga

SEQ ID NO: 547 - DNA, 86 bp, Artificial Sequence
  Probe
aggaccggatcaacttggagttcagacgtgtgctcttccgatcttagagctgctggaatataattgggggccattgcgtgaaccga

SEQ ID NO: 548 - DNA, 84 bp, Artificial Sequence
  Probe
aggaccggatcaacttggagttcagacgtgtgctcttccgatctgctggaagacccggaccggaaggagcattgcgtgaaccga

SEQ ID NO: 549 - DNA, 90 bp, Artificial Sequence
  Probe
aggaccggatcaacttggagttcagacgtgtgctcttccgatctgctgctctgatactttctttcaaaacataagcattgcgtgaaccga

SEQ ID NO: 550 - DNA, 86 bp, Artificial Sequence
  Probe
aggaccggatcaacttggagttcagacgtgtgctcttccgatctagagcgccataaaagttatgccaccaccattgcgtgaaccga

SEQ ID NO: 551 - DNA, 86 bp, Artificial Sequence
  Probe
aggaccggatcaacttggagttcagacgtgtgctcttccgatcttgcggcgagaaaccacaagttaaacggcattgcgtgaaccga

SEQ ID NO: 552 - DNA, 86 bp, Artificial Sequence
  Probe
aggaccggatcaacttggagttcagacgtgtgctcttccgatcttctcgtcagaaccaatgccgtagtaaccattgcgtgaaccga

SEQ ID NO: 553 - DNA, 86 bp, Artificial Sequence
  Probe
aggaccggatcaacttggagttcagacgtgtgctcttccgatcttatgtctgctgctgttgatagtgctagcattgcgtgaaccga

SEQ ID NO: 554 - DNA, 88 bp, Artificial Sequence
  Probe
aggaccggatcaacttggagttcagacgtgtgctcttccgatcttctcagatcagaccaatgttatcaaactgcattgcgtgaaccga

SEQ ID NO: 555 - DNA, 86 bp, Artificial Sequence
  Probe
aggaccggatcaacttggagttcagacgtgtgctcttccgatctcgacgattaattaatggcccctcctcccattgcgtgaaccga

SEQ ID NO: 556 - DNA, 87 bp, Artificial Sequence
  Probe
aggaccggatcaacttggagttcagacgtgtgctcttccgatcttagaactttgaaccattggatggagatgcattgcgtgaaccga

SEQ ID NO: 557 - DNA, 88 bp, Artificial Sequence
  Probe
aggaccggatcaacttggagttcagacgtgtgctcttccgatcttatgcttaacaccgtaaagtagagataaccattgcgtgaaccga

SEQ ID NO: 558 - DNA, 87 bp, Artificial Sequence
  Probe
aggaccggatcaacttggagttcagacgtgtgctcttccgatcttagacatcaaatgtgaagtcgtcaccaccattgcgtgaaccga

SEQ ID NO: 559 - DNA, 88 bp, Artificial Sequence
  Probe
aggaccggatcaacttggagttcagacgtgtgctcttccgatctcagcctacgagtacatgcatatacagtaccattgcgtgaaccga

SEQ ID NO: 560 - DNA, 86 bp, Artificial Sequence
  Probe
aggaccggatcaacttggagttcagacgtgtgctcttccgatcttcagatattccttgatgggcttctgggcattgcgtgaaccga

SEQ ID NO: 561 - DNA, 84 bp, Artificial Sequence
  Probe
aggaccggatcaacttggagttcagacgtgtgctcttccgatctgcgcgcagccatctctaccgacacccattgcgtgaaccga

SEQ ID NO: 562 - DNA, 89 bp, Artificial Sequence
  Probe
aggaccggatcaacttggagttcagacgtgtgctcttccgatctcagcctttgtttttggccgtgaaataaaatcattgcgtgaaccga

SEQ ID NO: 563 - DNA, 85 bp, Artificial Sequence
  Probe
aggaccggatcaacttggagttcagacgtgtgctcttccgatctcgaccggttagtacgccatagcgaaccattgcgtgaaccga

SEQ ID NO: 564 - DNA, 86 bp, Artificial Sequence
  Probe
aggaccggatcaacttggagttcagacgtgtgctcttccgatcttgcgctgtgctgcgcatttctttgttccattgcgtgaaccga

SEQ ID NO: 565 - DNA, 86 bp, Artificial Sequence
  Probe
aggaccggatcaacttggagttcagacgtgtgctcttccgatcttcagcttctgaaatcgaagtgcgagagcattgcgtgaaccga

SEQ ID NO: 566 - DNA, 85 bp, Artificial Sequence
  Probe
aggaccggatcaacttggagttcagacgtgtgctcttccgatctacgtgccgagccgatcaagatagtggcattgcgtgaaccga

SEQ ID NO: 567 - DNA, 87 bp, Artificial Sequence
  Probe
aggaccggatcaacttggagttcagacgtgtgctcttccgatcttagagtcggtagatcacaagcatgatagcattgcgtgaaccga

SEQ ID NO: 568 - DNA, 86 bp, Artificial Sequence
  Probe
aggaccggatcaacttggagttcagacgtgtgctcttccgatcttgtaagaatgtcttccaaactgcctggcattgcgtgaaccga

SEQ ID NO: 569 - DNA, 88 bp, Artificial Sequence
  Probe
aggaccggatcaacttggagttcagacgtgtgctcttccgatctagagcaaggtttttttgtgaaaggagtggcattgcgtgaaccga

SEQ ID NO: 570 - DNA, 87 bp, Artificial Sequence
  Probe
aggaccggatcaacttggagttcagacgtgtgctcttccgatctgtctttgagggaaatgatctagaatggccattgcgtgaaccga

SEQ ID NO: 571 - DNA, 86 bp, Artificial Sequence
  Probe
aggaccggatcaacttggagttcagacgtgtgctcttccgatctgtacctaatttcagcagcaaactggcccattgcgtgaaccga

SEQ ID NO: 572 - DNA, 85 bp, Artificial Sequence
  Probe
aggaccggatcaacttggagttcagacgtgtgctcttccgatcttctccgtcgtcgttctgacatgcttccattgcgtgaaccga

SEQ ID NO: 573 - DNA, 87 bp, Artificial Sequence
  Probe
aggaccggatcaacttggagttcagacgtgtgctcttccgatcttatgctttagaaatccgggtcatctttccattgcgtgaaccga

SEQ ID NO: 574 - DNA, 86 bp, Artificial Sequence
  Probe
aggaccggatcaacttggagttcagacgtgtgctcttccgatcttgtacgattgcttacactgttgcagcccattgcgtgaaccga

SEQ ID NO: 575 - DNA, 86 bp, Artificial Sequence
  Probe
aggaccggatcaacttggagttcagacgtgtgctcttccgatcttctcagcatatagaagaggggaaggagcattgcgtgaaccga

SEQ ID NO: 576 - DNA, 86 bp, Artificial Sequence
  Probe
aggaccggatcaacttggagttcagacgtgtgctcttccgatctgcgcgagatggttggtgagagtcatagcattgcgtgaaccga

SEQ ID NO: 577 - DNA, 86 bp, Artificial Sequence
  Probe
aggaccggatcaacttggagttcagacgtgtgctcttccgatctcagcgataagcatgtgcagcaacttgccattgcgtgaaccga

SEQ ID NO: 578 - DNA, 85 bp, Artificial Sequence
  Probe
aggaccggatcaacttggagttcagacgtgtgctcttccgatcttagactggacgtagtcgttgtcaacgcattgcgtgaaccga

SEQ ID NO: 579 - DNA, 86 bp, Artificial Sequence
  Probe
aggaccggatcaacttggagttcagacgtgtgctcttccgatctgctgcatagagcgggaaaaaaagtgggcattgcgtgaaccga

SEQ ID NO: 580 - DNA, 89 bp, Artificial Sequence
  Probe
aggaccggatcaacttggagttcagacgtgtgctcttccgatcttgatgttgtaagtgcacaaaaataaagcagcattgcgtgaaccga

SEQ ID NO: 581 - DNA, 87 bp, Artificial Sequence
  Probe
aggaccggatcaacttggagttcagacgtgtgctcttccgatcttgcgccaaattcaagctgcaagttatcccattgcgtgaaccga

SEQ ID NO: 582 - DNA, 87 bp, Artificial Sequence
  Probe
aggaccggatcaacttggagttcagacgtgtgctcttccgatctacgtcacatccgagtgaagagtaaacagcattgcgtgaaccga

SEQ ID NO: 583 - DNA, 86 bp, Artificial Sequence
  Probe
aggaccggatcaacttggagttcagacgtgtgctcttccgatcttatggtaatccacaaagttaccagcgccattgcgtgaaccga

SEQ ID NO: 584 - DNA, 86 bp, Artificial Sequence
  Probe
aggaccggatcaacttggagttcagacgtgtgctcttccgatcttgtatagcatgcctctgttatctgcagcattgcgtgaaccga

SEQ ID NO: 585 - DNA, 85 bp, Artificial Sequence
  Probe
aggaccggatcaacttggagttcagacgtgtgctcttccgatctagagaatgtccaaatcccgccggaaccattgcgtgaaccga

SEQ ID NO: 586 - DNA, 85 bp, Artificial Sequence
  Probe
aggaccggatcaacttggagttcagacgtgtgctcttccgatcttagagctggtagcagccatgcatctgcattgcgtgaaccga

SEQ ID NO: 587 - DNA, 86 bp, Artificial Sequence
  Probe
aggaccggatcaacttggagttcagacgtgtgctcttccgatctagagggtatgaccaaactaagtcgacgcattgcgtgaaccga

SEQ ID NO: 588 - DNA, 87 bp, Artificial Sequence
  Probe
aggaccggatcaacttggagttcagacgtgtgctcttccgatcttgtagaaagcaccacaatcaggtcaaaccattgcgtgaaccga

SEQ ID NO: 589 - DNA, 88 bp, Artificial Sequence
  Probe
aggaccggatcaacttggagttcagacgtgtgctcttccgatcttagatgtgaactgaagtagtttctttgtccattgcgtgaaccga

SEQ ID NO: 590 - DNA, 87 bp, Artificial Sequence
  Probe
aggaccggatcaacttggagttcagacgtgtgctcttccgatctgtcttgaaaatgaggcagcactttcatccattgcgtgaaccga

SEQ ID NO: 591 - DNA, 86 bp, Artificial Sequence
  Probe
aggaccggatcaacttggagttcagacgtgtgctcttccgatcttatgtcgtaaaagctatggctgcagagcattgcgtgaaccga

SEQ ID NO: 592 - DNA, 86 bp, Artificial Sequence
  Probe
aggaccggatcaacttggagttcagacgtgtgctcttccgatctgctgtatggacggtgctcacaaaatggcattgcgtgaaccga

SEQ ID NO: 593 - DNA, 85 bp, Artificial Sequence
  Probe
aggaccggatcaacttggagttcagacgtgtgctcttccgatctgtctgccggcaagctgagtaatttggcattgcgtgaaccga

SEQ ID NO: 594 - DNA, 87 bp, Artificial Sequence
  Probe
aggaccggatcaacttggagttcagacgtgtgctcttccgatcttatgcagtacagtctcaagcaatcgatccattgcgtgaaccga

SEQ ID NO: 595 - DNA, 87 bp, Artificial Sequence
  Probe
aggaccggatcaacttggagttcagacgtgtgctcttccgatcttgtacttaaacatcctagatcggctctgcattgcgtgaaccga

SEQ ID NO: 596 - DNA, 86 bp, Artificial Sequence
  Probe
aggaccggatcaacttggagttcagacgtgtgctcttccgatctagaggttagttgtcttgcgctcatgcccattgcgtgaaccga

SEQ ID NO: 597 - DNA, 85 bp, Artificial Sequence
  Probe
aggaccggatcaacttggagttcagacgtgtgctcttccgatctacgtgtctaggcctcctaagcttacccattgcgtgaaccga

SEQ ID NO: 598 - DNA, 88 bp, Artificial Sequence
  Probe
aggaccggatcaacttggagttcagacgtgtgctcttccgatcttgatagcaagctctattacatcaaagaaccattgcgtgaaccga

SEQ ID NO: 599 - DNA, 85 bp, Artificial Sequence
  Probe
aggaccggatcaacttggagttcagacgtgtgctcttccgatcttgcggacagcatgcagcatcgttgcgcattgcgtgaaccga

SEQ ID NO: 600 - DNA, 85 bp, Artificial Sequence
  Probe
aggaccggatcaacttggagttcagacgtgtgctcttccgatctcagccacccccttagatgctctatgccattgcgtgaaccga

SEQ ID NO: 601 - DNA, 86 bp, Artificial Sequence
  Probe
aggaccggatcaacttggagttcagacgtgtgctcttccgatcttgtactgtagagggcagcaagtttcatcattgcgtgaaccga

SEQ ID NO: 602 - DNA, 88 bp, Artificial Sequence
  Probe
aggaccggatcaacttggagttcagacgtgtgctcttccgatctcagcgacaaaagaaaaaggacacatgaagcattgcgtgaaccga

SEQ ID NO: 603 - DNA, 88 bp, Artificial Sequence
  Probe
aggaccggatcaacttggagttcagacgtgtgctcttccgatcttctccgtattagtacagtatttcagagtgcattgcgtgaaccga

SEQ ID NO: 604 - DNA, 84 bp, Artificial Sequence
  Probe
aggaccggatcaacttggagttcagacgtgtgctcttccgatcttgtagcttgggctgcatcgcctgagcattgcgtgaaccga

SEQ ID NO: 605 - DNA, 88 bp, Artificial Sequence
  Probe
aggaccggatcaacttggagttcagacgtgtgctcttccgatctgtacgattttcagctttgcactaactgaccattgcgtgaaccga

SEQ ID NO: 606 - DNA, 89 bp, Artificial Sequence
  Probe
aggaccggatcaacttggagttcagacgtgtgctcttccgatctcagccaaagttgatatcttttccaatcttccattgcgtgaaccga

SEQ ID NO: 607 - DNA, 87 bp, Artificial Sequence
  Probe
aggaccggatcaacttggagttcagacgtgtgctcttccgatcttctccctgatgaaggcaaaagggaaaagcattgcgtgaaccga

SEQ ID NO: 608 - DNA, 86 bp, Artificial Sequence
  Probe
aggaccggatcaacttggagttcagacgtgtgctcttccgatctacgtgcaaacccggatcagtaacaatccattgcgtgaaccga

SEQ ID NO: 609 - DNA, 88 bp, Artificial Sequence
  Probe
aggaccggatcaacttggagttcagacgtgtgctcttccgatctcgactgattgcagttggtttcattttgaccattgcgtgaaccga

SEQ ID NO: 610 - DNA, 85 bp, Artificial Sequence
  Probe
aggaccggatcaacttggagttcagacgtgtgctcttccgatctgctgcgcaatacagcggtcacaacaccattgcgtgaaccga

SEQ ID NO: 611 - DNA, 90 bp, Artificial Sequence
  Probe
aggaccggatcaacttggagttcagacgtgtgctcttccgatctgcgccaataagattagcataaaatagtcgtgcattgcgtgaaccga

SEQ ID NO: 612 - DNA, 88 bp, Artificial Sequence
  Probe
aggaccggatcaacttggagttcagacgtgtgctcttccgatcttgtattttcaccaaaattaagcaggactgcattgcgtgaaccga

SEQ ID NO: 613 - DNA, 85 bp, Artificial Sequence
  Probe
aggaccggatcaacttggagttcagacgtgtgctcttccgatctgtacggtggttattcgggcttttgcgcattgcgtgaaccga

SEQ ID NO: 614 - DNA, 86 bp, Artificial Sequence
  Probe
aggaccggatcaacttggagttcagacgtgtgctcttccgatcttgcgagtggcattcagatcaacagtcccattgcgtgaaccga

SEQ ID NO: 615 - DNA, 86 bp, Artificial Sequence
  Probe
aggaccggatcaacttggagttcagacgtgtgctcttccgatctgcgcgagagagagagagagagagatcgcattgcgtgaaccga

SEQ ID NO: 616 - DNA, 86 bp, Artificial Sequence
  Probe
aggaccggatcaacttggagttcagacgtgtgctcttccgatcttcaggccagtaactctttcctccctaccattgcgtgaaccga

SEQ ID NO: 617 - DNA, 86 bp, Artificial Sequence
  Probe
aggaccggatcaacttggagttcagacgtgtgctcttccgatctgctgcaaaggagctagatcttcttcggcattgcgtgaaccga

SEQ ID NO: 618 - DNA, 88 bp, Artificial Sequence
  Probe
aggaccggatcaacttggagttcagacgtgtgctcttccgatctgcgcgttgaactctttgaacacatcattgcattgcgtgaaccga

SEQ ID NO: 619 - DNA, 87 bp, Artificial Sequence
  Probe
aggaccggatcaacttggagttcagacgtgtgctcttccgatcttctcgaagaacacaaggcagattgatgccattgcgtgaaccga

SEQ ID NO: 620 - DNA, 87 bp, Artificial Sequence
  Probe
aggaccggatcaacttggagttcagacgtgtgctcttccgatcttcaggcaagtttgtatacttcaggggtgcattgcgtgaaccga

SEQ ID NO: 621 - DNA, 84 bp, Artificial Sequence
  Probe
aggaccggatcaacttggagttcagacgtgtgctcttccgatctacgtgacgtccggctgctactactccattgcgtgaaccga

SEQ ID NO: 622 - DNA, 88 bp, Artificial Sequence
  Probe
aggaccggatcaacttggagttcagacgtgtgctcttccgatctgtctgactgtagttttgtgcatcttgaaccattgcgtgaaccga

SEQ ID NO: 623 - DNA, 90 bp, Artificial Sequence
  Probe
aggaccggatcaacttggagttcagacgtgtgctcttccgatcttgatcagttgagttcgtttatttatttatagcattgcgtgaaccga

SEQ ID NO: 624 - DNA, 85 bp, Artificial Sequence
  Probe
aggaccggatcaacttggagttcagacgtgtgctcttccgatcttatgaattggtagggaaggggttccgcattgcgtgaaccga

SEQ ID NO: 625 - DNA, 85 bp, Artificial Sequence
  Probe
aggaccggatcaacttggagttcagacgtgtgctcttccgatctgtctccagcaccatgaaggttcatcccattgcgtgaaccga

SEQ ID NO: 626 - DNA, 86 bp, Artificial Sequence
  Probe
aggaccggatcaacttggagttcagacgtgtgctcttccgatcttagagaagcatggccggttatatactccattgcgtgaaccga

SEQ ID NO: 627 - DNA, 86 bp, Artificial Sequence
  Probe
aggaccggatcaacttggagttcagacgtgtgctcttccgatcttatgtccacagtaatgtaaccactgcccattgcgtgaaccga

SEQ ID NO: 628 - DNA, 87 bp, Artificial Sequence
  Probe
aggaccggatcaacttggagttcagacgtgtgctcttccgatctgtctcttcttgtcaaaaatgaggccaggcattgcgtgaaccga

SEQ ID NO: 629 - DNA, 88 bp, Artificial Sequence
  Probe
aggaccggatcaacttggagttcagacgtgtgctcttccgatcttagacgaaaataaccaaactgcacttctgcattgcgtgaaccga

SEQ ID NO: 630 - DNA, 89 bp, Artificial Sequence
  Probe
aggaccggatcaacttggagttcagacgtgtgctcttccgatcttagaagaaaaatttaggcagcacaaaaatgcattgcgtgaaccga

SEQ ID NO: 631 - DNA, 87 bp, Artificial Sequence
  Probe
aggaccggatcaacttggagttcagacgtgtgctcttccgatcttatggttggaaaatcggtgtaccatatgcattgcgtgaaccga

SEQ ID NO: 632 - DNA, 90 bp, Artificial Sequence
  Probe
aggaccggatcaacttggagttcagacgtgtgctcttccgatcttgcgggtttggttcgttatattatatatagccattgcgtgaaccga

SEQ ID NO: 633 - DNA, 84 bp, Artificial Sequence
  Probe
aggaccggatcaacttggagttcagacgtgtgctcttccgatcttagaggcagccatgtcagctacagccattgcgtgaaccga

SEQ ID NO: 634 - DNA, 85 bp, Artificial Sequence
  Probe
aggaccggatcaacttggagttcagacgtgtgctcttccgatcttgcgccagctctacaccaaggaatcgcattgcgtgaaccga

SEQ ID NO: 635 - DNA, 87 bp, Artificial Sequence
  Probe
aggaccggatcaacttggagttcagacgtgtgctcttccgatctgcgcaacctttgaagagaacgtgcataccattgcgtgaaccga

SEQ ID NO: 636 - DNA, 87 bp, Artificial Sequence
  Probe
aggaccggatcaacttggagttcagacgtgtgctcttccgatcttagaggcaaggattatctaagctgctaccattgcgtgaaccga

SEQ ID NO: 637 - DNA, 85 bp, Artificial Sequence
  Probe
aggaccggatcaacttggagttcagacgtgtgctcttccgatctgcgcgaccagactaccagagacagaccattgcgtgaaccga

SEQ ID NO: 638 - DNA, 87 bp, Artificial Sequence
  Probe
aggaccggatcaacttggagttcagacgtgtgctcttccgatcttgtatgagttctgtttattttggctgcgcattgcgtgaaccga

SEQ ID NO: 639 - DNA, 85 bp, Artificial Sequence
  Probe
aggaccggatcaacttggagttcagacgtgtgctcttccgatctagagcgactacgatgcccccattgaccattgcgtgaaccga

SEQ ID NO: 640 - DNA, 88 bp, Artificial Sequence
  Probe
aggaccggatcaacttggagttcagacgtgtgctcttccgatctgtaccatgaaacgacaacacattcacatccattgcgtgaaccga

SEQ ID NO: 641 - DNA, 87 bp, Artificial Sequence
  Probe
aggaccggatcaacttggagttcagacgtgtgctcttccgatcttagagcaattgtgtttggaggcatacagcattgcgtgaaccga

SEQ ID NO: 642 - DNA, 89 bp, Artificial Sequence
  Probe
aggaccggatcaacttggagttcagacgtgtgctcttccgatctgctggaatgaagatgtgattatgctattaccattgcgtgaaccga

SEQ ID NO: 643 - DNA, 87 bp, Artificial Sequence
  Probe
aggaccggatcaacttggagttcagacgtgtgctcttccgatcttgtagccatttttcacatccagtgatcgcattgcgtgaaccga

SEQ ID NO: 644 - DNA, 86 bp, Artificial Sequence
  Probe
aggaccggatcaacttggagttcagacgtgtgctcttccgatctgtctgcgtaatgagtccttgcagtacccattgcgtgaaccga

SEQ ID NO: 645 - DNA, 87 bp, Artificial Sequence
  Probe
aggaccggatcaacttggagttcagacgtgtgctcttccgatctacgtacaaatgggttatgcagaagtagccattgcgtgaaccga

SEQ ID NO: 646 - DNA, 88 bp, Artificial Sequence
  Probe
aggaccggatcaacttggagttcagacgtgtgctcttccgatcttagatatatacgcatttgatgtgcatgtccattgcgtgaaccga

SEQ ID NO: 647 - DNA, 83 bp, Artificial Sequence
  Probe
aggaccggatcaacttggagttcagacgtgtgctcttccgatcttgatccgggcttcccaccaaacgccattgcgtgaaccga

SEQ ID NO: 648 - DNA, 86 bp, Artificial Sequence
  Probe
aggaccggatcaacttggagttcagacgtgtgctcttccgatctgcgcttttaggaaggccagagtacacgcattgcgtgaaccga

SEQ ID NO: 649 - DNA, 87 bp, Artificial Sequence
  Probe
aggaccggatcaacttggagttcagacgtgtgctcttccgatcttgtacattgtttccacatcctccttaggcattgcgtgaaccga

SEQ ID NO: 650 - DNA, 87 bp, Artificial Sequence
  Probe
aggaccggatcaacttggagttcagacgtgtgctcttccgatcttgcgcccacacactctcttgtcaatatccattgcgtgaaccga

SEQ ID NO: 651 - DNA, 86 bp, Artificial Sequence
  Probe
aggaccggatcaacttggagttcagacgtgtgctcttccgatctagagcaggttcttggatgtttatggcccattgcgtgaaccga

SEQ ID NO: 652 - DNA, 85 bp, Artificial Sequence
  Probe
aggaccggatcaacttggagttcagacgtgtgctcttccgatctgtctagcaccgtgtccctgtatgtagcattgcgtgaaccga

SEQ ID NO: 653 - DNA, 90 bp, Artificial Sequence
  Probe
aggaccggatcaactcgacaggagcaggctgtcctgagctctgaagatcggaagagcgtcgtgtagggaaagagtcattgcgtgaaccga

SEQ ID NO: 654 - DNA, 92 bp, Artificial Sequence
  Probe
aggaccggatcaactaactggggtctcaagaaagtccatcgcacacagatcggaagagcgtcgtgtagggaaagagtcattgcgtgaaccga

SEQ ID NO: 655 - DNA, 92 bp, Artificial Sequence
  Probe
aggaccggatcaactggagtcatggaagttggagacattactctacagatcggaagagcgtcgtgtagggaaagagtcattgcgtgaaccga

SEQ ID NO: 656 - DNA, 93 bp, Artificial Sequence
  Probe
aggaccggatcaacttcatctacgatgcacatcaataccgtagagtcagatcggaagagcgtcgtgtagggaaagagtcattgcgtgaaccga

SEQ ID NO: 657 - DNA, 93 bp, Artificial Sequence
  Probe
aggaccggatcaactatttgaacttccctccaaaagtcctagactacagatcggaagagcgtcgtgtagggaaagagtcattgcgtgaaccga

SEQ ID NO: 658 - DNA, 93 bp, Artificial Sequence
  Probe
aggaccggatcaacttaccttgcaaccggtatatgatccgtcgactaagatcggaagagcgtcgtgtagggaaagagtcattgcgtgaaccga

SEQ ID NO: 659 - DNA, 93 bp, Artificial Sequence
  Probe
aggaccggatcaactcaagttcaaaagcagcaaaaggtggctagcagagatcggaagagcgtcgtgtagggaaagagtcattgcgtgaaccga

SEQ ID NO: 660 - DNA, 92 bp, Artificial Sequence
  Probe
aggaccggatcaactatgagctgcaactggaagttcagacagactgagatcggaagagcgtcgtgtagggaaagagtcattgcgtgaaccga

SEQ ID NO: 661 - DNA, 92 bp, Artificial Sequence
  Probe
aggaccggatcaactgcactgtagctgcagacttaacacgtagcgcagatcggaagagcgtcgtgtagggaaagagtcattgcgtgaaccga

SEQ ID NO: 662 - DNA, 91 bp, Artificial Sequence
  Probe
aggaccggatcaactagttcagctgggtggcacagagtagtgataagatcggaagagcgtcgtgtagggaaagagtcattgcgtgaaccga

SEQ ID NO: 663 - DNA, 91 bp, Artificial Sequence
  Probe
aggaccggatcaactctcccgatcccgaccaactaacgtacgatcagatcggaagagcgtcgtgtagggaaagagtcattgcgtgaaccga

SEQ ID NO: 664 - DNA, 91 bp, Artificial Sequence
  Probe
aggaccggatcaactgttcttggcacctgcaagagaccgacatcaagatcggaagagcgtcgtgtagggaaagagtcattgcgtgaaccga

SEQ ID NO: 665 - DNA, 89 bp, Artificial Sequence
  Probe
aggaccggatcaactgtcccatacccgcccgttgcgctactatagatcggaagagcgtcgtgtagggaaagagtcattgcgtgaaccga

SEQ ID NO: 666 - DNA, 92 bp, Artificial Sequence
  Probe
aggaccggatcaactctctaaaaagtcgtacctgagcgagtcatatagatcggaagagcgtcgtgtagggaaagagtcattgcgtgaaccga

SEQ ID NO: 667 - DNA, 91 bp, Artificial Sequence
  Probe
aggaccggatcaactgtaaacgcgctatagggagggtagtgtagaagatcggaagagcgtcgtgtagggaaagagtcattgcgtgaaccga

SEQ ID NO: 668 - DNA, 92 bp, Artificial Sequence
  Probe
aggaccggatcaactagagagagagttcatgccagtggcgacgcacagatcggaagagcgtcgtgtagggaaagagtcattgcgtgaaccga

SEQ ID NO: 669 - DNA, 93 bp, Artificial Sequence
  Probe
aggaccggatcaactatgtccaagtgaagtgatcttggtagagtgcgagatcggaagagcgtcgtgtagggaaagagtcattgcgtgaaccga

SEQ ID NO: 670 - DNA, 93 bp, Artificial Sequence
  Probe
aggaccggatcaacttctgaagatattggagctcagcttactcagctagatcggaagagcgtcgtgtagggaaagagtcattgcgtgaaccga

SEQ ID NO: 671 - DNA, 91 bp, Artificial Sequence
  Probe
aggaccggatcaacttgacgcgcttggtacaacatcctgctagtgagatcggaagagcgtcgtgtagggaaagagtcattgcgtgaaccga

SEQ ID NO: 672 - DNA, 92 bp, Artificial Sequence
  Probe
aggaccggatcaactcggtccttgttgtgaaggttgtagtgcatcgagatcggaagagcgtcgtgtagggaaagagtcattgcgtgaaccga

SEQ ID NO: 673 - DNA, 93 bp, Artificial Sequence
  Probe
aggaccggatcaactattaaggtgttgatccgttgtagcgtgtgtctagatcggaagagcgtcgtgtagggaaagagtcattgcgtgaaccga

SEQ ID NO: 674 - DNA, 93 bp, Artificial Sequence
  Probe
aggaccggatcaactgatcctaataattcccacgcatgtagctgtcgagatcggaagagcgtcgtgtagggaaagagtcattgcgtgaaccga

SEQ ID NO: 675 - DNA, 91 bp, Artificial Sequence
  Probe
aggaccggatcaacttatggatgctgcgttgccaccctgagcataagatcggaagagcgtcgtgtagggaaagagtcattgcgtgaaccga

SEQ ID NO: 676 - DNA, 93 bp, Artificial Sequence
  Probe
aggaccggatcaactgaggcaccacttaaatggttttctactactgcagatcggaagagcgtcgtgtagggaaagagtcattgcgtgaaccga

SEQ ID NO: 677 - DNA, 92 bp, Artificial Sequence
  Probe
aggaccggatcaactgcacaatcagacacagcaataggtagagtatagatcggaagagcgtcgtgtagggaaagagtcattgcgtgaaccga

SEQ ID NO: 678 - DNA, 92 bp, Artificial Sequence
  Probe
aggaccggatcaacttgcatttcttggctgcaagtctgagagcatgagatcggaagagcgtcgtgtagggaaagagtcattgcgtgaaccga

SEQ ID NO: 679 - DNA, 93 bp, Artificial Sequence
  Probe
aggaccggatcaactcacaagatggaatggaagagctagcagatagtagatcggaagagcgtcgtgtagggaaagagtcattgcgtgaaccga

SEQ ID NO: 680 - DNA, 92 bp, Artificial Sequence
  Probe
aggaccggatcaactggagtggacagaatgaaactgaccatgtgacagatcggaagagcgtcgtgtagggaaagagtcattgcgtgaaccga

SEQ ID NO: 681 - DNA, 91 bp, Artificial Sequence
  Probe
aggaccggatcaactgcctctctggatagcacacaagctcgctgtagatcggaagagcgtcgtgtagggaaagagtcattgcgtgaaccga

SEQ ID NO: 682 - DNA, 91 bp, Artificial Sequence
  Probe
aggaccggatcaactgaggcctcacgcacaacaacatctgactcgagatcggaagagcgtcgtgtagggaaagagtcattgcgtgaaccga

SEQ ID NO: 683 - DNA, 92 bp, Artificial Sequence
  Probe
aggaccggatcaactctgactttctgccggggtaaaaacgatactgagatcggaagagcgtcgtgtagggaaagagtcattgcgtgaaccga

SEQ ID NO: 684 - DNA, 92 bp, Artificial Sequence
  Probe
aggaccggatcaactcaatacagatacggacgaccgatgcatctgaagatcggaagagcgtcgtgtagggaaagagtcattgcgtgaaccga

SEQ ID NO: 685 - DNA, 92 bp, Artificial Sequence
  Probe
aggaccggatcaacttactactcaacaaagctcgccgctgtgtcacagatcggaagagcgtcgtgtagggaaagagtcattgcgtgaaccga

SEQ ID NO: 686 - DNA, 93 bp, Artificial Sequence
  Probe
aggaccggatcaactgaggtaatgtatgtttccagtgacactatactagatcggaagagcgtcgtgtagggaaagagtcattgcgtgaaccga

SEQ ID NO: 687 - DNA, 93 bp, Artificial Sequence
  Probe
aggaccggatcaactaaccaataattacgcgtgaacgtcctgatcgaagatcggaagagcgtcgtgtagggaaagagtcattgcgtgaaccga

SEQ ID NO: 688 - DNA, 89 bp, Artificial Sequence
  Probe
aggaccggatcaactggaaccagcggccaggatcgagcacatgagatcggaagagcgtcgtgtagggaaagagtcattgcgtgaaccga

SEQ ID NO: 689 - DNA, 94 bp, Artificial Sequence
  Probe
aggaccggatcaactggtcttcagtaaaatcactcatgtaacgtatagagatcggaagagcgtcgtgtagggaaagagtcattgcgtgaaccga

SEQ ID NO: 690 - DNA, 93 bp, Artificial Sequence
  Probe
aggaccggatcaactgaatggaattagatcatccggatgtacagacgagatcggaagagcgtcgtgtagggaaagagtcattgcgtgaaccga

SEQ ID NO: 691 - DNA, 91 bp, Artificial Sequence
  Probe
aggaccggatcaactcgtgactggaacatcggacagcctgatgacagatcggaagagcgtcgtgtagggaaagagtcattgcgtgaaccga

SEQ ID NO: 692 - DNA, 95 bp, Artificial Sequence
  Probe
aggaccggatcaactttttgaaatttgctgctgataagttgatgctataagatcggaagagcgtcgtgtagggaaagagtcattgcgtgaaccga

SEQ ID NO: 693 - DNA, 92 bp, Artificial Sequence
  Probe
aggaccggatcaactcaactactatcgtacacagctgcactctcacagatcggaagagcgtcgtgtagggaaagagtcattgcgtgaaccga

SEQ ID NO: 694 - DNA, 93 bp, Artificial Sequence
  Probe
aggaccggatcaactggcacttactagttactacgtacctgtgatcgagatcggaagagcgtcgtgtagggaaagagtcattgcgtgaaccga

SEQ ID NO: 695 - DNA, 93 bp, Artificial Sequence
  Probe
aggaccggatcaactcgagttgctgcagatattggtaagctcgtcgaagatcggaagagcgtcgtgtagggaaagagtcattgcgtgaaccga

SEQ ID NO: 696 - DNA, 94 bp, Artificial Sequence
  Probe
aggaccggatcaactagatagatgggcacaaaatggattccgacgctaagatcggaagagcgtcgtgtagggaaagagtcattgcgtgaaccga

SEQ ID NO: 697 - DNA, 93 bp, Artificial Sequence
  Probe
aggaccggatcaactacctctgaaagtttttgtgctgctatcgtagaagatcggaagagcgtcgtgtagggaaagagtcattgcgtgaaccga

SEQ ID NO: 698 - DNA, 92 bp, Artificial Sequence
  Probe
aggaccggatcaactgcaagcacctgacattgatgctcatcagctgagatcggaagagcgtcgtgtagggaaagagtcattgcgtgaaccga

SEQ ID NO: 699 - DNA, 93 bp, Artificial Sequence
  Probe
aggaccggatcaactcagtcagcgtaacaatgctttgatgtagacgcagatcggaagagcgtcgtgtagggaaagagtcattgcgtgaaccga

SEQ ID NO: 700 - DNA, 92 bp, Artificial Sequence
  Probe
aggaccggatcaactccgtacatctttcagcatgacccgcagcgacagatcggaagagcgtcgtgtagggaaagagtcattgcgtgaaccga

SEQ ID NO: 701 - DNA, 92 bp, Artificial Sequence
  Probe
aggaccggatcaactgtgcaaccgagcctatatatgcaagatactaagatcggaagagcgtcgtgtagggaaagagtcattgcgtgaaccga

SEQ ID NO: 702 - DNA, 93 bp, Artificial Sequence
  Probe
aggaccggatcaactaatccccaaccacatttatgtagcctgacagtagatcggaagagcgtcgtgtagggaaagagtcattgcgtgaaccga

SEQ ID NO: 703 - DNA, 92 bp, Artificial Sequence
  Probe
aggaccggatcaactgctcacaagctgaaacaggaacagcgctgatagatcggaagagcgtcgtgtagggaaagagtcattgcgtgaaccga

SEQ ID NO: 704 - DNA, 92 bp, Artificial Sequence
  Probe
aggaccggatcaactaagctccatccaacctgatctgctcgcactaagatcggaagagcgtcgtgtagggaaagagtcattgcgtgaaccga

SEQ ID NO: 705 - DNA, 91 bp, Artificial Sequence
  Probe
aggaccggatcaactgctcgggagcctgctaaagataactcatacagatcggaagagcgtcgtgtagggaaagagtcattgcgtgaaccga

SEQ ID NO: 706 - DNA, 94 bp, Artificial Sequence
  Probe
aggaccggatcaacttcttgttcagtgccatagaaaaaagagcagctcagatcggaagagcgtcgtgtagggaaagagtcattgcgtgaaccga

SEQ ID NO: 707 - DNA, 92 bp, Artificial Sequence
  Probe
aggaccggatcaacttcgatgaagatcctggaaccgacctgtcactagatcggaagagcgtcgtgtagggaaagagtcattgcgtgaaccga

SEQ ID NO: 708 - DNA, 95 bp, Artificial Sequence
  Probe
aggaccggatcaactcttcaatttttcacaaatagtgcatgcatcgtgtagatcggaagagcgtcgtgtagggaaagagtcattgcgtgaaccga

SEQ ID NO: 709 - DNA, 90 bp, Artificial Sequence
  Probe
aggaccggatcaactacctgcaagacaggcgcaccctcgacgcgagatcggaagagcgtcgtgtagggaaagagtcattgcgtgaaccga

SEQ ID NO: 710 - DNA, 92 bp, Artificial Sequence
  Probe
aggaccggatcaactggtagctcgtgaaagctaagcttatacgtacagatcggaagagcgtcgtgtagggaaagagtcattgcgtgaaccga

SEQ ID NO: 711 - DNA, 92 bp, Artificial Sequence
  Probe
aggaccggatcaacttgtgtattcgcactccacctgacgcatgcatagatcggaagagcgtcgtgtagggaaagagtcattgcgtgaaccga

SEQ ID NO: 712 - DNA, 91 bp, Artificial Sequence
  Probe
aggaccggatcaactaatccggtggtactgtacacggcacgagacagatcggaagagcgtcgtgtagggaaagagtcattgcgtgaaccga

SEQ ID NO: 713 - DNA, 91 bp, Artificial Sequence
  Probe
aggaccggatcaactcagcagagaggttgttggatccgagtatctagatcggaagagcgtcgtgtagggaaagagtcattgcgtgaaccga

SEQ ID NO: 714 - DNA, 93 bp, Artificial Sequence
  Probe
aggaccggatcaacttcacagaaagagagcattacggtttgctgataagatcggaagagcgtcgtgtagggaaagagtcattgcgtgaaccga

SEQ ID NO: 715 - DNA, 91 bp, Artificial Sequence
  Probe
aggaccggatcaactatccgccattgtaggccatgacagtagcgaagatcggaagagcgtcgtgtagggaaagagtcattgcgtgaaccga

SEQ ID NO: 716 - DNA, 92 bp, Artificial Sequence
  Probe
aggaccggatcaactgttcaattcgcaagctggagtagctagatgaagatcggaagagcgtcgtgtagggaaagagtcattgcgtgaaccga

SEQ ID NO: 717 - DNA, 89 bp, Artificial Sequence
  Probe
aggaccggatcaactggaggcaatggtggtgggggtagactcgagatcggaagagcgtcgtgtagggaaagagtcattgcgtgaaccga

SEQ ID NO: 718 - DNA, 90 bp, Artificial Sequence
  Probe
aggaccggatcaactgtccagggatcgtcttccccagtagtgtgagatcggaagagcgtcgtgtagggaaagagtcattgcgtgaaccga

SEQ ID NO: 719 - DNA, 92 bp, Artificial Sequence
  Probe
aggaccggatcaactagagtttgccatctgctgcatgcgagatgagagatcggaagagcgtcgtgtagggaaagagtcattgcgtgaaccga

SEQ ID NO: 720 - DNA, 91 bp, Artificial Sequence
  Probe
aggaccggatcaacttccatcgacagagcttgcgagcctatagctagatcggaagagcgtcgtgtagggaaagagtcattgcgtgaaccga

SEQ ID NO: 721 - DNA, 92 bp, Artificial Sequence
  Probe
aggaccggatcaactagtcctagtgcttgtcctcaatcatgctgagagatcggaagagcgtcgtgtagggaaagagtcattgcgtgaaccga

SEQ ID NO: 722 - DNA, 93 bp, Artificial Sequence
  Probe
aggaccggatcaactgtctccttgaagagctgttcaaagcgctacgtagatcggaagagcgtcgtgtagggaaagagtcattgcgtgaaccga

SEQ ID NO: 723 - DNA, 90 bp, Artificial Sequence
  Probe
aggaccggatcaactcagagagaggtcgtggttggggcgcatacagatcggaagagcgtcgtgtagggaaagagtcattgcgtgaaccga

SEQ ID NO: 724 - DNA, 92 bp, Artificial Sequence
  Probe
aggaccggatcaacttctacaatgacccgtggcaagttgtcacgctagatcggaagagcgtcgtgtagggaaagagtcattgcgtgaaccga

SEQ ID NO: 725 - DNA, 92 bp, Artificial Sequence
  Probe
aggaccggatcaacttcgttcctttctttccatcgtcggacatacaagatcggaagagcgtcgtgtagggaaagagtcattgcgtgaaccga

SEQ ID NO: 726 - DNA, 93 bp, Artificial Sequence
  Probe
aggaccggatcaactagcttcatgtgcactccaaactatgcactagaagatcggaagagcgtcgtgtagggaaagagtcattgcgtgaaccga

SEQ ID NO: 727 - DNA, 94 bp, Artificial Sequence
  Probe
aggaccggatcaactacacacatttgatgaagcaacgaatcagtctgaagatcggaagagcgtcgtgtagggaaagagtcattgcgtgaaccga

SEQ ID NO: 728 - DNA, 91 bp, Artificial Sequence
  Probe
aggaccggatcaactccttcagtctctgccagtctgcatacacgcagatcggaagagcgtcgtgtagggaaagagtcattgcgtgaaccga

SEQ ID NO: 729 - DNA, 93 bp, Artificial Sequence
  Probe
aggaccggatcaactacatgaaggtcaacaccaagatcaagctatgtagatcggaagagcgtcgtgtagggaaagagtcattgcgtgaaccga

SEQ ID NO: 730 - DNA, 93 bp, Artificial Sequence
  Probe
aggaccggatcaactagaccaattcagatgccacacttttgcacgtcagatcggaagagcgtcgtgtagggaaagagtcattgcgtgaaccga

SEQ ID NO: 731 - DNA, 90 bp, Artificial Sequence
  Probe
aggaccggatcaactctgtcgcgctccaggtactccgtctagtaagatcggaagagcgtcgtgtagggaaagagtcattgcgtgaaccga

SEQ ID NO: 732 - DNA, 91 bp, Artificial Sequence
  Probe
aggaccggatcaactgaagacatggtaccggagcttcagcgagacagatcggaagagcgtcgtgtagggaaagagtcattgcgtgaaccga

SEQ ID NO: 733 - DNA, 91 bp, Artificial Sequence
  Probe
aggaccggatcaactagctagcatggcatctcgacgaagctcagtagatcggaagagcgtcgtgtagggaaagagtcattgcgtgaaccga

SEQ ID NO: 734 - DNA, 93 bp, Artificial Sequence
  Probe
aggaccggatcaacttgttgccaaaattcgcacgttagtcgatatagagatcggaagagcgtcgtgtagggaaagagtcattgcgtgaaccga

SEQ ID NO: 735 - DNA, 94 bp, Artificial Sequence
  Probe
aggaccggatcaactttgcttgtttattggaacagccattgatacgatagatcggaagagcgtcgtgtagggaaagagtcattgcgtgaaccga

SEQ ID NO: 736 - DNA, 93 bp, Artificial Sequence
  Probe
aggaccggatcaacttttacttcacctgctctctctctgcgacatatagatcggaagagcgtcgtgtagggaaagagtcattgcgtgaaccga

SEQ ID NO: 737 - DNA, 91 bp, Artificial Sequence
  Probe
aggaccggatcaacttcgacggtgacatgccacttccatctagagagatcggaagagcgtcgtgtagggaaagagtcattgcgtgaaccga

SEQ ID NO: 738 - DNA, 93 bp, Artificial Sequence
  Probe
aggaccggatcaactttgcagcaaattgttcgttgcatctgcgtgtaagatcggaagagcgtcgtgtagggaaagagtcattgcgtgaaccga

SEQ ID NO: 739 - DNA, 93 bp, Artificial Sequence
  Probe
aggaccggatcaactgtaaaggaggatggattctgcaatgagcagtaagatcggaagagcgtcgtgtagggaaagagtcattgcgtgaaccga

SEQ ID NO: 740 - DNA, 91 bp, Artificial Sequence
  Probe
aggaccggatcaactgacttgctgtgaacgagccgttgacacataagatcggaagagcgtcgtgtagggaaagagtcattgcgtgaaccga

SEQ ID NO: 741 - DNA, 90 bp, Artificial Sequence
  Probe
aggaccggatcaacttgacccgttccgctcttgcgcgtatagcgagatcggaagagcgtcgtgtagggaaagagtcattgcgtgaaccga

SEQ ID NO: 742 - DNA, 93 bp, Artificial Sequence
  Probe
aggaccggatcaactcatgacaggtattctgaaaaccgttagatgacagatcggaagagcgtcgtgtagggaaagagtcattgcgtgaaccga

SEQ ID NO: 743 - DNA, 94 bp, Artificial Sequence
  Probe
aggaccggatcaactaaaataaaacctcgcagcaacttgggtcacatcagatcggaagagcgtcgtgtagggaaagagtcattgcgtgaaccga

SEQ ID NO: 744 - DNA, 92 bp, Artificial Sequence
  Probe
aggaccggatcaactttttgtcgtgggcgagccaaatctcgatgcaagatcggaagagcgtcgtgtagggaaagagtcattgcgtgaaccga

SEQ ID NO: 745 - DNA, 91 bp, Artificial Sequence
  Probe
aggaccggatcaactgtgtattggctaccagcctcagtcagctatagatcggaagagcgtcgtgtagggaaagagtcattgcgtgaaccga

SEQ ID NO: 746 - DNA, 90 bp, Artificial Sequence
  Probe
aggaccggatcaactgcttccatggatctggaccgggctactgaagatcggaagagcgtcgtgtagggaaagagtcattgcgtgaaccga

SEQ ID NO: 747 - DNA, 91 bp, Artificial Sequence
  Probe
aggaccggatcaactcagtgaccctcgctttcgaacctgcgcgacagatcggaagagcgtcgtgtagggaaagagtcattgcgtgaaccga

SEQ ID NO: 748 - DNA, 92 bp, Artificial Sequence
  Probe
aggaccggatcaactgaatggctgcgatcaagattgggtcgctatcagatcggaagagcgtcgtgtagggaaagagtcattgcgtgaaccga

SEQ ID NO: 749 - DNA, 93 bp, Artificial Sequence
  Probe
aggaccggatcaacttgctgctggtgagctaataatcttatagtcatagatcggaagagcgtcgtgtagggaaagagtcattgcgtgaaccga

SEQ ID NO: 750 - DNA, 92 bp, Artificial Sequence
  Probe
aggaccggatcaactacctctggagtattctgaagtggtgagtacgagatcggaagagcgtcgtgtagggaaagagtcattgcgtgaaccga

SEQ ID NO: 751 - DNA, 92 bp, Artificial Sequence
  Probe
aggaccggatcaacttaccctttccttagggacgacagtgctcgcaagatcggaagagcgtcgtgtagggaaagagtcattgcgtgaaccga

SEQ ID NO: 752 - DNA, 92 bp, Artificial Sequence
  Probe
aggaccggatcaacttccactagggtagatcactctgcactcactgagatcggaagagcgtcgtgtagggaaagagtcattgcgtgaaccga

SEQ ID NO: 753 - DNA, 93 bp, Artificial Sequence
  Probe
aggaccggatcaactgataaacaaagagctgcaatggccatgcatctagatcggaagagcgtcgtgtagggaaagagtcattgcgtgaaccga

SEQ ID NO: 754 - DNA, 92 bp, Artificial Sequence
  Probe
aggaccggatcaactacagatacctctttagctgcacctactctgaagatcggaagagcgtcgtgtagggaaagagtcattgcgtgaaccga

SEQ ID NO: 755 - DNA, 92 bp, Artificial Sequence
  Probe
aggaccggatcaactgggagattcaggtaagtgtgtgcacgtagcgagatcggaagagcgtcgtgtagggaaagagtcattgcgtgaaccga

SEQ ID NO: 756 - DNA, 93 bp, Artificial Sequence
  Probe
aggaccggatcaactttcctgaagtaaaagttcctcagcctctacgcagatcggaagagcgtcgtgtagggaaagagtcattgcgtgaaccga

SEQ ID NO: 757 - DNA, 89 bp, Artificial Sequence
  Probe
aggaccggatcaactcaggccagcgtccctgaccagctcgtagagatcggaagagcgtcgtgtagggaaagagtcattgcgtgaaccga

SEQ ID NO: 758 - DNA, 92 bp, Artificial Sequence
  Probe
aggaccggatcaactatttcctctgcactcagtccagcatgactctagatcggaagagcgtcgtgtagggaaagagtcattgcgtgaaccga

SEQ ID NO: 759 - DNA, 92 bp, Artificial Sequence
  Probe
aggaccggatcaactgtttggatcctctgtaactgcgtgtgagagaagatcggaagagcgtcgtgtagggaaagagtcattgcgtgaaccga

SEQ ID NO: 760 - DNA, 90 bp, Artificial Sequence
  Probe
aggaccggatcaactcgcggcatcgatggctacgagagctcataagatcggaagagcgtcgtgtagggaaagagtcattgcgtgaaccga

SEQ ID NO: 761 - DNA, 94 bp, Artificial Sequence
  Probe
aggaccggatcaactcgtcatataaaagggattaagaggccgtagcagagatcggaagagcgtcgtgtagggaaagagtcattgcgtgaaccga

SEQ ID NO: 762 - DNA, 94 bp, Artificial Sequence
  Probe
aggaccggatcaactaagcatatttctttctccgagtgattacatgtcagatcggaagagcgtcgtgtagggaaagagtcattgcgtgaaccga

SEQ ID NO: 763 - DNA, 92 bp, Artificial Sequence
  Probe
aggaccggatcaactacacgatataccggcgacgaataagctcacgagatcggaagagcgtcgtgtagggaaagagtcattgcgtgaaccga

SEQ ID NO: 764 - DNA, 93 bp, Artificial Sequence
  Probe
aggaccggatcaactccatcaacatattgctgcagtgtcgagcagctagatcggaagagcgtcgtgtagggaaagagtcattgcgtgaaccga

SEQ ID NO: 765 - DNA, 93 bp, Artificial Sequence
  Probe
aggaccggatcaacttgcttgggtttaacgtcagaaacatcagagatagatcggaagagcgtcgtgtagggaaagagtcattgcgtgaaccga

SEQ ID NO: 766 - DNA, 94 bp, Artificial Sequence
  Probe
aggaccggatcaactaatactccttgagatggaacagaagcgagctagagatcggaagagcgtcgtgtagggaaagagtcattgcgtgaaccga

SEQ ID NO: 767 - DNA, 90 bp, Artificial Sequence
  Probe
aggaccggatcaacttctcctcccctagtggctgagtgcacacgagatcggaagagcgtcgtgtagggaaagagtcattgcgtgaaccga

SEQ ID NO: 768 - DNA, 93 bp, Artificial Sequence
  Probe
aggaccggatcaactaacaaaaacgtctttattgccggcatcgagtcagatcggaagagcgtcgtgtagggaaagagtcattgcgtgaaccga

SEQ ID NO: 769 - DNA, 95 bp, Artificial Sequence
  Probe
aggaccggatcaactgagaatgatcagtaaatgcaataagcgtgacataagatcggaagagcgtcgtgtagggaaagagtcattgcgtgaaccga

SEQ ID NO: 770 - DNA, 94 bp, Artificial Sequence
  Probe
aggaccggatcaactaacataccatgcaaatgtgttgacgcacgagcgagatcggaagagcgtcgtgtagggaaagagtcattgcgtgaaccga

SEQ ID NO: 771 - DNA, 92 bp, Artificial Sequence
  Probe
aggaccggatcaactggcagtcagaatctttgatgcgccatgtcatagatcggaagagcgtcgtgtagggaaagagtcattgcgtgaaccga

SEQ ID NO: 772 - DNA, 91 bp, Artificial Sequence
  Probe
aggaccggatcaactgttggacgttttgaagtcccggtatctctgagatcggaagagcgtcgtgtagggaaagagtcattgcgtgaaccga

SEQ ID NO: 773 - DNA, 90 bp, Artificial Sequence
  Probe
aggaccggatcaactggtgagcacggttccgtgatcctgagtgtagatcggaagagcgtcgtgtagggaaagagtcattgcgtgaaccga

SEQ ID NO: 774 - DNA, 92 bp, Artificial Sequence
  Probe
aggaccggatcaactcttttctggatcacaccgactaggtagatatagatcggaagagcgtcgtgtagggaaagagtcattgcgtgaaccga

SEQ ID NO: 775 - DNA, 91 bp, Artificial Sequence
  Probe
aggaccggatcaactggtggactctctctcctttggccactagtgagatcggaagagcgtcgtgtagggaaagagtcattgcgtgaaccga

SEQ ID NO: 776 - DNA, 93 bp, Artificial Sequence
  Probe
aggaccggatcaactgatagcgcaataattaaaccggcgcgacgtctagatcggaagagcgtcgtgtagggaaagagtcattgcgtgaaccga

SEQ ID NO: 777 - DNA, 91 bp, Artificial Sequence
  Probe
aggaccggatcaactgcaacaagccacgacctcttgactagtagcagatcggaagagcgtcgtgtagggaaagagtcattgcgtgaaccga

SEQ ID NO: 778 - DNA, 92 bp, Artificial Sequence
  Probe
aggaccggatcaactgacctgccaacacaaaatagtgcgcgtctgcagatcggaagagcgtcgtgtagggaaagagtcattgcgtgaaccga

SEQ ID NO: 779 - DNA, 92 bp, Artificial Sequence
  Probe
aggaccggatcaactctctacttgcgaacacgttctgttagtcactagatcggaagagcgtcgtgtagggaaagagtcattgcgtgaaccga

SEQ ID NO: 780 - DNA, 93 bp, Artificial Sequence
  Probe
aggaccggatcaacttagacacatgtaataaggccaccctacatcgtagatcggaagagcgtcgtgtagggaaagagtcattgcgtgaaccga

SEQ ID NO: 781 - DNA, 92 bp, Artificial Sequence
  Probe
aggaccggatcaactaattagaacgaaccaagctgcgcctgatcatagatcggaagagcgtcgtgtagggaaagagtcattgcgtgaaccga

SEQ ID NO: 782 - DNA, 93 bp, Artificial Sequence
  Probe
aggaccggatcaactcatttgagtggtcgtttgtttcgtgatcactaagatcggaagagcgtcgtgtagggaaagagtcattgcgtgaaccga

SEQ ID NO: 783 - DNA, 91 bp, Artificial Sequence
  Probe
aggaccggatcaactagctgagccggtctagaaaccggcgactcgagatcggaagagcgtcgtgtagggaaagagtcattgcgtgaaccga

SEQ ID NO: 784 - DNA, 93 bp, Artificial Sequence
  Probe
aggaccggatcaactgcccctttattttgatgtttgcgcctagatctagatcggaagagcgtcgtgtagggaaagagtcattgcgtgaaccga

SEQ ID NO: 785 - DNA, 92 bp, Artificial Sequence
  Probe
aggaccggatcaactcatcatagcactgtcagcatggaatcgcgctagatcggaagagcgtcgtgtagggaaagagtcattgcgtgaaccga

SEQ ID NO: 786 - DNA, 93 bp, Artificial Sequence
  Probe
aggaccggatcaactctaatgactcttgcaaggtggaacactgatatagatcggaagagcgtcgtgtagggaaagagtcattgcgtgaaccga

SEQ ID NO: 787 - DNA, 94 bp, Artificial Sequence
  Probe
aggaccggatcaactataaactaacgctcaattgcgtctcatctgtgcagatcggaagagcgtcgtgtagggaaagagtcattgcgtgaaccga

SEQ ID NO: 788 - DNA, 92 bp, Artificial Sequence
  Probe
aggaccggatcaactagagaggggctagaaaggtagaaagtgtgcaagatcggaagagcgtcgtgtagggaaagagtcattgcgtgaaccga

SEQ ID NO: 789 - DNA, 92 bp, Artificial Sequence
  Probe
aggaccggatcaactcgtgatttcgcacaacgttacagcactacacagatcggaagagcgtcgtgtagggaaagagtcattgcgtgaaccga

SEQ ID NO: 790 - DNA, 92 bp, Artificial Sequence
  Probe
aggaccggatcaactccgtccaaataacatcagaggcccacgatatagatcggaagagcgtcgtgtagggaaagagtcattgcgtgaaccga

SEQ ID NO: 791 - DNA, 93 bp, Artificial Sequence
  Probe
aggaccggatcaactgcttcggcatataagaccaaactgcacgctagagatcggaagagcgtcgtgtagggaaagagtcattgcgtgaaccga

SEQ ID NO: 792 - DNA, 92 bp, Artificial Sequence
  Probe
aggaccggatcaactgcctctacttttccttgctcgtaatcgcataagatcggaagagcgtcgtgtagggaaagagtcattgcgtgaaccga

SEQ ID NO: 793 - DNA, 93 bp, Artificial Sequence
  Probe
aggaccggatcaactttcttgtccttgttttcgattgccgcatcgctagatcggaagagcgtcgtgtagggaaagagtcattgcgtgaaccga

SEQ ID NO: 794 - DNA, 93 bp, Artificial Sequence
  Probe
aggaccggatcaacttgttctattccagttggcatggtatcatctacagatcggaagagcgtcgtgtagggaaagagtcattgcgtgaaccga

SEQ ID NO: 795 - DNA, 94 bp, Artificial Sequence
  Probe
aggaccggatcaacttggaaactaacattctatcggtaggtgcactcaagatcggaagagcgtcgtgtagggaaagagtcattgcgtgaaccga

SEQ ID NO: 796 - DNA, 91 bp, Artificial Sequence
  Probe
aggaccggatcaactcacccgattcagaggtgcatcagcgatgtaagatcggaagagcgtcgtgtagggaaagagtcattgcgtgaaccga

SEQ ID NO: 797 - DNA, 94 bp, Artificial Sequence
  Probe
aggaccggatcaactgtagagacagttaagttcagttcattatagcagagatcggaagagcgtcgtgtagggaaagagtcattgcgtgaaccga

SEQ ID NO: 798 - DNA, 91 bp, Artificial Sequence
  Probe
aggaccggatcaacttggcgaagatggcaagagcagctgcgagcaagatcggaagagcgtcgtgtagggaaagagtcattgcgtgaaccga

SEQ ID NO: 799 - DNA, 93 bp, Artificial Sequence
  Probe
aggaccggatcaactattgatggagagaagatacatgggagactagaagatcggaagagcgtcgtgtagggaaagagtcattgcgtgaaccga

SEQ ID NO: 800 - DNA, 92 bp, Artificial Sequence
  Probe
aggaccggatcaactaagatcgaaattagtcccggtggtcactcacagatcggaagagcgtcgtgtagggaaagagtcattgcgtgaaccga

SEQ ID NO: 801 - DNA, 91 bp, Artificial Sequence
  Probe
aggaccggatcaactggatcagcgcgtgaagcattcatcagatgtagatcggaagagcgtcgtgtagggaaagagtcattgcgtgaaccga

SEQ ID NO: 802 - DNA, 92 bp, Artificial Sequence
  Probe
aggaccggatcaactgtttagaatggtcagcttccctgatctgtcaagatcggaagagcgtcgtgtagggaaagagtcattgcgtgaaccga

SEQ ID NO: 803 - DNA, 92 bp, Artificial Sequence
  Probe
aggaccggatcaacttgtgctcactggttcttggttcgcagtactgagatcggaagagcgtcgtgtagggaaagagtcattgcgtgaaccga

SEQ ID NO: 804 - DNA, 92 bp, Artificial Sequence
  Probe
aggaccggatcaactctacatccttagatgtggcgacatcagtgagagatcggaagagcgtcgtgtagggaaagagtcattgcgtgaaccga

SEQ ID NO: 805 - DNA, 93 bp, Artificial Sequence
  Probe
aggaccggatcaacttacgttcaaggctgactggaatttacgcatcaagatcggaagagcgtcgtgtagggaaagagtcattgcgtgaaccga

SEQ ID NO: 806 - DNA, 93 bp, Artificial Sequence
  Probe
aggaccggatcaacttctcccatcgaaaaatcactatcccgtctcatagatcggaagagcgtcgtgtagggaaagagtcattgcgtgaaccga

SEQ ID NO: 807 - DNA, 95 bp, Artificial Sequence
  Probe
aggaccggatcaacttgctttattttgatagctgcaacttggactcagaagatcggaagagcgtcgtgtagggaaagagtcattgcgtgaaccga

SEQ ID NO: 808 - DNA, 93 bp, Artificial Sequence
  Probe
aggaccggatcaactctgccttgttcagtctgctaattatacagagtagatcggaagagcgtcgtgtagggaaagagtcattgcgtgaaccga

SEQ ID NO: 809 - DNA, 94 bp, Artificial Sequence
  Probe
aggaccggatcaactaacaatgaagttgcagcaaacacaaagtcacgcagatcggaagagcgtcgtgtagggaaagagtcattgcgtgaaccga

SEQ ID NO: 810 - DNA, 92 bp, Artificial Sequence
  Probe
aggaccggatcaactcgtttctgctaggaggaccatactctgctagagatcggaagagcgtcgtgtagggaaagagtcattgcgtgaaccga

SEQ ID NO: 811 - DNA, 94 bp, Artificial Sequence
  Probe
aggaccggatcaactgccacttacataatcatagctaatcatctctcgagatcggaagagcgtcgtgtagggaaagagtcattgcgtgaaccga

SEQ ID NO: 812 - DNA, 93 bp, Artificial Sequence
  Probe
aggaccggatcaactagaggcaatattctacacgtgcaagagacacgagatcggaagagcgtcgtgtagggaaagagtcattgcgtgaaccga

SEQ ID NO: 813 - DNA, 90 bp, Artificial Sequence
  Probe
aggaccggatcaactgagcgccggttttggaaccagtgtagctcagatcggaagagcgtcgtgtagggaaagagtcattgcgtgaaccga

SEQ ID NO: 814 - DNA, 93 bp, Artificial Sequence
  Probe
aggaccggatcaactgtgctttcggagttattgtttggagctcacgtagatcggaagagcgtcgtgtagggaaagagtcattgcgtgaaccga

SEQ ID NO: 815 - DNA, 89 bp, Artificial Sequence
  Probe
aggaccggatcaactggcgaggacgacccgtagcagcgatatgagatcggaagagcgtcgtgtagggaaagagtcattgcgtgaaccga

SEQ ID NO: 816 - DNA, 92 bp, Artificial Sequence
  Probe
aggaccggatcaactagccgtgttgcatcatgcttctactcgagagagatcggaagagcgtcgtgtagggaaagagtcattgcgtgaaccga

SEQ ID NO: 817 - DNA, 93 bp, Artificial Sequence
  Probe
aggaccggatcaacttcttacgatcttgtcaaacagctcgagatgtcagatcggaagagcgtcgtgtagggaaagagtcattgcgtgaaccga

SEQ ID NO: 818 - DNA, 93 bp, Artificial Sequence
  Probe
aggaccggatcaactagagaaacaacagatcagaccatgagcgtgagagatcggaagagcgtcgtgtagggaaagagtcattgcgtgaaccga

SEQ ID NO: 819 - DNA, 93 bp, Artificial Sequence
  Probe
aggaccggatcaactcttggcgctgctcttgtattttttgacgctatagatcggaagagcgtcgtgtagggaaagagtcattgcgtgaaccga

SEQ ID NO: 820 - DNA, 91 bp, Artificial Sequence
  Probe
aggaccggatcaactggcactcatgcatgatcctcctcgactgcgagatcggaagagcgtcgtgtagggaaagagtcattgcgtgaaccga

SEQ ID NO: 821 - DNA, 92 bp, Artificial Sequence
  Probe
aggaccggatcaactactagtgcttgccagtattccagtactgatgagatcggaagagcgtcgtgtagggaaagagtcattgcgtgaaccga

SEQ ID NO: 822 - DNA, 92 bp, Artificial Sequence
  Probe
aggaccggatcaactttgcacctgcagcctatctattcactgtacaagatcggaagagcgtcgtgtagggaaagagtcattgcgtgaaccga

SEQ ID NO: 823 - DNA, 93 bp, Artificial Sequence
  Probe
aggaccggatcaacttccgatgtgctaaattcatcacccgtagtacaagatcggaagagcgtcgtgtagggaaagagtcattgcgtgaaccga

SEQ ID NO: 824 - DNA, 94 bp, Artificial Sequence
  Probe
aggaccggatcaactctaccttttatgtccttactactgcgacacgacagatcggaagagcgtcgtgtagggaaagagtcattgcgtgaaccga

SEQ ID NO: 825 - DNA, 93 bp, Artificial Sequence
  Probe
aggaccggatcaacttatttggatgattctgagtggggcgcgcgtgcagatcggaagagcgtcgtgtagggaaagagtcattgcgtgaaccga

SEQ ID NO: 826 - DNA, 93 bp, Artificial Sequence
  Probe
aggaccggatcaactaaggagttagagagacaaggactacacgtgcaagatcggaagagcgtcgtgtagggaaagagtcattgcgtgaaccga

SEQ ID NO: 827 - DNA, 91 bp, Artificial Sequence
  Probe
aggaccggatcaactcagcctggggaacctagttttgctactataagatcggaagagcgtcgtgtagggaaagagtcattgcgtgaaccga

SEQ ID NO: 828 - DNA, 93 bp, Artificial Sequence
  Probe
aggaccggatcaactcgcagcaatacgtctcaaaatctactgcgtcgagatcggaagagcgtcgtgtagggaaagagtcattgcgtgaaccga

SEQ ID NO: 829 - DNA, 92 bp, Artificial Sequence
  Probe
aggaccggatcaacttagttccattagcagcctgtggaagtatataagatcggaagagcgtcgtgtagggaaagagtcattgcgtgaaccga

SEQ ID NO: 830 - DNA, 92 bp, Artificial Sequence
  Probe
aggaccggatcaactgtccatcttccatactcccactttgacagtcagatcggaagagcgtcgtgtagggaaagagtcattgcgtgaaccga

SEQ ID NO: 831 - DNA, 91 bp, Artificial Sequence
  Probe
aggaccggatcaactgcgacagctttgcgagtccttcatcgcagcagatcggaagagcgtcgtgtagggaaagagtcattgcgtgaaccga

SEQ ID NO: 832 - DNA, 93 bp, Artificial Sequence
  Probe
aggaccggatcaactttcaccattcgccaaactatagcaacactctgagatcggaagagcgtcgtgtagggaaagagtcattgcgtgaaccga

SEQ ID NO: 833 - DNA, 93 bp, Artificial Sequence
  Probe
aggaccggatcaactaataagcagctgtcaaatcagcacctgctgtaagatcggaagagcgtcgtgtagggaaagagtcattgcgtgaaccga

SEQ ID NO: 834 - DNA, 91 bp, Artificial Sequence
  Probe
aggaccggatcaactgtggacaagggtacagggaagagagcacacagatcggaagagcgtcgtgtagggaaagagtcattgcgtgaaccga

SEQ ID NO: 835 - DNA, 92 bp, Artificial Sequence
  Probe
aggaccggatcaactaagcagctcagagttggattcctgagctctgagatcggaagagcgtcgtgtagggaaagagtcattgcgtgaaccga

SEQ ID NO: 836 - DNA, 91 bp, Artificial Sequence
  Probe
aggaccggatcaactgaccgtctaaacagctgctctcgtatcacgagatcggaagagcgtcgtgtagggaaagagtcattgcgtgaaccga

SEQ ID NO: 837 - DNA, 93 bp, Artificial Sequence
  Probe
aggaccggatcaactgatgtgaggtaatctgaatacagcgctgactaagatcggaagagcgtcgtgtagggaaagagtcattgcgtgaaccga

SEQ ID NO: 838 - DNA, 94 bp, Artificial Sequence
  Probe
aggaccggatcaacttgttcctttcatatggaaaaacagctctgtactagatcggaagagcgtcgtgtagggaaagagtcattgcgtgaaccga

SEQ ID NO: 839 - DNA, 92 bp, Artificial Sequence
  Probe
aggaccggatcaactcaccgaaagatttggacaggagtgagcgcagagatcggaagagcgtcgtgtagggaaagagtcattgcgtgaaccga

SEQ ID NO: 840 - DNA, 93 bp, Artificial Sequence
  Probe
aggaccggatcaactggaatagaaaatcgcagcatcactacgactgtagatcggaagagcgtcgtgtagggaaagagtcattgcgtgaaccga

SEQ ID NO: 841 - DNA, 91 bp, Artificial Sequence
  Probe
aggaccggatcaactgagattgcgagatgatgagccctcgagtgtagatcggaagagcgtcgtgtagggaaagagtcattgcgtgaaccga

SEQ ID NO: 842 - DNA, 90 bp, Artificial Sequence
  Probe
aggaccggatcaactctctggcacctgcagcacttcgctctacaagatcggaagagcgtcgtgtagggaaagagtcattgcgtgaaccga

SEQ ID NO: 843 - DNA, 91 bp, Artificial Sequence
  Probe
aggaccggatcaacttggaataactggtctctgccggcatactatagatcggaagagcgtcgtgtagggaaagagtcattgcgtgaaccga

SEQ ID NO: 844 - DNA, 92 bp, Artificial Sequence
  Probe
aggaccggatcaactcggcagcacctacatcatactaagcgatgcgagatcggaagagcgtcgtgtagggaaagagtcattgcgtgaaccga

SEQ ID NO: 845 - DNA, 92 bp, Artificial Sequence
  Probe
aggaccggatcaactagtttgacgcttgcattgccatgactacgtaagatcggaagagcgtcgtgtagggaaagagtcattgcgtgaaccga

SEQ ID NO: 846 - DNA, 92 bp, Artificial Sequence
  Probe
aggaccggatcaacttctctgtttgaatccagctgtgcacgtgtgcagatcggaagagcgtcgtgtagggaaagagtcattgcgtgaaccga

SEQ ID NO: 847 - DNA, 92 bp, Artificial Sequence
  Probe
aggaccggatcaactgataatggtccggtggctcattgatatctctagatcggaagagcgtcgtgtagggaaagagtcattgcgtgaaccga

SEQ ID NO: 848 - DNA, 93 bp, Artificial Sequence
  Probe
aggaccggatcaactggggacattatcaacatgatgtgggtgagtcgagatcggaagagcgtcgtgtagggaaagagtcattgcgtgaaccga

SEQ ID NO: 849 - DNA, 92 bp, Artificial Sequence
  Probe
aggaccggatcaactgtgatgagtgtttcgcgaaccaacgcagcgtagatcggaagagcgtcgtgtagggaaagagtcattgcgtgaaccga

SEQ ID NO: 850 - DNA, 91 bp, Artificial Sequence
  Probe
aggaccggatcaactcatgtaccctgactacccttgctctgtgatagatcggaagagcgtcgtgtagggaaagagtcattgcgtgaaccga

SEQ ID NO: 851 - DNA, 91 bp, Artificial Sequence
  Probe
aggaccggatcaactgctgttagctaggctgcttgtgatgtatatagatcggaagagcgtcgtgtagggaaagagtcattgcgtgaaccga

SEQ ID NO: 852 - DNA, 94 bp, Artificial Sequence
  Probe
aggaccggatcaactgcattttgttgtgcttgaacatgaaatcactcaagatcggaagagcgtcgtgtagggaaagagtcattgcgtgaaccga

SEQ ID NO: 853 - DNA, 90 bp, Artificial Sequence
  Probe
aggaccggatcaactttggtgtccagcttgggggcagacgatctagatcggaagagcgtcgtgtagggaaagagtcattgcgtgaaccga

SEQ ID NO: 854 - DNA, 94 bp, Artificial Sequence
  Probe
aggaccggatcaacttccatttactgatacttgtgagcttgtatgactagatcggaagagcgtcgtgtagggaaagagtcattgcgtgaaccga

SEQ ID NO: 855 - DNA, 92 bp, Artificial Sequence
  Probe
aggaccggatcaactcaaccgatgtgcattgaacatgggctcgctaagatcggaagagcgtcgtgtagggaaagagtcattgcgtgaaccga

SEQ ID NO: 856 - DNA, 93 bp, Artificial Sequence
  Probe
aggaccggatcaactggtgaaagatgcttacagctcatcgcatacgtagatcggaagagcgtcgtgtagggaaagagtcattgcgtgaaccga

SEQ ID NO: 857 - DNA, 93 bp, Artificial Sequence
  Probe
aggaccggatcaactttgtcagattgcctagatgttagctgctgcatagatcggaagagcgtcgtgtagggaaagagtcattgcgtgaaccga

SEQ ID NO: 858 - DNA, 93 bp, Artificial Sequence
  Probe
aggaccggatcaactcagttgttgattcaactctgcgtgcactcataagatcggaagagcgtcgtgtagggaaagagtcattgcgtgaaccga

SEQ ID NO: 859 - DNA, 92 bp, Artificial Sequence
  Probe
aggaccggatcaactgacaggccctgtacctattgatgcagtctacagatcggaagagcgtcgtgtagggaaagagtcattgcgtgaaccga

SEQ ID NO: 860 - DNA, 94 bp, Artificial Sequence
  Probe
aggaccggatcaactaactaaatttcttgccaacctgcaggagtagcgagatcggaagagcgtcgtgtagggaaagagtcattgcgtgaaccga

SEQ ID NO: 861 - DNA, 94 bp, Artificial Sequence
  Probe
aggaccggatcaacttttttcacagttgcctgctttttggcagactgtagatcggaagagcgtcgtgtagggaaagagtcattgcgtgaaccga

SEQ ID NO: 862 - DNA, 92 bp, Artificial Sequence
  Probe
aggaccggatcaactgtaggccagtctgttacagacaaacgcgtctagatcggaagagcgtcgtgtagggaaagagtcattgcgtgaaccga

SEQ ID NO: 863 - DNA, 92 bp, Artificial Sequence
  Probe
aggaccggatcaacttatccaagcttccaaggtgaggtagatcgatagatcggaagagcgtcgtgtagggaaagagtcattgcgtgaaccga

SEQ ID NO: 864 - DNA, 92 bp, Artificial Sequence
  Probe
aggaccggatcaactgttccacatggagtgaacagaactgcagtacagatcggaagagcgtcgtgtagggaaagagtcattgcgtgaaccga

SEQ ID NO: 865 - DNA, 91 bp, Artificial Sequence
  Probe
aggaccggatcaactcagagcttgaaggctacttgggtcgagcacagatcggaagagcgtcgtgtagggaaagagtcattgcgtgaaccga

SEQ ID NO: 866 - DNA, 93 bp, Artificial Sequence
  Probe
aggaccggatcaactatcagcgaaggaaatatcaggtactactgacaagatcggaagagcgtcgtgtagggaaagagtcattgcgtgaaccga

SEQ ID NO: 867 - DNA, 92 bp, Artificial Sequence
  Probe
aggaccggatcaactcaggaatttgtccctgatgagcgtgatgctcagatcggaagagcgtcgtgtagggaaagagtcattgcgtgaaccga

SEQ ID NO: 868 - DNA, 91 bp, Artificial Sequence
  Probe
aggaccggatcaacttgccgcaaatgatgaggcctggcgtctcgaagatcggaagagcgtcgtgtagggaaagagtcattgcgtgaaccga

SEQ ID NO: 869 - DNA, 92 bp, Artificial Sequence
  Probe
aggaccggatcaactcacgatgtagtttcagtgtgctgtcgcatcgagatcggaagagcgtcgtgtagggaaagagtcattgcgtgaaccga

SEQ ID NO: 870 - DNA, 92 bp, Artificial Sequence
  Probe
aggaccggatcaactaatggacgcgagatcacgagtacctgatataagatcggaagagcgtcgtgtagggaaagagtcattgcgtgaaccga

SEQ ID NO: 871 - DNA, 93 bp, Artificial Sequence
  Probe
aggaccggatcaactataacagcggacaacacgatgtacatatgcatagatcggaagagcgtcgtgtagggaaagagtcattgcgtgaaccga

SEQ ID NO: 872 - DNA, 91 bp, Artificial Sequence
  Probe
aggaccggatcaactgcatgtgactgctgcctgactaagacgacaagatcggaagagcgtcgtgtagggaaagagtcattgcgtgaaccga

SEQ ID NO: 873 - DNA, 92 bp, Artificial Sequence
  Probe
aggaccggatcaactgatgtgttattagccctggctgcgtcagtacagatcggaagagcgtcgtgtagggaaagagtcattgcgtgaaccga

SEQ ID NO: 874 - DNA, 93 bp, Artificial Sequence
  Probe
aggaccggatcaactaatgttacagcagataaatccgcggtgctagtagatcggaagagcgtcgtgtagggaaagagtcattgcgtgaaccga

SEQ ID NO: 875 - DNA, 91 bp, Artificial Sequence
  Probe
aggaccggatcaactaaaggctggtgtctgagaaggcctgacgtaagatcggaagagcgtcgtgtagggaaagagtcattgcgtgaaccga

SEQ ID NO: 876 - DNA, 94 bp, Artificial Sequence
  Probe
aggaccggatcaacttgcataccttccaatgaaagctatagtctcgatagatcggaagagcgtcgtgtagggaaagagtcattgcgtgaaccga

SEQ ID NO: 877 - DNA, 94 bp, Artificial Sequence
  Probe
aggaccggatcaacttacaataagcaaacacaaatcccgggacgtagaagatcggaagagcgtcgtgtagggaaagagtcattgcgtgaaccga

SEQ ID NO: 878 - DNA, 92 bp, Artificial Sequence
  Probe
aggaccggatcaactagtaatcctcctcagctagtctgcgacatgcagatcggaagagcgtcgtgtagggaaagagtcattgcgtgaaccga

SEQ ID NO: 879 - DNA, 91 bp, Artificial Sequence
  Probe
aggaccggatcaactcacccttacccgggaactaagcacacgctaagatcggaagagcgtcgtgtagggaaagagtcattgcgtgaaccga

SEQ ID NO: 880 - DNA, 94 bp, Artificial Sequence
  Probe
aggaccggatcaacttctaatcaatcctagttaccatggctagtgctcagatcggaagagcgtcgtgtagggaaagagtcattgcgtgaaccga

SEQ ID NO: 881 - DNA, 92 bp, Artificial Sequence
  Probe
aggaccggatcaactttgcgaataacgcatctgctgggcgatcgagagatcggaagagcgtcgtgtagggaaagagtcattgcgtgaaccga

SEQ ID NO: 882 - DNA, 93 bp, Artificial Sequence
  Probe
aggaccggatcaacttgataaactgtaacgcataccggtctcacgagagatcggaagagcgtcgtgtagggaaagagtcattgcgtgaaccga

SEQ ID NO: 883 - DNA, 91 bp, Artificial Sequence
  Probe
aggaccggatcaactggaataggggctgcctgtgattgtactctgagatcggaagagcgtcgtgtagggaaagagtcattgcgtgaaccga

SEQ ID NO: 884 - DNA, 94 bp, Artificial Sequence
  Probe
aggaccggatcaactattaagcatggagtgtcatccatacctacatcgagatcggaagagcgtcgtgtagggaaagagtcattgcgtgaaccga

SEQ ID NO: 885 - DNA, 92 bp, Artificial Sequence
  Probe
aggaccggatcaactcaggatcatgttccatgccatgctgtgcatgagatcggaagagcgtcgtgtagggaaagagtcattgcgtgaaccga

SEQ ID NO: 886 - DNA, 92 bp, Artificial Sequence
  Probe
aggaccggatcaactctcaaagtcatacaccgaagcgcgtgcacgtagatcggaagagcgtcgtgtagggaaagagtcattgcgtgaaccga

SEQ ID NO: 887 - DNA, 92 bp, Artificial Sequence
  Probe
aggaccggatcaactgctatctgcagtcctagtcgttcgcacagagagatcggaagagcgtcgtgtagggaaagagtcattgcgtgaaccga

SEQ ID NO: 888 - DNA, 93 bp, Artificial Sequence
  Probe
aggaccggatcaacttagttgctgtacttgttgagctgtcatgcgatagatcggaagagcgtcgtgtagggaaagagtcattgcgtgaaccga

SEQ ID NO: 889 - DNA, 94 bp, Artificial Sequence
  Probe
aggaccggatcaacttataccctcagcttatatgtgtagttctgatacagatcggaagagcgtcgtgtagggaaagagtcattgcgtgaaccga

SEQ ID NO: 890 - DNA, 94 bp, Artificial Sequence
  Probe
aggaccggatcaactgtttgtgtgtttatgtgatgcgaatgcgatcagagatcggaagagcgtcgtgtagggaaagagtcattgcgtgaaccga

SEQ ID NO: 891 - DNA, 92 bp, Artificial Sequence
  Probe
aggaccggatcaactgctacaaatggcttcagcagtgtgcgcacatagatcggaagagcgtcgtgtagggaaagagtcattgcgtgaaccga

SEQ ID NO: 892 - DNA, 93 bp, Artificial Sequence
  Probe
aggaccggatcaactgctgcgattattttgtgtggtcagagatctgtagatcggaagagcgtcgtgtagggaaagagtcattgcgtgaaccga

SEQ ID NO: 893 - DNA, 94 bp, Artificial Sequence
  Probe
aggaccggatcaactgacttttgatttgcttccagtaaaggatcgtgcagatcggaagagcgtcgtgtagggaaagagtcattgcgtgaaccga

SEQ ID NO: 894 - DNA, 92 bp, Artificial Sequence
  Probe
aggaccggatcaacttcatgtgatgtgcaggaacctgaacgcgtgaagatcggaagagcgtcgtgtagggaaagagtcattgcgtgaaccga

SEQ ID NO: 895 - DNA, 90 bp, Artificial Sequence
  Probe
aggaccggatcaactatgacaccgaggagggcatcgcgcgcgcaagatcggaagagcgtcgtgtagggaaagagtcattgcgtgaaccga

SEQ ID NO: 896 - DNA, 95 bp, Artificial Sequence
  Probe
aggaccggatcaactatttgatcgtaattagttagctgaccgtgatcacagatcggaagagcgtcgtgtagggaaagagtcattgcgtgaaccga

SEQ ID NO: 897 - DNA, 93 bp, Artificial Sequence
  Probe
aggaccggatcaactttgttttgttggtgaagcaacctggtgagctcagatcggaagagcgtcgtgtagggaaagagtcattgcgtgaaccga

SEQ ID NO: 898 - DNA, 93 bp, Artificial Sequence
  Probe
aggaccggatcaactgcgcaatcaaagtcaaaacctagccgcgactgagatcggaagagcgtcgtgtagggaaagagtcattgcgtgaaccga

SEQ ID NO: 899 - DNA, 92 bp, Artificial Sequence
  Probe
aggaccggatcaactgtggctctcttcgagctcaataaatcatgcaagatcggaagagcgtcgtgtagggaaagagtcattgcgtgaaccga

SEQ ID NO: 900 - DNA, 92 bp, Artificial Sequence
  Probe
aggaccggatcaactgatgccattggtgtgaatcaggccgtgtctcagatcggaagagcgtcgtgtagggaaagagtcattgcgtgaaccga

SEQ ID NO: 901 - DNA, 93 bp, Artificial Sequence
  Probe
aggaccggatcaactgaatcccatatagaagaggggaagagagagcaagatcggaagagcgtcgtgtagggaaagagtcattgcgtgaaccga

SEQ ID NO: 902 - DNA, 92 bp, Artificial Sequence
  Probe
aggaccggatcaactcgacacatgccttgctgcaaatgagtacgcaagatcggaagagcgtcgtgtagggaaagagtcattgcgtgaaccga

SEQ ID NO: 903 - DNA, 91 bp, Artificial Sequence
  Probe
aggaccggatcaactgacgacgagtcaactctggaagagcgacgtagatcggaagagcgtcgtgtagggaaagagtcattgcgtgaaccga

SEQ ID NO: 904 - DNA, 91 bp, Artificial Sequence
  Probe
aggaccggatcaactaggctgaccaggtagtaggtctagctctctagatcggaagagcgtcgtgtagggaaagagtcattgcgtgaaccga

SEQ ID NO: 905 - DNA, 92 bp, Artificial Sequence
  Probe
aggaccggatcaactgggatttcctaacactatcgctgagtgtgatagatcggaagagcgtcgtgtagggaaagagtcattgcgtgaaccga

SEQ ID NO: 906 - DNA, 92 bp, Artificial Sequence
  Probe
aggaccggatcaactagaaattacagcaaggccctccgactcacatagatcggaagagcgtcgtgtagggaaagagtcattgcgtgaaccga

SEQ ID NO: 907 - DNA, 93 bp, Artificial Sequence
  Probe
aggaccggatcaactcttctctggaaatggttagcgaacgtgtcatgagatcggaagagcgtcgtgtagggaaagagtcattgcgtgaaccga

SEQ ID NO: 908 - DNA, 91 bp, Artificial Sequence
  Probe
aggaccggatcaactcaacagccatccggcaaaggtgtctcgtcgagatcggaagagcgtcgtgtagggaaagagtcattgcgtgaaccga

SEQ ID NO: 909 - DNA, 92 bp, Artificial Sequence
  Probe
aggaccggatcaactagccatatacagtctcttctggctagagcgtagatcggaagagcgtcgtgtagggaaagagtcattgcgtgaaccga

SEQ ID NO: 910 - DNA, 90 bp, Artificial Sequence
  Probe
aggaccggatcaactcaccacacgctagctgcctctctcacataagatcggaagagcgtcgtgtagggaaagagtcattgcgtgaaccga

SEQ ID NO: 911 - DNA, 93 bp, Artificial Sequence
  Probe
aggaccggatcaacttctggaagatactcgagacattgatagcgtgcagatcggaagagcgtcgtgtagggaaagagtcattgcgtgaaccga

SEQ ID NO: 912 - DNA, 92 bp, Artificial Sequence
  Probe
aggaccggatcaactgctatctctaatgggcagagtgcagtactcgagatcggaagagcgtcgtgtagggaaagagtcattgcgtgaaccga

SEQ ID NO: 913 - DNA, 94 bp, Artificial Sequence
  Probe
aggaccggatcaactcccaaacaaaaagtgaaaaagactgcgtatgatagatcggaagagcgtcgtgtagggaaagagtcattgcgtgaaccga

SEQ ID NO: 914 - DNA, 93 bp, Artificial Sequence
  Probe
aggaccggatcaacttgtcaaagcaagcacagattcatgactctataagatcggaagagcgtcgtgtagggaaagagtcattgcgtgaaccga

SEQ ID NO: 915 - DNA, 90 bp, Artificial Sequence
  Probe
aggaccggatcaactacctcttcgggtgctgcagcacacgctctagatcggaagagcgtcgtgtagggaaagagtcattgcgtgaaccga

SEQ ID NO: 916 - DNA, 94 bp, Artificial Sequence
  Probe
aggaccggatcaactgatgagggataattatgagaaacggtcagacgcagatcggaagagcgtcgtgtagggaaagagtcattgcgtgaaccga

SEQ ID NO: 917 - DNA, 95 bp, Artificial Sequence
  Probe
aggaccggatcaactaaggagtttgattatcttgatgaaagtgagcgctagatcggaagagcgtcgtgtagggaaagagtcattgcgtgaaccga

SEQ ID NO: 918 - DNA, 93 bp, Artificial Sequence
  Probe
aggaccggatcaactatgaccttggaagttgtaacgctgatacgacgagatcggaagagcgtcgtgtagggaaagagtcattgcgtgaaccga

SEQ ID NO: 919 - DNA, 95 bp, Artificial Sequence
  Probe
aggaccggatcaactcatttatcgcagggaataatagttttcgtacgctagatcggaagagcgtcgtgtagggaaagagtcattgcgtgaaccga

SEQ ID NO: 920 - DNA, 94 bp, Artificial Sequence
  Probe
aggaccggatcaactagttcagtgattttgtattgatcccgactagcaagatcggaagagcgtcgtgtagggaaagagtcattgcgtgaaccga

SEQ ID NO: 921 - DNA, 90 bp, Artificial Sequence
  Probe
aggaccggatcaactaccatggcgactgcggagaactatacgcaagatcggaagagcgtcgtgtagggaaagagtcattgcgtgaaccga

SEQ ID NO: 922 - DNA, 92 bp, Artificial Sequence
  Probe
aggaccggatcaactctattccggtgacgtagttctgaactcagagagatcggaagagcgtcgtgtagggaaagagtcattgcgtgaaccga

SEQ ID NO: 923 - DNA, 94 bp, Artificial Sequence
  Probe
aggaccggatcaactggaaagaaatcacatgtattgccagctgtatctagatcggaagagcgtcgtgtagggaaagagtcattgcgtgaaccga

SEQ ID NO: 924 - DNA, 93 bp, Artificial Sequence
  Probe
aggaccggatcaactgattctacttcctttgaccatccaatgtgtcgagatcggaagagcgtcgtgtagggaaagagtcattgcgtgaaccga

SEQ ID NO: 925 - DNA, 94 bp, Artificial Sequence
  Probe
aggaccggatcaactccttttgctaattcagcagcaatacgtcgtcatagatcggaagagcgtcgtgtagggaaagagtcattgcgtgaaccga

SEQ ID NO: 926 - DNA, 92 bp, Artificial Sequence
  Probe
aggaccggatcaacttcaagctctgcatatgtaggctcgctgcgatagatcggaagagcgtcgtgtagggaaagagtcattgcgtgaaccga

SEQ ID NO: 927 - DNA, 91 bp, Artificial Sequence
  Probe
aggaccggatcaactgaggaggaaatagaggaaggcgtcgacgtaagatcggaagagcgtcgtgtagggaaagagtcattgcgtgaaccga

SEQ ID NO: 928 - DNA, 93 bp, Artificial Sequence
  Probe
aggaccggatcaactctgagaaatgcactacatcagcatcagctgctagatcggaagagcgtcgtgtagggaaagagtcattgcgtgaaccga

SEQ ID NO: 929 - DNA, 93 bp, Artificial Sequence
  Probe
aggaccggatcaactgttgttaggttgaccaaccagaactgtagtatagatcggaagagcgtcgtgtagggaaagagtcattgcgtgaaccga

SEQ ID NO: 930 - DNA, 94 bp, Artificial Sequence
  Probe
aggaccggatcaactagtgagagatgcagagcttaataaggatatatgagatcggaagagcgtcgtgtagggaaagagtcattgcgtgaaccga

SEQ ID NO: 931 - DNA, 93 bp, Artificial Sequence
  Probe
aggaccggatcaactgagaagcccatgtcttgctttatatagtcagaagatcggaagagcgtcgtgtagggaaagagtcattgcgtgaaccga

SEQ ID NO: 932 - DNA, 91 bp, Artificial Sequence
  Probe
aggaccggatcaacttcacgcagcaggtcgtatgacttagacacaagatcggaagagcgtcgtgtagggaaagagtcattgcgtgaaccga

SEQ ID NO: 933 - DNA, 92 bp, Artificial Sequence
  Probe
aggaccggatcaactgaagctactaagtcgtcagccaacactatgaagatcggaagagcgtcgtgtagggaaagagtcattgcgtgaaccga

SEQ ID NO: 934 - DNA, 95 bp, Artificial Sequence
  Probe
aggaccggatcaactcaacctatcaatgtttaacaagtaacgtcgagatagatcggaagagcgtcgtgtagggaaagagtcattgcgtgaaccga

SEQ ID NO: 935 - DNA, 95 bp, Artificial Sequence
  Probe
aggaccggatcaactgatgcgatttgcaaaaaattagattgcgtgacgtagatcggaagagcgtcgtgtagggaaagagtcattgcgtgaaccga

SEQ ID NO: 936 - DNA, 92 bp, Artificial Sequence
  Probe
aggaccggatcaactaagtgcagctctcaaagagtcagtgcgagtcagatcggaagagcgtcgtgtagggaaagagtcattgcgtgaaccga

SEQ ID NO: 937 - DNA, 92 bp, Artificial Sequence
  Probe
aggaccggatcaacttgatgtgttaccagctgggaagtctgtgagcagatcggaagagcgtcgtgtagggaaagagtcattgcgtgaaccga

SEQ ID NO: 938 - DNA, 94 bp, Artificial Sequence
  Probe
aggaccggatcaactaaattgtttcctgtgaagcaagtgccacatcgcagatcggaagagcgtcgtgtagggaaagagtcattgcgtgaaccga

SEQ ID NO: 939 - DNA, 93 bp, Artificial Sequence
  Probe
aggaccggatcaactaaggagtacaggtaacagcgaatctgcgcgcgagatcggaagagcgtcgtgtagggaaagagtcattgcgtgaaccga

SEQ ID NO: 940 - DNA, 94 bp, Artificial Sequence
  Probe
aggaccggatcaactaatatataccggaatgtcacccttctacatagcagatcggaagagcgtcgtgtagggaaagagtcattgcgtgaaccga

SEQ ID NO: 941 - DNA, 91 bp, Artificial Sequence
  Probe
aggaccggatcaacttcaccttctctgccatgctgcttgatcgacagatcggaagagcgtcgtgtagggaaagagtcattgcgtgaaccga

SEQ ID NO: 942 - DNA, 93 bp, Artificial Sequence
  Probe
aggaccggatcaactgcttacgtatcaatgtgcagatagtgagctcaagatcggaagagcgtcgtgtagggaaagagtcattgcgtgaaccga

SEQ ID NO: 943 - DNA, 94 bp, Artificial Sequence
  Probe
aggaccggatcaactaaagagaacaatcatcgtcatgttcgatagtgaagatcggaagagcgtcgtgtagggaaagagtcattgcgtgaaccga

SEQ ID NO: 944 - DNA, 92 bp, Artificial Sequence
  Probe
aggaccggatcaactctgttctgtcgtaacttccggtgtagacgatagatcggaagagcgtcgtgtagggaaagagtcattgcgtgaaccga

SEQ ID NO: 945 - DNA, 91 bp, Artificial Sequence
  Probe
aggaccggatcaactggaaagtgccggccattgttggtatcgtgaagatcggaagagcgtcgtgtagggaaagagtcattgcgtgaaccga

SEQ ID NO: 946 - DNA, 93 bp, Artificial Sequence
  Probe
aggaccggatcaacttgcagaatgaagtgctgttgcaaactcacgtcagatcggaagagcgtcgtgtagggaaagagtcattgcgtgaaccga

SEQ ID NO: 947 - DNA, 92 bp, Artificial Sequence
  Probe
aggaccggatcaactgttacttacttccaggggtcgtctacgtatcagatcggaagagcgtcgtgtagggaaagagtcattgcgtgaaccga

SEQ ID NO: 948 - DNA, 94 bp, Artificial Sequence
  Probe
aggaccggatcaactgtaatgttatgctgcctgctttaaagcgtagtaagatcggaagagcgtcgtgtagggaaagagtcattgcgtgaaccga

SEQ ID NO: 949 - DNA, 93 bp, Artificial Sequence
  Probe
aggaccggatcaactgaggaaatagattgtctgtccagcgagcgagcagatcggaagagcgtcgtgtagggaaagagtcattgcgtgaaccga

SEQ ID NO: 950 - DNA, 93 bp, Artificial Sequence
  Probe
aggaccggatcaactatggataaaactgcagcatctgcatcatctcaagatcggaagagcgtcgtgtagggaaagagtcattgcgtgaaccga

SEQ ID NO: 951 - DNA, 93 bp, Artificial Sequence
  Probe
aggaccggatcaactggttgaccaagttgcaattcactcgcatcatgagatcggaagagcgtcgtgtagggaaagagtcattgcgtgaaccga

SEQ ID NO: 952 - DNA, 93 bp, Artificial Sequence
  Probe
aggaccggatcaactgagaatctgactcaaccatgatacatcgtgatagatcggaagagcgtcgtgtagggaaagagtcattgcgtgaaccga

SEQ ID NO: 953 - DNA, 95 bp, Artificial Sequence
  Probe
aggaccggatcaactatctttgtcaaaatacgaaaatgctgatacgagcagatcggaagagcgtcgtgtagggaaagagtcattgcgtgaaccga

SEQ ID NO: 954 - DNA, 91 bp, Artificial Sequence
  Probe
aggaccggatcaactgacaagctcagtatcgtccacggctgcgtaagatcggaagagcgtcgtgtagggaaagagtcattgcgtgaaccga

SEQ ID NO: 955 - DNA, 93 bp, Artificial Sequence
  Probe
aggaccggatcaacttaacctgcatccttgctagttttgagtgagagagatcggaagagcgtcgtgtagggaaagagtcattgcgtgaaccga

SEQ ID NO: 956 - DNA, 94 bp, Artificial Sequence
  Probe
aggaccggatcaactagaaaaataacccccgaaaatctgtacactatcagatcggaagagcgtcgtgtagggaaagagtcattgcgtgaaccga

SEQ ID NO: 957 - DNA, 92 bp, Artificial Sequence
  Probe
aggaccggatcaacttatgctaacccattctccggtctcacgtacgagatcggaagagcgtcgtgtagggaaagagtcattgcgtgaaccga

SEQ ID NO: 958 - DNA, 92 bp, Artificial Sequence
  Probe
aggaccggatcaacttgcgagaggtgaatgtgagtgaggcacactaagatcggaagagcgtcgtgtagggaaagagtcattgcgtgaaccga

SEQ ID NO: 959 - DNA, 92 bp, Artificial Sequence
  Probe
aggaccggatcaactggcacaaatgcagacactgttaggagatcgcagatcggaagagcgtcgtgtagggaaagagtcattgcgtgaaccga

SEQ ID NO: 960 - DNA, 91 bp, Artificial Sequence
  Probe
aggaccggatcaactctgaagctgcacgacatgtcgctactatatagatcggaagagcgtcgtgtagggaaagagtcattgcgtgaaccga

SEQ ID NO: 961 - DNA, 94 bp, Artificial Sequence
  Probe
aggaccggatcaactgagaaggtaagaccaccttaaaattgtcacacaagatcggaagagcgtcgtgtagggaaagagtcattgcgtgaaccga

SEQ ID NO: 962 - DNA, 93 bp, Artificial Sequence
  Probe
aggaccggatcaactttcgctaggttaagacatggagacgctcgtgaagatcggaagagcgtcgtgtagggaaagagtcattgcgtgaaccga

SEQ ID NO: 963 - DNA, 91 bp, Artificial Sequence
  Probe
aggaccggatcaactaggttgtggtcacttgctcgtctctagatgagatcggaagagcgtcgtgtagggaaagagtcattgcgtgaaccga

SEQ ID NO: 964 - DNA, 96 bp, Artificial Sequence
  Probe
aggaccggatcaactatgttaatttctagagtttttcctgttagatgacgagatcggaagagcgtcgtgtagggaaagagtcattgcgtgaaccga

SEQ ID NO: 965 - DNA, 93 bp, Artificial Sequence
  Probe
aggaccggatcaactgagtttggtatgcagtggttgttggtacagtgagatcggaagagcgtcgtgtagggaaagagtcattgcgtgaaccga

SEQ ID NO: 966 - DNA, 91 bp, Artificial Sequence
  Probe
aggaccggatcaactgcaatcgaagctctgcagtggctctatcagagatcggaagagcgtcgtgtagggaaagagtcattgcgtgaaccga

SEQ ID NO: 967 - DNA, 92 bp, Artificial Sequence
  Probe
aggaccggatcaactcctgcatatgcatatgccatgggtgtgacgcagatcggaagagcgtcgtgtagggaaagagtcattgcgtgaaccga

SEQ ID NO: 968 - DNA, 94 bp, Artificial Sequence
  Probe
aggaccggatcaacttaaatgttctgcaaaaggtccgtttactgtatcagatcggaagagcgtcgtgtagggaaagagtcattgcgtgaaccga

SEQ ID NO: 969 - DNA, 92 bp, Artificial Sequence
  Probe
aggaccggatcaactgagcttgacatgctaacaccttcatcatataagatcggaagagcgtcgtgtagggaaagagtcattgcgtgaaccga

SEQ ID NO: 970 - DNA, 91 bp, Artificial Sequence
  Probe
aggaccggatcaactaagccagggactcggatgaactgctatgatagatcggaagagcgtcgtgtagggaaagagtcattgcgtgaaccga

SEQ ID NO: 971 - DNA, 94 bp, Artificial Sequence
  Probe
aggaccggatcaacttttgtcaacttgtcaacatcagagctcgagtcgagatcggaagagcgtcgtgtagggaaagagtcattgcgtgaaccga

SEQ ID NO: 972 - DNA, 91 bp, Artificial Sequence
  Probe
aggaccggatcaactgtatccgtgtcgcttgtagagctatatcgaagatcggaagagcgtcgtgtagggaaagagtcattgcgtgaaccga

SEQ ID NO: 973 - DNA, 94 bp, Artificial Sequence
  Probe
aggaccggatcaactgatcacatcaacgaacttgtaaaccgctcgcgcagatcggaagagcgtcgtgtagggaaagagtcattgcgtgaaccga

SEQ ID NO: 974 - DNA, 91 bp, Artificial Sequence
  Probe
aggaccggatcaactgaagcatgggcctctctcgatccgtgctgtagatcggaagagcgtcgtgtagggaaagagtcattgcgtgaaccga

SEQ ID NO: 975 - DNA, 92 bp, Artificial Sequence
  Probe
aggaccggatcaacttaacatctcgtcggcatagaggcgcacgctgagatcggaagagcgtcgtgtagggaaagagtcattgcgtgaaccga

SEQ ID NO: 976 - DNA, 95 bp, Artificial Sequence
  Probe
aggaccggatcaacttaatatgcagctaacatctcatatcctcagatagagatcggaagagcgtcgtgtagggaaagagtcattgcgtgaaccga

SEQ ID NO: 977 - DNA, 92 bp, Artificial Sequence
  Probe
aggaccggatcaactccggcaattaggtggatgtcataactcgctcagatcggaagagcgtcgtgtagggaaagagtcattgcgtgaaccga

SEQ ID NO: 978 - DNA, 92 bp, Artificial Sequence
  Probe
aggaccggatcaactacaacgttagtttctcgagcaggtgagtagaagatcggaagagcgtcgtgtagggaaagagtcattgcgtgaaccga

SEQ ID NO: 979 - DNA, 20 bp, Artificial Sequence
  primer
tgcctaggaccggatcaact

SEQ ID NO: 980 - DNA, 20 bp, Artificial Sequence
  primer
  modified_base (1)..(1): biotinylated
gagcttcggttcacgcaatg

SEQ ID NO: 981 - DNA, 20 bp, Artificial Sequence
  primer
gagcttcggttcacgcaatg

(19) United States
(12) Patent Application Publication
(10) Pub. No.: US 2021/0164021 A1
(43) Pub. Date: Jun. 3, 2021

(54) NUCLEIC ACID AMPLIFICATION METHOD

(71) Applicant: Keygene N.V., Wageningen (NL)

(72) Inventors: Rene Cornelis Josephus HOGERS, Wageningen (NL); Maria Johanna BLEEKER, Wageningen (NL); Michael Josephus Theresia VAN EIJK, Wageningen (NL)

(73) Assignee: Keygene N.V., Wageningen (NL)

(21) Appl. No.: 17/118,429

(22) Filed: Dec. 10, 2020

Related U.S. Application Data
(63) Continuation of application No. PCT/EP2019/065367, filed on Jun. 12, 2019.

(30) Foreign Application Priority Data
Jun. 12, 2018 (EP) 18177178.3

(51) Int. Cl. C12Q 1/6806; C12Q 1/6834

(52) U.S. Cl. CPC C12Q 1/6806; C12Q 1/6834

(57) ABSTRACT

The invention concerns a method for the production of oligonucleotides. The method of the invention uses a combination of amplification, restriction and affinity purification to produce high quality oligonucleotides. The invention further pertains to a nucleic acid precursor for use in the method of the invention, a solid support comprising said nucleic acid precursor and a kit for use in the method of the invention.

### CROSS-REFERENCE TO RELATED APPLICATIONS

This application is a continuation of International Application No. PCT/EP2019/065367 filed Jun. 12, 2019, and claims the benefit of priority to European Patent Application No. 18177178.3, filed Jun. 12, 2018, the entire contents of both of which are incorporated herein by reference in their entireties.

The instant application contains a Sequence Listing which has been submitted in ASCII format via EFS-WEB and is hereby incorporated by reference in its entirety. Said ASCII copy, created on Nov. 10, 2020, is 247 KB and is named 085342-2050_SequenceListing.txt.

### FIELD OF THE INVENTION

The present invention relates to the field of molecular biology and biotechnology. In particular the invention relates to the production of oligonucleotides, more in particular targeting oligonucleotides or nucleic acid probes that are suitable for use, amongst others, in the field of nucleic acid detection, such as (high throughput) detection of nucleic acids, targeted variation detection and targeted and/or programmable genome editing.

The present invention is in particular useful in the field of high throughput detection of nucleic acids and/or nucleic acid variations.

### BACKGROUND ART

With the near exponential increment of genetic information becoming available due to the development of advanced technologies for obtaining information on traits, alleles and sequencing, there is a growing need for efficient, reliable, scalable assays to test samples and in many cases multiple samples in a rapid, often parallel fashion. In particular single nucleotide polymorphisms (SNPs) contain valuable information on the genetic make-up of organisms and the detection thereof is a field that has attracted a lot of interest and innovative activity.

One of the principal methods used for the analysis of the nucleic acids of a known sequence is based on annealing two probes to a target sequence and, when the probes are hybridised adjacently to the target sequence, ligating the probes. Detection of a successful ligation event is then indicative for the presence of the target sequence in the sample. The Oligonucleotide Ligation Assay (OLA) is a technology that has been found suitable for the detection of such single nucleotide polymorphisms and has over the years been described in many variations in a number of patent applications and scientific articles.

The OLA-principle (Oligonucleotide Ligation Assay) has been described, amongst others, in U.S. Pat. No. 4,988,617 (Landegren et al.). This publication discloses a method for determining the nucleic acid sequence in a region of a known nucleic acid sequence having a known possible mutation or polymorphism. To detect the mutation, oligonucleotides are selected to anneal to immediately adjacent segments of the sequence to be determined. One of the selected oligonucleotide probes has an end region wherein one of the end region nucleotides is complementary to either the normal or to the mutated nucleotide at the corresponding position in the known nucleic acid sequence. A ligase is provided which covalently connects the two probes when they are correctly base paired and are located immediately adjacent to each other. The presence, absence or amount of the linked probes is an indication of the presence of the known sequence and/or mutation. Other variants of OLA-based techniques have been disclosed inter alia in Nilsson et al. Human mutation, 2002, 19, 410-415; Science 1994, 265: 2085-2088; U.S. Pat. No. 5,876,924; WO98/04745; WO98/04746; US6,221,603; U.S. Pat. Nos. 5,521,065; 5,962,223; EP185494131; U.S. Pat. Nos. 6,027,889; 4,988,617; EP246864B1; U.S. Pat. No. 6,156,178; EP745140 B1; EP964704 B1; WO03/054511; US2003/0119004; US2003/190646; EP1313880; US2003/0032016; EP912761; EP956359; US2003/108913; EP1255871; EP1194770; EP1252334; WO96/15271; WO97/45559; US2003/0119004A1; U.S. Pat. No. 5,470,705; WO01/57269; WO03/006677; WO01/061033; WO2004/076692; WO2006/076017; WO2012/019187; WO2012/021749; WO2013/106807; WO2015/154028; WO2015/014962 and WO2013/009175.

Further advancements in the OLA techniques have been reported by KeyGene, Wageningen, the Netherlands. In WO2004/111271, WO2005/021794, WO2005/118847 and WO03/052142, they have described several methods and probe designs that improved the reliability of oligonucleotide ligation assays. These applications further disclose the significant improvement in multiplex levels that can be achieved. Also “SNPWave: a flexible multiplexed SNP genotyping technology”, van Eijk M J, et al., Nucleic Acids Res. 2004; 32(4):e47) describes the improvements made in this field.

With the onset of Next Generation Sequencing (NGS) technologies such as described in Janitz Ed. Next Generation Genome sequencing, Wiley VCH, 2008 and available on the market in platforms provided for by Roche (GS FLX and related systems) and Illumina (Genome Analyzer and related systems), the need arose to adapt the OLA assay to sequencing as a detection platform. Improvements in that field have been described inter alia in WO 2007100243 of Keygene N V. In WO2007100243, the application of next generation sequencing technology to the results of oligonucleotide ligation assays have been described. There remains a need for further improvements in this field, not only from the point of reliability and accuracy, but also from economic drivers, to further reduce the costs by increasing scale.

For example, there is a continuing need for the economic production of high quality oligonucleotide probes. Such high quality oligonucleotides are suitable for use, amongst others, in multiplex reactions such as multiplex OLA assays as described herein above. OLA assays typically require three specific probes to specify each target. At high degrees of multiplexing, the number and amount of oligonucleotides required is potentially very expensive as they are typically synthesized and purified individually. Porreca already addressed this problem in 2007 (Porreca et al. Multiplex amplification of large sets of human exons, Nature Methods-4, 931-936 (2007)) and disclosed a method for amplification of multiple oligonucleotide probes (100-mers) synthesized in parallel on a solid surface for use in a method for targeted amplification of nucleic acids. Porreca et al. described a method using PCR amplification of probes each comprising a 70 nt contiguous protein coding sequence in the human genome flanked by sequences containing recognition sites for nicking restriction endonucleases at their junction with the targeting arms. The amplicons were digested using REs, column-purified, separated on acrylamide gel, recovered from a band corresponding to the expected single-stranded 70 nt species and purified. According to the paper, this process results in the amplification of 2.5 nM oligonucleotides in 200 μL, i.e. an amount of 0.5 pMol, to 125 nM oligonucleotides in 20 μL, i.e. an amount of 2.5 pMol. In other words, a 5 fold amplification was reported.

The present inventors have reworked the method of Porreca for probe amplification, and found similar results when using a relative high amount of input material (0.5 pmol) of nine probe precursors with an average length of 90 nt (85-93 nt), i.e. an amplification factor of 4.5. Such yield is not satisfactory for use of high-throughput targeted nucleotide detection such as OLA. Further, although a 3-plex assay (suitable for SNP detection in 3 different target sequences and requiring 9 different probe sequences) resulted in relatively clean amplification products, increasing the number of probes to a 326-plex assay (978 different probe sequences) resulted in background bands which is likely due to hetero-duplex formation that may hamper the yield and sequence composition due to PCR amplification artifacts.

Hence, there is still a need in the art for a method to increase the molar amount and/or yield of pooled oligonucleotides, e.g. synthesized in low quantities on arrays, without changing their sequence composition and perturbing the relative abundance of each oligo in the pool significantly. There is a need for the production of these oligonucleotides at a sufficient quantity and quality to allow development of highly multiplexed assays for high-throughput analysis of thousands of samples.

The inventors now found an improved oligonucleotide amplification method resulting in high yield, i.e. after purification resulting in a 500-fold amplification factor even for 326-plex assays suitable for high throughput detection methods. The invention is set out in further detail throughout the description, the figures and the various embodiments described herein. All references cited are incorporated herein.

### SUMMARY OF THE INVENTION

In a first aspect, the invention pertains to a method for producing one or more single-stranded oligonucleotides having a sequence of interest, wherein the method comprises the steps of:
a) providing at least one single- or double-stranded nucleic acid precursor comprising a first strand and optionally a second strand that is complementary to the first strand, wherein the first strand comprises the following elements in a 5′ to 3′ direction:
   (1) the first primer binding site;
   (2) a first endonuclease recognition site;
   (3) the sequence of interest;
   (4) a second endonuclease recognition site; and,
   (5) a second primer binding site;
wherein the first endonuclease recognition site is designed such that, after duplexing, a first endonuclease cleaves the sugar-phosphate backbone of the first strand immediately upstream of the sequence of interest; and, wherein the second endonuclease recognition site is designed such that, after duplexing, a second endonuclease cleaves the sugar-phosphate backbone of the first strand immediately downstream of the sequence of interest;
b) amplifying the precursor of step a) by an amplification method, using a first primer capable of hybridizing to the first primer binding site and a second primer capable of hybridizing to the second primer binding site;
c) digesting the amplified double-stranded precursor obtained in step b) with the first and the second endonuclease to produce an amplified double-stranded nucleic acid precursor with cleavages of the sugar-phosphate backbone immediately up- and downstream of the sequence of interest; and
e) denaturing the amplified double-stranded precursor, thereby releasing the single-stranded oligonucleotide having the sequence of interest.

Preferably, the first primer can selectively anneal to only the first primer binding site (more specifically, to the primer binding sequence comprised within the first primer binding site of the second strand) and the second primer can selectively anneal to only the second primer binding site (more specifically, to the primer binding sequence comprised within the second primer binding site of the first strand). Optionally the first and second primer may be identical, or similar in the sense that the first primer can anneal to both the first and the second primer binding site and the second primer can anneal to both the first and the second primer binding site. Optionally, this primer can selectively anneal to only the first and second primer binding site.

Preferably, the sequence of interest does not comprise the first and/or the second endonuclease recognition site or reverse complement thereof.

In a preferred embodiment, the method of the invention further comprises one or more steps in order to separate the oligonucleotide comprising the sequence that is complementary to the sequence of interest from the first strand, or from the remainder of the first strand comprising the sequence of interest. Preferably, this is accomplished by adding a step d) of immobilizing the second strand, or remainder of the second strand comprising at least the sequence complementary to the sequence of interest:
i) between amplification step b) and digestion step c),
ii) between digestion step c) and the denaturing step e); or,
iii) after the denaturing step e).

Preferably, this immobilizing step involves affinity capturing the second strand, or part thereof comprising the sequence that is complementary to the sequence of interest, on a solid support. This may require tagging of the second strand as a whole, or the part thereof comprising the sequence that is complementary to the sequence of interest. Tagging of the second strand as a whole may be achieved using a second primer in step b) of the method of the invention comprising an affinity tag. The affinity tag can be present on at least the second primer. It is further understood herein that the affinity tag can be present on both the first primer and second primer. Alternatively, the affinity tag is only present on the second primer, i.e. it is not present on the first primer. The first and second primer are used to produce an amplified double-stranded nucleic acid precursor comprising the tag. Alternatively, the second primer used in step b) may be present on a solid support prior to amplification, wherein amplification in step b) is performed on a solid support resulting in amplicons attached to the solid support via the second strand. A further step of removing the second strand, or part thereof comprising the reverse complement of the sequence of interest, is added to the method of the invention to obtain a single-stranded oligonucleotide having the sequence of interest. Said removal step is preferably added after the denaturing step in option i) or ii) as defined above, or after the immobilization step in option iii) as defined above. Preferably, within this embodiment, the precursor or method is designed such that digesting the amplified double-stranded precursor as defined in step c) of the method of the invention maintains the sugar-phosphate backbone of the second strand between the tag up to and including the sequence of interest intact.

Preferably, the method of the invention further comprises a step g) of purifying the single-stranded oligonucleotide.

In a preferred embodiment, the denaturing in step e) comprises chemical denaturing, wherein preferably the chemical denaturing is by increasing the pH by the addition of a strong base, preferably by the addition of an alkali hydroxide at a concentration of about 0.5-1.5 M.

Preferably, the nucleic acid precursor consists of about 20-200 nucleotides, and wherein preferably the nucleic acid precursor has a sequence selected from the group consisting of SEQ ID NO: 1-SEQ ID NO: 978.

Preferably, the sequence of interest is at least partly complementary to a predetermined genomic sequence, wherein preferably the produced oligonucleotide is suitable for use in a multiplex OLA assay and wherein more preferably the produced oligonucleotide is suitable for use in an at least 300-plex OLA assay.

Preferably, the nucleic acid precursor is a single-stranded nucleic acid precursor. In a preferred embodiment, the amplification method in step b) is an isothermal amplification method, wherein preferably the isothermal amplification method is Recombinase Polymerase Amplification (RPA) or Helicase Dependent Amplification (HDA).

Preferably, the first and the second endonuclease in step c) are two different enzymes.

Preferably, the first endonuclease in step c) cleaves: i) the first DNA strand; or ii) the first and the second DNA strand.

In a preferred embodiment, the amplified double-stranded precursor from step b) is purified prior to binding the solid support in step d).

Preferably, the tag for affinity capturing the second strand, or part thereof, is biotin and the solid support comprises streptavidin, wherein preferably the solid support is a bead and wherein more preferably the bead is a magnetic bead.

Preferably, in step a) two or more nucleic acid precursors are provided that have a distinct sequence of interest, wherein preferably the sequences of the nucleic acid precursors are selected from the group consisting of SEQ ID NO: 1-SEQ ID NO: 978.

In a second aspect, the invention concerns a single- or a double-stranded nucleic acid precursor comprising a first strand and optionally a second strand that is complementary to the first strand, wherein the first strand comprises the following elements in a 5′ to 3′ direction:
(1) a first primer binding site;
(2) a first endonuclease recognition site;
(3) the sequence of interest;
(4) a second endonuclease recognition site; and,
(5) a second primer binding site;
wherein a first primer can selectively anneal to only the first primer binding site and a second primer can selectively anneal to only the second primer binding site;
wherein the sequence of interest does not comprise the first and the second endonuclease recognition sites or reverse complement thereof;
wherein the first endonuclease recognition site is designed such that, after duplexing, a first endonuclease cleaves the sugar-phosphate backbone of the first strand immediately upstream of the sequence of interest; and,
wherein the second endonuclease recognition site is designed such that, after duplexing, a second endonuclease cleaves the sugar-phosphate backbone of the first strand immediately downstream of the sequence of interest.
   Preferably, the precursor further comprises an affinity tag located at the 5′ end of the second strand, preferably the affinity tag is not located at the 5′ end of the first strand, preferably the affinity tag is only located at the 5′ end of the second strand.

In a third aspect, the invention concerns the double-stranded nucleic acid precursor as defined herein bound to the solid support by means of affinity-capture.

In a fourth aspect, the invention pertains to a kit of parts for use in a method of the invention comprising:
a container comprising the second endonuclease and optionally the first endonuclease;
a container comprising enzymes for use in amplification step b) of the method of the first aspect, optionally in combination with the first and/or tagged second primer;
a container comprising a solid support for affinity purification; and optionally
a container comprising a chemical for denaturation.

In a fifth aspect, the invention concerns the use of a nucleic acid precursor as defined herein or a kit of parts as defined herein for the production of one or more single-stranded oligonucleotides.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

The singular terms “a,” “an,” and “the” include plural referents unless the context clearly dictates otherwise. Thus, for example, reference to “a cell” includes a combination of two or more cells, and the like.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ±(+ or −) 10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

“Construct” or “nucleic acid construct” or “vector”: this refers to a man-made nucleic acid molecule resulting from the use of recombinant DNA technology and which is used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g. a coding sequence) is integrated downstream of the transcription regulatory sequence. Vectors may comprise further genetic elements to facilitate their use in molecular cloning, such as e.g. selectable markers, multiple cloning sites and the like.

“Sequence” or “Nucleotide sequence”: This refers to the order of nucleotides of, or within a nucleic acid. In other words, any order of nucleotides in a nucleic acid may be referred to as a sequence or nucleotide sequence.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide.

The term “complementarity” is herein defined as the sequence identity of a sequence to a fully complementary strand (defined herein below, e.g. the second strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWlN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3rd edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

Likewise, a “target sequence” is to denote an order of nucleotides within a nucleic acid that is to be targeted, e.g. wherein an alteration is to be introduced or to be detected. For example, the target sequence is an order of nucleotides comprised by a first strand of a DNA duplex.

An “endonuclease” is an enzyme that hydrolyses at least one strand of a duplex DNA upon binding to its recognition site. An endonuclease is to be understood herein as a site-specific endonuclease. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

A primer binding site is herein defined as a site that upon duplexing comprises a primer binding sequence to which a primer sequence can selectively hybridize. A primer binding sequence is hence preferably a single-stranded DNA sequence.

An endonuclease recognition site is defined herein as comprising a specific sequence to which, when duplexed, an endonuclease can bind and subsequently hydrolyse at least one strand of DNA. The specific sequence that is recognized by the endonuclease may be located in the first strand or in the second strand of the duplex DNA. The double-stranded or single-stranded break that is generated by the endonuclease may be located within the endonuclease recognition site. Preferably, the break may be located directly adjacent to the endonuclease recognition sequence, or one or more (e.g. 1, 2, 3, 4, 5, 6, 7, 8, 9, 10, 11, 12, 13, 14, 15 or 16) bases upstream of downstream of the endonuclease recognition sequence.

### DETAILED DESCRIPTION OF THE INVENTION

In a first aspect, the invention pertains to a method for producing one or more single-stranded oligonucleotides having a sequence of interest, wherein the method comprises the steps of:
a) providing at least one single- or double-stranded nucleic acid precursor comprising a first strand and optionally a second strand that is complementary to the first strand, wherein the first strand comprises the following elements in a 5′ to 3′ direction:
   (1) a first primer binding site;
   (2) a first endonuclease recognition site;
   (3) the sequence of interest;
   (4) a second endonuclease recognition site; and,
   (5) a second primer binding site;
wherein the first endonuclease recognition site is designed such that, after duplexing, a first endonuclease cleaves the sugar-phosphate backbone of the first strand immediately upstream of the sequence of interest; and, wherein the second endonuclease recognition site is designed such that, after duplexing, a second endonuclease cleaves the sugar-phosphate backbone of the first strand immediately downstream of the sequence of interest;
b) amplifying the precursor of step a) by an amplification method, using a first primer capable of hybridizing to the first primer binding site and a second primer capable of hybridizing to the second primer binding site;
c) digesting the amplified double-stranded precursor obtained in step b) with the first and the second endonuclease to produce an amplified double-stranded nucleic acid precursor with cleavages of the sugar-phosphate backbone immediately up- and downstream of the sequence of interest; and
e) denaturing the amplified double-stranded precursor, thereby releasing the single-stranded oligonucleotide having the sequence of interest.

Additional steps may be included in the method of the invention, such as an additional purifying step or (long term or short term) storage of the obtained product or any other suitable additional method step.

The first strand comprises the sequence of interest. Hence, the first strand is to be understood herein as the strand of the nucleic acid precursor or of the nucleic acid amplified therefrom by step b) of the method of the invention, comprising the sequence of interest. The second strand comprises the sequence complementary to the sequence of interest. The second strand is to be understood herein as the strand of the nucleic acid precursor or of the nucleic acid amplified therefrom by step b) of the method of the invention, complementary to the first strand.

It is to be understood herein, the first primer binding site of the first strand comprises the reverse complement of a first primer binding sequence, such that the complement strand (also indicated herein as the second strand) will comprise a first primer binding sequence within this first primer binding site to which the first primer can selectively anneal. It is further to be understood herein, that the second primer binding site of the first strand comprises a second primer binding sequence in the first strand to which the second primer can selectively anneal. Preferably, the first primer can selectively anneal only the first primer binding sequence and the second primer can selectively anneal to only the second primer binding sequence. Optionally the first and second primer may be identical, or similar in the sense that the first primer can anneal to both the first and the second primer binding sequence and the second primer can anneal to both the first and the second primer binding sequence. Optionally, the (first and second) primer can selectively anneal to only both the first and second primer binding site.

Preferably, the sequence of interest does not comprise the first and/or the second endonuclease recognition site or reverse complement thereof.

In a preferred embodiment, the method of the invention further comprises one or more steps in order to separate the oligonucleotide comprising the sequence that is complementary to the sequence of interest from the first strand, or from the remainder of the first strand comprising the sequence of interest. Preferably, this is accomplished by adding a step d) of immobilizing the second strand, or remainder second strand comprising the sequence complementary to the sequence of interest:
i) between amplification step b) and digestion step c),
ii) between digestion step c) and the denaturing step e); or,
iii) after the denaturing step e).

Preferably, this immobilizing step involves affinity capturing the second strand, or part thereof comprising the sequence that is complementary to the sequence of interest, on a solid support. This may require tagging of the second strand as a whole, or part thereof comprising the sequence that is complementary to the sequence of interest. Tagging of the second strand as a whole may be achieved using a second primer in step b) of the method of the invention comprising an affinity tag, to produce an amplified double-stranded nucleic acid precursor comprising the tag.

The affinity tag can be present on at least the second primer. It is further understood herein that an affinity tag can be present on both the first primer and second primer. Alternatively, the affinity tag is not present on the first primer, e.g. the affinity tag is only present on the second primer.

In another embodiment, the second primer used in step b) can be present on a solid support as specified herein prior to amplification, wherein amplification in step b) is performed on a solid support resulting in amplicons attached to the solid support via the second strand. Within this embodiment, the first primer for amplification can be provided separately from the solid support, e.g. can be present in solution, and the second primer may be linked to the solid support, for example by covalent linkage or immobilized via affinity capturing as further detailed herein.

A further step of removing the second strand, or part thereof comprising the reverse complement of the sequence of interest, is added to the method of the invention to obtain a single-stranded oligonucleotide having the sequence of interest. Said removal step is preferably added after the denaturing step in option i) or ii) as defined above, or after the immobilization step in option iii) as defined above. Preferably, within this embodiment, the precursor or method is designed such that digesting the amplified double-stranded precursor as defined in step c) of the method of the invention maintains the sugar-phosphate backbone of the second strand between the tag up to and including the sequence of interest intact.

Therefore, a preferred embodiment of the method of the invention comprises the steps of:
a) providing at least one single- or double-stranded nucleic acid precursor comprising a first strand and optionally a second strand that is complementary to the first strand, wherein the first strand comprises the following elements in a 5′ to 3′ direction:
   (1) a first primer binding site;
   (2) a first endonuclease recognition site;
   (3) the sequence of interest;
   (4) a second endonuclease recognition site; and,
   (5) a second primer binding site;
wherein a first primer can selectively anneal to only the first primer binding site and a second primer can selectively anneal to only the second primer binding site;
wherein the sequence of interest does not comprise the first and the second endonuclease recognition sites or reverse complements thereof,
wherein the first endonuclease recognition site is designed such that, after duplexing, the first endonuclease cleaves the sugar-phosphate backbone of the first strand immediately upstream of the sequence of interest; and,
wherein the second endonuclease recognition site is designed such that, after duplexing, the second endonuclease cleaves the sugar-phosphate backbone of the first strand immediately downstream of the sequence of interest;
b) amplifying the precursor of step a) by an amplification method, using the first primer capable of hybridizing to the first primer binding site and the second primer capable of hybridizing to the second primer binding site, wherein at least the second primer comprises an affinity tag, to produce an amplified double-stranded nucleic acid precursor comprising the tag, Preferably the affinity tag is not present on the first primer;
c) digesting the amplified double-stranded precursor obtained in step b) with the first endonuclease and with the second endonuclease to produce an amplified double-stranded nucleic acid precursor with cleavages of the sugar-phosphate backbone immediately up- and downstream of the sequence of interest and with an intact sugar-phosphate backbone between the tag up to and including the sequence complementary to the sequence of interest;
d) immobilizing the amplified double-stranded nucleic acid precursor on a solid support by affinity capture of the tagged complementary second strand;
e) denaturing the amplified double-stranded precursor, thereby releasing the single-stranded oligonucleotide having the sequence of interest; and
f) removing the solid support to obtain the single stranded oligonucleotide having the sequence of interest.

A schematic representation of a preferred embodiment of the invention is depicted in FIG.

1. The skilled person understands that method of the invention may comprise the steps as detailed above. However, it is not essential for the invention that the steps are performed in the order specified above. In a preferred embodiment, step c) and step d) are reversed. In an alternative embodiment, step d) and step e) are reversed.

Hence in a preferred embodiment of the invention, the method may comprise the steps specified above (and further detailed below) in the following order:
i) step a), step b), step c), step d), step e) and step f); or
ii) step a), step b), step d), step c), step e) and step f); or
iii) step a), step b), step c), step e), step d) and step f).
   Therefore, optionally, the method of the invention may comprises the following subsequent steps:
a) providing at least one single- or double-stranded nucleic acid precursor comprising a first strand and optionally a second strand that is complementary to the first strand, wherein the first strand comprises the following elements in a 5′ to 3′ direction:
   (1) a first primer binding site;
   (2) a first endonuclease recognition site;
   (3) the sequence of interest;
   (4) a second endonuclease recognition site; and,
   (5) a second primer binding site;
wherein a first primer can selectively anneal to only the first primer binding site and a second primer can selectively anneal to only the second primer binding site;
wherein the sequence of interest does not comprise the first and the second endonuclease recognition sites or reverse complements thereof,
wherein the first endonuclease recognition site is designed such that, after duplexing, the first endonuclease cleaves the sugar-phosphate backbone of the first strand immediately upstream of the sequence of interest; and,
wherein the second endonuclease recognition site is designed such that, after duplexing, the second endonuclease cleaves the sugar-phosphate backbone of the first strand immediately downstream of the sequence of interest;
b) amplifying the precursor by an amplification method, using the first primer capable of hybridizing to the first primer binding site and the second primer capable of hybridizing to the second primer binding site, wherein the second primer comprises an affinity tag, to produce an amplified double-stranded nucleic acid precursor comprising the tag, wherein preferably the affinity tag is not present on the first primer;
d) immobilizing the amplified double-stranded nucleic acid precursor on a solid support by affinity capture of the tagged complementary second strand;
c) digesting the amplified double-stranded precursor with the first endonuclease and with the second endonuclease to produce an amplified double-stranded nucleic acid precursor with cleavages of the sugar-phosphate backbone immediately up- and downstream of the sequence of interest and with an intact sugar-phosphate backbone between the tag up to and including the sequence complementary to the sequence of interest;
e) denaturing the amplified double-stranded precursor, thereby releasing the single-stranded oligonucleotide having the sequence of interest; and
f) removing the solid support to obtain the single stranded oligonucleotide having the sequence of interest.
   In addition, the method of the invention may comprises the following subsequent steps:
a) providing at least one single- or double-stranded nucleic acid precursor comprising a first strand and optionally a second strand that is complementary to the first strand, wherein the first strand comprises the following elements in a 5′ to 3′ direction:
   (1) a first primer binding site;
   (2) a first endonuclease recognition site;
   (3) the sequence of interest;
   (4) a second endonuclease recognition site; and,
   (5) a second primer binding site;
wherein a first primer can selectively anneal to only the first primer binding site and a second primer can selectively anneal to only the second primer binding site;
wherein the sequence of interest does not comprise the first and the second endonuclease recognition sites or reverse complements thereof,
wherein the first endonuclease recognition site is designed such that, after duplexing, the first endonuclease cleaves the sugar-phosphate backbone of the first strand immediately upstream of the sequence of interest; and,
wherein the second endonuclease recognition site is designed such that, after duplexing, the second endonuclease cleaves the sugar-phosphate backbone of the first strand immediately downstream of the sequence of interest;
b) amplifying the precursor of by an amplification method, using the first primer capable of hybridizing to the first primer binding site and the second primer capable of hybridizing to the second primer binding site, wherein the second primer comprises an affinity tag, to produce an amplified double-stranded nucleic acid precursor comprising the tag, wherein preferably the affinity tag is not present on the first primer;
c) digesting the amplified double-stranded precursor with the first endonuclease and with the second endonuclease to produce an amplified double-stranded nucleic acid precursor with cleavages of the sugar-phosphate backbone immediately up- and downstream of the sequence of interest and with an intact sugar-phosphate backbone between the tag up to and including the sequence complementary to the sequence of interest;
e) denaturing the amplified double-stranded precursor, thereby releasing the single-stranded oligonucleotide having the sequence of interest;
d) immobilizing the tagged complementary second strand of the denatured amplified double-stranded nucleic acid precursor on a solid support by affinity capture; and
f) removing the solid support to obtain the single stranded oligonucleotide having the sequence of interest.

Additional purification steps or the additional purification step may be included e.g. in between step a) and step b), and/or in between step b) and step c), and/or in between step c) and step d), and/or in between step d) and step e), and/or in between step e) and step f), and/or in between step d) and step c), and/or in between step e) and step d), and/or in between step b) and step d), and/or in between step c) and step e), and/or in between step d) and step f), and/or after step f).

Alternatively, the method can consist of the following steps as defined above
i) step a), step b), step c), step d), step e) and step f); or
ii) step a), step b), step d), step c), step e) and step f); or
iii) step a), step b), step c), step e), step d) and step f).

In case the amplification in step b) is performed on a solid support as detailed above, the method may comprise the steps specified above (and further detailed below) in the following order: step a), step b), step c), step e) and step f). In other words, the method of the invention may comprise the following consecutive steps:
a) providing at least one single- or double-stranded nucleic acid precursor comprising a first strand and optionally a second strand that is complementary to the first strand, wherein the first strand comprises the following elements in a 5′ to 3′ direction:
   (1) a first primer binding site;
   (2) a first endonuclease recognition site;
   (3) the sequence of interest;
   (4) a second endonuclease recognition site; and,
   (5) a second primer binding site;
wherein a first primer can selectively anneal to only the first primer binding site and a second primer can selectively anneal to only the second primer binding site;
wherein the sequence of interest does not comprise the first and the second endonuclease recognition sites or reverse complements thereof,
wherein the first endonuclease recognition site is designed such that, after duplexing, a first endonuclease cleaves the sugar-phosphate backbone of the first strand immediately upstream of the sequence of interest; and,
wherein a second endonuclease recognition site is designed such that, after duplexing, the second endonuclease cleaves the sugar-phosphate backbone of the first strand immediately downstream of the sequence of interest;
b) amplifying the precursor of step a) by an amplification method, using the first primer capable of hybridizing to the first primer binding site and the second primer capable of hybridizing to the second primer binding site, wherein the second primer is linked to a solid support, to produce an amplified double-stranded nucleic acid precursor comprising the tag;
c) digesting the amplified double-stranded precursor obtained in step b) with the first endonuclease and with the second endonuclease to produce an amplified double-stranded nucleic acid precursor with cleavages of the sugar-phosphate backbone immediately up- and downstream of the sequence of interest and with an intact sugar-phosphate backbone between the tag up to and including the sequence complementary to the sequence of interest;
e) denaturing the amplified double-stranded precursor, thereby releasing the single-stranded oligonucleotide having the sequence of interest; and optionally,
f) removing the solid support to obtain the single stranded oligonucleotide having the sequence of interest.

One or more additional purification steps may be included e.g. in between step a) and step b), and/or in between step b) and step c), and/or in between step c) and step e), and/or in between step e) and step f), and/or after step f). Alternatively, within this embodiment wherein amplification is applied on a solid support, the method may consist of the following steps as defined above in this embodiment: step a), step b), step c), step e) and step f). As the sequence of interest is already comprised within the nucleic acid precursors provided in step a) of the method of the invention, the method of the invention may also be considered a method of amplification of one or more single-stranded oligonucleotides having a sequence of interest.

The invention is described in more detail below:

### Oligonucleotide Having a Sequence of Interest

In the first aspect, the invention pertains to a method for producing one or more single-stranded oligonucleotides having a sequence of interest. A single-stranded oligonucleotide is defined herein as a short single-stranded DNA or RNA molecule. In a preferred embodiment, the single-stranded oligonucleotide is a single-stranded DNA molecule. The method is in particular suitable for the pooled production (i.e. the production in a single vessel) of high numbers of oligonucleotides with optionally different sequences, e.g. different sequences of interest, using an initial pool of multiple precursor oligonucleotides comprising these optionally different sequences, as defined under “Nucleic acid precursor” herein further, as starting material in step a) of the method of the invention.

In a preferred embodiment, the produced single-stranded oligonucleotide, or the pool of single stranded oligonucleotides, consists of, or each consist of, about 20-200 nucleotides, preferably of about 30-180 nucleotides, about 40-160 nucleotides, about 50-140 nucleotides, about 60-120 nucleotides, about 70-110 nucleotides, about 75-100 nucleotides, about 75-95 nucleotides or about 80-90 nucleotides. It is to be understood that these nucleotides are preferably contiguous nucleotides.

Preferably, the produced oligonucleotide, or the pool of single stranded oligonucleotides, consists of, or each consist of, at least about, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 nucleotides and/or does not have more than 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25 or 20 nucleotides.

In an exemplified embodiment of the invention further detailed herein, the nucleic acid precursor has a sequence selected from the group consisting of SEQ ID NO: 1-SEQ ID NO: 978, preferably the sequence is selected from the group consisting of SEQ ID NO: 1-326, the group consisting of SEQ ID NO: 327-652 and/or the group consisting of SEQ ID NO: 653-978. Most preferably, the nucleic acid precursor has a sequence selected from the group consisting of SEQ ID NO: 653-978. The pool of nucleic acid precursors used as starting material in this embodiment comprises or consists of a pool of these 978 nucleic acid precursors represented by SEQ ID NO: 1-SEQ ID NO: 978.

The single-stranded oligonucleotide to be produced, or the pool of single-stranded oligonucleotides, may comprise or consist of, or may each comprise or consist of, a sequence of interest. Preferably, the single-stranded oligonucleotide, or the pool of single stranded oligonucleotides, produced by the method of the invention consists of, or each consist of, the sequence of interest. Particularly preferred sequences of interest are sequences that can be used e.g. as a primer for amplification, as a probe for ligation, hybridization or (in solution) capturing or as adaptor or as a template for in vitro transcription.

A sequence of interest for use as a primer, or primer oligonucleotide, may comprise a sequence that is at least in part complementary to a predetermined target sequence to be amplified, such as a predetermined (genomic) DNA sequence, cDNA sequence, RNA sequence and/or cell free DNA sequence. Such sequence is denominated herein as a complementary target sequence. Preferably, said complementary target sequence is at least 80%, 85%, 90%, 98% or 99% complementary to a predetermined target sequence. Most preferably, the complementary target sequence is fully complementary (100%) to a predetermined target sequence. Preferably, such complementary target sequence is a stretch of about 18, 19, 20, 21, 22, 23, nucleotides in length. Optionally, the sequence of interest for use as primer comprises further functional elements, such as one or more primer binding sites for subsequent amplification and/or sequencing step(s), and/or one or more barcoding sequences (optionally interrupted barcodes such as described in WO2016/201142), e.g. for sample tracing or molecular indexing, and/or one or more degenerate nucleotides. The primer may be a tailed primer, which is understood herein as a primer comprising a complementary target sequence at the 3′ end and a tail comprising one or more functional elements, preferably the functional elements as indicated above. Alternatively, the primer may be an omega primer such as described in US 2008/0305478 A1, US 2010/0227320 A1, US 2016/0068903 A1. Such omega primer typically comprises two complementary target sequences at both the 3′ and 5′ end of the primer (typically a stretch of 6-60 nucleotides in length and a stretch of 10-100 nucleotides in length, respectively) separated by a loop (typically a stretch of 12-50 nucleotides in length) which does not bind to the target and which may subsequently be used as a priming section for monoplex PCR.

The method of the invention is in particular suitable for the production of a defined pool of primer oligonucleotides that can be used for instance in multiplex oligonucleotide-based amplification such as multiplex PCR. Such primer pool may comprise or consist of primer pairs, which together are suitable for amplifying a particular target sequence. Optionally, both primers of the pair are target specific, which is to be understood herein as that at least part of the primer comprises as sequence that is complementary to a specific sequence to be amplified, which may be a certain gene or part thereof. Alternatively, one primer of the pair is a so called common primer, which may be capable of annealing to a sequence that is not specific to a particular target sequence, e.g. a pre-determined sequence in an adapter while the other primer of the pair is target specific. Optionally, both primers of the pair are common primers. In case the primers of the pair are tailed primers, the tail may comprise universal sequences for subsequent tail PCR with a pair of common primers.

The produced oligonucleotide is suitable for use as a primer in an at least 10-, 20, 40-, 60-, 80-, 100-, 120-, 140-, 160-, 180-, 200-, 220-, 240-, 260-, 280-, 300-, 320-, 326-, 340-, 360, 380-, 400-, 420-, 440-, 460-, 480-, 500-, 600-, 700-, 800-, 900-, 1,000-, 2,000-, 3,000-, 4,000-, 5,000-, 6,000-, 7,000-, 8,000-, 9,000-, 10,000-, 20,000-, 30,000-, 40,000-, 50,000-, 60,000-, 70,000, 80,000-, 90,000-, 100,000-, 200,000-, 300,000-, 400,000-, or 500,000-plex PCR assay. An n-plex PCR assay is to be understood herein as PCR reactions in a single reaction vessel, resulting in the amplification of n different target sequences. Primers produced by the method of the invention may also be used for sequencing by synthesis or for cloning.

The oligonucleotides produced in a method of the invention are also particularly suitable for use as a probe. Hence, the sequence of interest may consist or comprise a probe sequence. A probe or probe oligonucleotide is herein understood as an oligonucleotide that is used (alone or in combination with one or more other probes) to detect the presence of a nucleotide sequence that is complementary to the sequence in the probe, i.e. a target sequence. Such probe sequences therefore comprises a complementary target sequence as defined above and may further comprise one or more primer binding sites and/or one or more barcoding sequences. A probe may further comprise a tag (label), e.g. an affinity ligand, or a radioactive or fluorescent tag. Oligonucleotide probes produced by the method of the invention are particularly suitable, amongst others, for use in the field of nucleic acid detection, such as (high throughput) detection of nucleic acids by hybridization or (in solution) capturing of nucleic acids (hybridization capture probes), targeted variation detection and targeted and/or programmable genome editing. The method of the invention is in particular suitable for the production of a defined pool of probe oligonucleotides that can be used for instance in multiplex OLA.

A probe may be an OLA probe that, together with another probe can be used for instance in SNP or indel detection. As described in e.g. WO2007/100243, the two target sequences for hybridization of the first and second probe are localized adjacent to each other such that the probes can be ligated directly upon binding, or these two target sequences are not adjacent but leave a gap in between, such that gap filing (Akhunov et al. Theor. Appl. Genet. 2009 August; 119(3):507-517) or gap ligation (using a suitable third oligonucleotide as described e.g. in WO00/77260) is required. In addition, a probe as produced by the method of the invention may also be a padlock probe (e.g., as described in Nilsson et al. Science 1994 Sep. 30; 265(5181): 2085-2088), a molecular inversion probe (e.g., as described in Hardenbol et al. Nat Biotechnol. 2003 June; 21(6):673-678), or a connector inversion probe (e.g., such as described in Akhras et al. PLoS One. 2007; 2(9): e915), which are all single stranded nucleic acid molecules comprising in general two segments (each in general about 20 nucleotides long) complementary to the target and these sections are connected by a linker (e.g., a 40 nucleotides long linker). The nucleic acid molecule becomes circularized upon hybridization to the target sequence and ligation (optionally after gap-filing). The presence of functional in the linker sequence may allow for amplification and subsequent detection.

A particularly preferred predetermined target sequence to be amplified using one or more primers as defined herein and/or detected using one or more probes as defined herein, is a genomic sequence that has a genetic variation, e.g. a nucleotide sequence that contains, represents or is associated with a polymorphism, i.e. a polymorphic site. The term polymorphism herein refers to the occurrence of two or more genetically determined alternative sequences or alleles in a population. In case of a probe, the complementary target sequence is preferably (at least partly) complementary to only one of these two or more genetically determined alternative sequences of the polymorphic site. In case of a primer, the complementary target sequence is preferably (at least partly) complementary to a genetically determined sequence flanking (e.g. upstream or downstream) such polymorphic site.

The polymorphic site may be as small as one base pair, such as a SNP. Polymorphisms include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. In case of a probe, the complementary target sequence is (at least partly) complementary to only one of the two or more genetically determined alternative SNP allele sequences. More preferably, in case of a ligation probe, the nucleotide at the 5′ or 3′ end of the complementary target sequence is complementary to only one of the alternative (SNP) alleles.

In a preferred embodiment, the produced oligonucleotide is suitable for use in an OLA assay. The method of the invention results in the production of high quality single-stranded oligonucleotides. Such oligonucleotides are particularly useful in multiplexing assays, such as, but not limited to multiplex oligonucleotide-based amplification (such as multiplex PCR), multiplex capture hybridization, MLPA and multiplex OLA assays. Preferably, the produced oligonucleotide is suitable for use in an OLA multiplex assay as e.g. described in U.S. Pat. No. 4,988,617; Nilsson et al. (supra); U.S. Pat. No. 5,876,924, WO98/04745; WO98/04746; U.S. Pat. Nos. 6,221,603; 5,521,065; 5,962,223; EP1854941BI; U.S. Pat. Nos. 6,027,889; 4,988,617; EP246864B1; U.S. Pat. No. 6,156,178; EP745140 B1; EP964704 B1; WO03/054511; US2003/0119004; US2003/190646; EP1313880; US2003/0032016; EP912761; EP956359; US2003/108913; EP1255871; EP1194770; EP1252334; WO96/15271; WO97/45559; US2003/0119004A1; U.S. Pat. No. 5,470,705; WO 2004/111271; WO2005/021794; WO2005/118847; WO03/052142; van Eijk M J (supra); WO2007/100243; WO01/57269; WO03/006677; WO01/061033; WO2004/076692; WO2006/076017; WO2012/019187; WO2012/021749; WO2013/106807; WO2015/154028; WO2015/014962 and WO2013/009175.

In a further preferred embodiment, the produced oligonucleotide is suitable for use as a probe in an at least 10-, 20-, 40-, 60-, 80-, 100-, 120-, 140-, 160-, 180-, 200-, 220-, 240-, 260-, 280, 300-, 320-, 326-, 340-, 360-, 380-, 400-, 420-, 440-, 460-, 480-, 500-, 600-, 700-, 800-, 900-, 1,000, 2,000-, 3,000-, 4,000-, 5,000-, 6,000-, 7,000-, 8,000-, 9,000-, 10,000-, 20,000-, 30,000-, 40,000-, 50,000-, 60,000-, 70,000-, 80,000-, 90,000-, 100,000-, 200,000-, 300,000-, 400,000-, or 500,000-plex OLA assay. Preferably the produced oligo is suitable for use in an at least a 300-plex OLA assay, and even more preferably in an at least 326-plex OLA assay.

The oligonucleotide produced by the method of the invention may also be used as a single stranded adapter or for the preparation of partly, or completely, double stranded adapters (such as, but not limited to Y-shape adapters). Partly, or completely, double stranded adapters may be formed by annealing two partly, or completely, complementary single stranded oligonucleotides. Oligonucleotides for use as adapters preferably comprise functional elements, such as but not limited to one or more primer binding sites for amplification step(s) and/or sequencing, and/or one or more barcoding sequences (optionally interrupted barcodes such as described in WO2016/201142), e.g. for sample tracing or molecular indexing, and/or one or more degenerate nucleotides.

### Nucleic Acid Precursor

A first step of the method of the invention is the provision of at least one single- or double-stranded nucleic acid precursor comprising a first strand and optionally a second strand that is complementary to the first strand. The nucleic acid precursor is preferably a DNA molecule.

Hence, the nucleic acid precursor for use in the method of the invention may be a single-stranded nucleic acid precursor comprising a first strand. Alternatively, the nucleic acid precursor for use in the invention may be a double-stranded nucleic acid precursor comprising a first strand and a second strand that is complementary to the first strand. The optional second strand of the nucleic acid precursor is preferably at least 80%, 85%, 90%, 98% or 99% complementary to the first strand. Most preferably, the optional second strand is fully complementary (100%) to the first strand over its entire length.

Preferably, the nucleic acid precursor is a single-stranded nucleic acid precursor and most preferably, the nucleic acid precursor is a single stranded DNA nucleic acid precursor.

The length of the nucleic acid precursor is at least about 50, 60, 70, 80 or 90 nucleotides and preferably a length of at most about 500, 450, 400, 350 or 300 nucleotides, such as between 50 and 500, 50 and 400, 50 and 350, 50 and 300, 80 and 500, 80 and 400, 80 and 350, 80 and 300 nucleotides.

The first strand preferably comprises or consists of the following five elements in a 5′ to 3′ direction:
(1) the first primer binding site;
(2) the first endonuclease recognition site;
(3) the sequence of interest;
(4) the second endonuclease recognition site; and,
(5) the second primer binding site.

These five elements may be five distinct elements (as exemplified in FIG. 2B) or one or more elements may partly or fully overlap (FIG. 2A). For example, the first endonuclease recognition site may be partly or fully comprised within the reverse complement sequence of the first primer binding sequence and/or the second endonuclease recognition site may be partly of fully comprised within the second primer binding sequence. Thus, the same sequence may function as a primer binding sequence as well as an endonuclease recognition site (FIG. 2A).

Hence, the first strand comprises a first primer binding site (having the reverse complement sequence of the first primer binding sequence; upon duplexing the complementary strand will comprise the first primer binding sequence to which the first primer can anneal) and a second primer binding site (having the second primer binding sequence to which the second primer can anneal). Upon duplexing of the first strand (to obtain a first strand and a complementary second strand), a first primer may selectively anneal (e.g. hybridize) to only the first primer binding site and a second primer may selectively anneal (e.g. hybridize) to only the second primer binding site. Put differently, the first primer will not anneal to the nucleic acid precursor and/or its complement, with the exception of the first primer binding site. Similarly, the second primer will not anneal to the nucleic acid precursor or its complement, with the exception of the second primer binding site. Optionally, the first and second primer may be the same or similar in the sense that they anneal to both the first and second primer binding site. In addition, the sequence of the first and second primer binding site may be the same. In other words, the first primer binding sequence may be identical to the second primer binding sequence.

The nucleic acid precursor comprises a sequence of interest as defined above. In a further preferred embodiment, a pool of two or more nucleic acid precursors are provided. Preferably, the pool comprises at least 2, 3, 4, 5, 10, 50, 100, 150, 200, 250, 300, 350, 400, 450, 500, 550, 600, 650, 700, 750, 800, 850, 900, 950, 978, 1000, 1050, 1100, 1150, 1200, 1300, 1400, 1500, 2000, 3000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 1,100,000, 1,200,000, 1,300,000, 1,400,000, or 1,500,000 nucleic acid precursors.

The nucleic acid sequences of this pool of nucleic acid precursors may differ between all or part of the nucleic acid precursors of the pool. These nucleic acid precursors may differ in nucleotide sequence of the sequence of interest, in primer binding site(s) and/or endonuclease recognition site(s). A pool of nucleic acid precursors may comprise at least 2, 3, 4, 5,10, 50, 100, 150, 200, 250, 300, 350, 400, 450, 500, 550, 600, 650, 700, 750, 800, 850, 900, 950, 978, 1000, 1050, 1100, 1150, 1200, 1300, 1400, 1500 or 2000, 3000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 1,100,000, 1,200,000, 1,300,000, 1,400,000, or 1,500,000 unique sequences. A pool of nucleic acid precursors comprising at least 2 unique sequences is to be understood herein as a pool comprising at least 2 nucleic acid precursors that do not have an identical nucleotide sequence over their whole length, i.e. their nucleotide sequences differ on at least one nucleotide position.

In a preferred embodiment, the initial pool of nucleic acid precursors may contain about 2%, 10%, 15%, 20%, 25%, 30%, 40%, 50%, 60%, 70%, 75%, 80%, 90%, 95%, 98% or 100% unique sequences. The initial pool of nucleic acid precursors is understood herein as the pool of nucleic acid precursors prior to the amplification step. More preferably, the initial pool of nucleic acid precursors may contain about 75% or 100% unique sequences, whereby a pool containing about 75% unique sequences is most preferred. Such pool is typically a pool for the production of probes for a (multiplex) OLA assay, wherein preferably for each SNP 2 distinct allele probes and one locus probe is used, and wherein these probes are present in the ligation assay in the ratio of a first allele probe 1:second allele probe 2:locus probe of 1:1:2, in order to result in equimolar amounts of allele and locus probes. Thus, in a preferred embodiment, the initial pool of nucleic acid precursors may contain unique sequences in a ratio of about 1:1:2. Alternatively, the initial pool of nucleic acid precursors may contain unique sequences in a ratio of about 1:1 (for oligonucleotide production for use in multiplex oligonucleotide-based amplification or OLA assays using only abutting, adjacent or more distantly spaced locus-specific probes).

Preferably the unique sequences of the nucleic acid precursors are selected from the group consisting of SEQ ID NO: 1-SEQ ID NO: 978. In addition, at least one sequence may be selected from the group consisting of SEQ ID NO: 1-326, one sequence may be selected from the group consisting of SEQ ID NO: 327-652 and/or one sequence may be selected from the group consisting of SEQ ID NO: 653-978.

The sequence of the first primer binding site of each of the nucleic acid precursors may be identical for each of the oligonucleotide precursors within the pool. In addition or alternatively, the sequence of the second primer binding site of each of the oligonucleotide precursors in the pool may be identical for each of the nucleic acid precursors within the pool. In addition or alternatively, the first endonuclease recognition site of each of the oligonucleotide precursors in the pool may be identical for each of the nucleic acid precursors within the pool. In addition or alternatively, the second endonuclease recognition site of each of the oligonucleotide precursors in the pool may be identical for each of the nucleic acid precursors within the pool. As indicated earlier herein, in an optional embodiment, the first and second primer and primer binding sites may be identical or highly similar in such a way that the first primer may also anneal to the second primer binding site and vice versa to allow for amplification of the nucleic acid precursor. In an optional embodiment, wherein the first and second endonuclease used in the method of the inventions are restriction enzymes, the first and second endonuclease recognition sites may be identical though in reverse complement orientation to one another. In other words, within this embodiment, the nucleotide sequence of the first endonuclease recognition site within the first strand is the reverse complement of the nucleotide sequence of the second endonuclease recognition site in the first strand.

Optionally, the nucleic acid precursors of a pool are designed in a way that allows for the production of a specific subset of oligonucleotides depending on the selection of one or more particular primer pairs. For instance, particular subsets of nucleic acid precursors within the pool may comprise particular primer binding site combinations. Preferably, these primer binding site combinations comprise one or more primer binding sequences that vary at least in 2, 3, 4, 5, 6 or more nucleotides at the 5′ end of these primer binding sequences (denominated herein as a variable part of the primer binding site), allowing amplification of specific subsets with primers having the corresponding (Watson-Crick) 1, 2, 3, 4, 5, 6 or more nucleotides at their 3′ end.

For example, the first and/or second primer binding sites of two different subsets of nucleic acid precursors comprise a universal part (equal in nucleotide sequence for the two subsets) and a variable part (different in nucleotide sequence for the two subsets). Preferably, this universal part has least 18 nucleotides and the variable part has a length of 1, 2, 3, 4 or more nucleotides. The variable part is located at the 5′ terminal part of the primer binding sequences and the universal part at the 3′ terminal part of the primer binding sequences (see FIGS. 3A-3B and FIGS. 4A-4B for two exemplified embodiments). Upon amplification of such nucleic acid precursors, one or more primers may be used that have selective nucleotides at their 3′-end (being complementary to, and capable of annealing to, the variable part of the primer binding sequence). Presence or absence of such selective nucleotides will determine which subset, or optionally all subsets, of precursors will be amplified. For instance, using primers without selective nucleotides (+0/+0), i.e. primers comprising sequences complementary to the 18 nucleotides long universal part of the primer binding sequence only, will allow for the amplification of both subsets together. Using primer pairs comprising e.g. two selective nucleotides at the 3′-end of both primer pairs (+2/+2) or on one of the primers of a pair (+0/+2 or +2/+0) adjacent to the 18 nucleotides long nucleotides complementary to the universal part of the primer binding sequence will allow for the amplification of either one of the subsets. Hence, in this particular example, the two selective nucleotides of the primer are complementary to the two nucleotides of the variable part, located directly adjacent to the 18 nucleotides of the universal part of the primer binding site.

Hence, a primer pair that anneals to only the universal part of respectively the first and second primer binding sequence allows for the amplification of all subsets, i.e. amplification of the complete initial pool of nucleic acid precursors.

In contrast, a primer pair comprising at least one primer that anneals to (partly or completely) the variable part of the primer binding sequence and, optionally, also anneals to (partly or completely) the universal part of the primer binding sequence allows for the amplification of one or more subsets. It is herein understood that the second primer of this primer pair may anneal to only the universal part of the other primer binding sequence or may anneal (partly or completely) to the variable part of the other primer binding sequence and, optionally, also anneals to (partly or completely) the universal part of the other primer binding sequence.

In a preferred embodiment, the universal part of the primer binding sequence comprises at least 16, 17, 18, 19, 20, 21, 22, 23 or at least 24 nucleotides. In addition, the variable part of a primer binding sequence comprises at least 0, 1, 2, 3, 4, 5, 6, 7, 8, 9 or at least 10 nucleotides.

In addition or alternatively, the nucleic acid precursor may comprise a primer binding site having a variable part and a universal part as detailed herein, wherein a primer may e.g. bind only to variable part to allow for amplification. In this embodiment, the variable part may preferably comprise at least 16, 17, 18, 19, 20, 21, 22, 23 or at least 24 nucleotides. Such relatively long variable part sufficient for a primer to effectively anneal, may also be considered a separate primer binding site on its own. Put differently, the nucleic acid precursors of a pool may thus comprise, next to the first and second primer binding sites, one or two additional primer binding sites (see FIGS. 5A-5B and 6A-6B for exemplified embodiments). More in particular, (the first strand of) a nucleic acid precursor of a pool may comprise the reverse complement of a third primer binding sequence upstream or at the 5′-end of the reverse complement of the first primer binding sequence and/or may comprise a fourth primer binding sequence downstream or at the 3′-end of the second primer binding sequence. The nucleic acid precursors within a pool may be designed such that a particular subset comprises a particular first and second primer binding site combination while a larger subset including this particular subset comprises a particular third and fourth primer binding site combination. It is further herein understood that at least one of the first, second, third and fourth primer binding sites may again comprise a variable part and a universal part as detailed herein, thereby allowing for the amplification of specific subsets through the modification of the variable parts and the use of specific primer pairs.

In addition, the variable part of the primer binding site within the first strand of the precursor may be downstream of the first endonuclease recognition site and/or upstream of the second endonuclease recognition site (exemplified in FIGS. 3A-3B and 5A-5B), such that the first endonuclease cleaves the sugar-phosphate backbone of the first strand downstream of the variable part of the first primer binding site and/or the second endonuclease cleaves the DNA of the first strand upstream of the variable part of the second primer binding site.

The nucleic acid precursor for use in the method of the invention further comprises a first endonuclease recognition site and a second endonuclease recognition site.

The nucleic acid precursor comprises a first endonuclease recognition site designed such that, after duplexing, a first endonuclease cleaves the sugar-phosphate backbone of the first strand immediately upstream of the sequence of interest. The wording “cleaves the sugar-phosphate backbone of the first strand immediately upstream the sequence of interest” means that the sugar-phosphate backbone is cleaved between the 5′-nucleotide of the sequence of interest and the first nucleotide that is upstream (or on the 5′ side) of said 5′-nucleotide. As a result, the 5′-terminal nucleotide of the sequence of interest and the sequence downstream (or on the 3′ side) of said 5′-nucleotide is no longer part of the DNA strand comprising the reverse complement of the first primer binding site and the first endonuclease recognition site.

The nucleic acid precursor comprises a second endonuclease recognition site designed such that, after duplexing, a second endonuclease cleaves the sugar-phosphate backbone of the first strand immediately downstream of the sequence of interest. The wording “cleaves the sugar-phosphate backbone of the first strand immediately downstream the sequence of interest” means that the sugar-phosphate backbone is cleaved between the 3′-nucleotide of the sequence of interest and the first nucleotide that is downstream (or on the 3′ side) of said 3′-nucleotide. As a result, the 3′-nucleotide of the sequence of interest and the sequence upstream of said 3′-nucleotide is no longer part of the DNA strand comprising the second primer binding site and the second endonuclease recognition site. Hence, the first endonuclease recognizing the first endonuclease recognition site of the duplexed precursor, cleaves the DNA immediately upstream the sequence of interest. Similarly, the second endonuclease recognizing the second endonuclease recognition site of the duplexed precursor, cleaves the DNA immediately downstream the sequence of interest.

As detailed herein, the endonuclease cleaves the sugar-phosphate backbone of the first strand either directly upstream (the first endonuclease) or directly downstream (the second endonuclease) the sequence of interest. This may be accomplished by using so-called “outside cutters” known in the art. Such outside cutters may cleave the sugar-phosphate backbone of the first strand directly adjacent to respectively the first and/or second endonuclease recognition sequence within the endonuclease recognition site. Alternatively, outside cutters may cleave the sugar-phosphate backbone at least 1, 2, 3, 4, 5, 6, 7, 8, 9, 10, 11, 12, 13, 14, 15 or 16 nucleotides beyond the recognition sequence of said enzyme. For instance, in case the first endonuclease cleaves 10 nucleotides beyond the endonuclease recognition sequence, there will be 10 nucleotides present between the endonuclease recognition sequence and the sequence of interest. As indicated herein, these nucleotides located in between the endonuclease recognition sequence and the sequence of interest may be part of the first and/or second primer binding site, optionally may constitute the variable part of the first and/or second primer binding site. The first endonuclease and/or second endonuclease may be a nicking endonuclease or a restriction endonuclease. Preferably, the sequence of interest is designed such, and the endonucleases used in the method of the invention are selected such, that the sequence of interest remains intact after the digestion step of the method of the invention.

In case the second strand or its remainder comprising at least the reverse complement of the sequence of interest is separated from the first strand or its remainder comprising at least the sequence of interest, the method of the invention comprises tagging the second strand of the amplified double-stranded precursor. As further detailed herein, this tag is preferably located at the 5′-end of the second strand of the amplified double-stranded precursor, and may be introduced by using a tagged primer in the amplification step. Within this embodiment, the precursor or method is preferably designed such that upon digestion of the amplified double-stranded precursor in the method of the invention, the sugar-phosphate backbone of the second strand from the tag up and including the reverse complement of the sequence of interest remains intact. In addition, the sugar-phosphate backbone of the second strand may be cleaved 3′ of the sequence complementary of the sequence of interest. It is thus preferred that that the sequence complementary to the sequence of interest is not cleaved. However, it is contemplated within the invention that the sugar-phosphate backbone of the sequence that is complementary to the sequence of interest may be cleaved close to its 3′ end, e.g. the sugar phosphate backbone may be cleaved before the last 1, 2, 3, 4, 5, 6, 7, 8, 9 or 10 nucleotides at the 3′ end of the sequence that is complementary to the sequence of interest.

A possible design of the precursor that allows the sugar-phosphate backbone of the second strand from the tag up and including the reverse complement of the sequence of interest to remain intact, is the selection of a second restriction recognition site designed to be recognized by a nicking endonuclease in such an orientation that it only nicks the first strand immediately downstream of the sequence of interest. Said nicking endonuclease is then to be used as a second endonuclease in the digestion step of the method of the invention.

For instance, in case the first endonuclease is Nt.Alwl (New England Biolabs), capable of catalysing a single strand break 4 bases beyond its recognition sequence GGATC (i.e. 5′ . . . GGATCNNNN:N . . . 3′, the first endonuclease recognition site comprises or consists of (in the 5′ to 3′ direction) GGATCNNNN, immediately adjacent to the 5′-nucleotide of the sequence of interest. For instance, in case the second endonuclease is Nb.BsrDI (New England Biolabs), which catalyzes a single strand break directly adjacent to the 5′-end of CATTGC (i.e. 5′ . . . NN:CATTGC . . . 3), the second RE recognition site comprises or consists of (in the 5′ to 3′ direction) CATTGC and is immediately adjacent to the 3′-nucleotide of the sequence of interest.

A possible design of the method that allows the sugar-phosphate backbone of the second strand from the tag up and including the reverse complement of the sequence of interest to remain intact is the selection of a second primer with a chemistry that cannot be cleaved by endonucleases. Such chemistry is known in the art and may be selected from, but is not limited to, chemistry based on phosphorothioate (PS) bonds, methylation (e.g., N6-methyladenosine or mA, 5-methylcytosine or mC, 5-hydroxymethylcytosine or hmC) and Locked nucleic acid (LNA). Within this particular embodiment, the second endonuclease may be a restriction endonuclease that is capable of cleaving the first strand between the 3′-end nucleotide of the sequence of interest and the 5′-end nucleotide of the second endonuclease recognition site, and the second strand between the 5′-end nucleotide of the reverse complement of the sequence of interest and the 3′-end nucleotide of the second endonuclease recognition site or any position on the second strand 5′ of this position. The second primer should be designed such that the second strand of the produced amplicon is inert to cleavage by the selected second (restriction) endonuclease. This may be envisaged by using a modified second primer resulting in an amplicon having endonuclease resistant chemistry on the second strand at the position where the second (restriction) endonuclease would normally cleave.

### Amplification

The method of the invention comprises a step of amplifying the nucleic acid precursor as defined herein by an amplification method using a first primer and a second primer. Amplification of the nucleic acid precursor preferably results in an at least 100 fold, preferably at least 500, 1000 or even at least 5000 fold increase in the abundancy of the nucleic acid precursor. The amplification step in the method of the invention results in the generation of a(n) (amplified) double-stranded nucleic acid precursor.

Any amplification method may be suitable for use in the method of the invention, such as polymerase chain reaction as well as isothermal amplification methods. In case the nucleic acid precursor is amplified using PCR, the use of a high-fidelity DNA polymerase is preferred to reduce the number of misincorporations during the PCR.

Preferably, the amplification method is an isothermal amplification method. Several isothermal amplification methods are known in the art, such as Loop-mediated isothermal amplification (LAMP), Strand displacement amplification (SDA), Nicking enzyme amplification reaction (NEAR), Helicase-dependent amplification (HDA), and Recombinase Polymerase Amplification (RPA) and the invention is described herein is not limited to a single isothermal amplification method. A preferred isothermal amplification method is Recombinase Polymerase Amplification (RPA) or Helicase Dependent Amplification (HDA).

A Helicase Dependent Amplification employs the double-stranded DNA unwinding activity of a helicase to separate strands, enabling primer annealing and extension by a strand-displacing DNA polymerase. HDA is well-known in the art. For example, the HDA method may comprise the following steps as described in U.S. Pat. No. 9,074,248:
Combining a suitable buffer, the nucleic acid precursor; a first and a second primer; a helicase; and deoxynucleotide triphosphates (dNTPs);
incubating the reaction mixture at a temperature that is preferably between about 5 degrees Celsius below the melting temperature of the primer to about 3 degrees Celsius above the melting temperature of the primer; and
obtaining the amplified template nucleic acid.

A particularly preferred amplification method is recombinase polymerase amplification (RPA). RPA is well-known in the art and may be for example performed as described in Piepenburg et al. (2008), WO2003/072805, WO2005/118853, WO2007/096702, WO2008/035205, WO2010/135310, WO2010/141940, WO2011/038197, WO2012/138989 and/or using TwistAmp Basic kit from TwistDX according to manufacturing conditions.

In brief, the nucleic acid precursor(s) as defined herein is/are contacted with a first and a second primer and at least three enzymes, i.e. at least a recombinase, a polymerase and a single-stranded DNA binding protein (SSB), in a suitable buffer for RPA to take place. Preferably, the nucleic acid precursor(s) is/are contacted with the first and second primer prior to the addition of the enzymes. An example of PRA is outlined in detail below. However, the invention is by no means limited to the RPA reaction detailed below and the skilled person understands that variations to this protocol are within the scope of the invention.

For example, 2.4 μL of the first primer (10 μM), 2.4 μL of the second primer (10 μM) and 0.01-0.05 pmol nucleic acid precursors are mixed in H2O to a total volume of 18 μL. Subsequently a buffer may be added, especially in case the enzymes for RPA are in a freeze dried state, e.g. 29.5 μL of a rehydration buffer may be added to the above indicated total volume of 18 μL, which buffer may have the following composition:
0-60 mM Tris, e.g. 25 mM Tris
50-150 mM Potassium Acetate, e.g. 100 mM potassium acetate
0.3-7.5 w/v polyethylene glycol, e.g 5.46% w/v PEG 35 kDa.

Optionally, the rehydration solution (comprising the buffer, primers and nucleic acid precursor(s)) is vortexed and spun down briefly. Subsequently, the total volume of 47.5 μL of rehydration solution may be transferred to a basic RPA freeze-dried reaction pellet, which preferably comprises the following components (wherein the indicated concentrations are as before freeze drying or as after reconstitution):
at least one recombinase (e.g. 100-350 ng/μL uvsX recombinase, such as 260 ng/μL, preferably bacteriophage T6 UvsX recombinase);
at least one single stranded DNA binding protein (150-800 ng/μL gp32, such as 254 ng/μL, preferably bacteriophage Rb69 gp32);
at least one DNA polymerase (e.g. 30-150 ng/μL Bacillus subtilis Pol I (Bsu) polymerase or S. aureus Pol I large fragment (Sau polymerase), such as 90 ng/μL);
dNTPs or a mixture of dNTPs and ddNTPs (150-400 μM dNTPs, such as 240 μM);
a crowding agent (e.g., polyethylene glycol, preferably 1.5-5% w/v PEG 35 kDa, such as 2.28% w/v PEG 35 kDa, optionally in combination with 2.5%-7.5% weight/volume of trehalose, such as 5.7% w/v trehalose);
a buffer (e.g. 0-60 mM Tris buffer, such as 25 mM Tris);
a reducing agent (e.g. 1-10 mM DTT, such as 5 mM DTT);
ATP or ATP analog (e.g. 1.5-3.5 mM ATP, such as 2.5 mM ATP);
optionally at least one recombinase loading protein (e.g. 50-200 ng/μL uvsY, preferably bacteriophage Rb69 uvsY, such as 88 ng bacteriophage Rb69 uvsY);
phosphocreatine (e.g. 20-75 mM, such as 50 mM phosphocreatine); and
creatine kinase (e.g. 10-200 ng/μL, such as 100 ng/μL).

The reaction mixture may further comprise 50-200 ng/μL of either exonuclease III (exoIII), endonuclease IV (Nfo) or 8-oxoguanine DNA glycosylase (fpg).

Magnesium may be added to the reaction mixture to start the RPA reaction, e.g. magnesium acetate may be added to an end concentration in the reaction mixture of 8-16 mM (for example 2 μL 280 mM magnesium acetate may be added to the above exemplified reaction volume of 47.5 μL). Optionally, the magnesium acetate is already present in the reaction mixture, i.e. is not added subsequently but e.g. contacted to the nucleic acid precursor(s) together with the other constituents of the rehydration solution defined above. The reaction is incubated until a desired degree of amplification is achieved. After contacting the oligonucleotide precursors with these enzymes, primers and buffer components as indicated above, the mixture is preferably incubated for about 1 hour at about 37° C. (preferably between 25° C. and 42° C.). Preferably, RPA results in amplification of the nucleic acid precursor of at least 100 fold, preferably at least 200, 300 or even at least 400 fold, e.g. about 500 fold.

Other protocols for RPA may be equally suitable for amplification of the nucleic acid precursor. More in particular, other recombinases may be used such as, but not limited to E. coli RecA or any homologues protein or protein complex from any phyla (e.g. Rad51) or RecT or RecO, or Uvx such as Aeh1 Uvx, T4 UvsX, T6 UvsX and Rb69 Uvx. The polymerase may be an eukaryotic or a prokaryotic polymerase. Prokaryotic polymerase include, at least, E. coli pol I, E. coli pol II, E. coli pol III, E. coli pol IV and E. coli polV. Eukaryotic polymerase include, for example, multiprotein polymerase complexes selected from the group consisting of pol-, pol-β, pol-δ, and pol-ε. A suitable polymerase may be E. coli PolV or a homologues polymerase of other species. A further suitable a single-stranded DNA binding protein (SSB), may be E. coli gp32, or Aeh1 gp32, T4 gp32, Rb69 gp32. Suitable enzyme concentration to be used are: 20 μM recombinase, about 1-10 μM SSB and about 1-2 μM polymerase. A further optional crowding agent (apart from polyethylene glycol and/or trehalose) is, but is not limited to, polyethylene oxide, polyvinyl alcohol, polystyrene, Ficoll, dextran, PVP and albumin. In a preferred embodiment, the crowding agent has a molecular weight of less than 200,000 daltons. Further, the crowding agent may be present in an amount of about 0.5% to about 15% weight to volume (w/v).

The primers used for amplification of the nucleic acid precursor anneal to the nucleic acid precursor to such an extent to allow for the primer-extension for amplification using e.g. RPA or PCR. In particular, the first primer anneals (only) to the first primer binding sequence and the second primer anneals (only) to the second primer binding sequence.

In a preferred embodiment, the first primer is fully complementary to the first primer binding sequence and the second primer is fully complementary to the second primer binding sequence. In case of a primer binding site with variable part as defined herein the primer may be fully complementary to only the universal part of the primer binding sequence and optionally part of the variable part of the primer binding sequence. Alternatively, the primer may be fully complementary to only the variable part of the primer binding sequence and optionally part of the universal part of the primer binding sequence. Similarly, the primer may be partly complementary to the variable part of the primer binding sequence and partly complementary to the universal part of the primer binding sequence.

In addition, the first and/or the second primer may further comprise an additional sequence that is present 5′ of the sequence that is complementary to the primer binding sequence. Preferably, said additional sequence may consist of 1, 2, 3, 4, 5, 6, 7, 8, 9, 10 or 15 additional nucleotides 5′ of the complementary sequence. As indicated herein above, the first strand is to be understood herein as being the strand comprising the sequence of interest, either of the nucleic acid precursor or of the amplicon obtained in step b) of the method of the invention. Likewise, the second strand is to be understood herein as the strand of the nucleic acid precursor or of the amplicon obtained in step b) of the method of the invention, complementary to the first strand. As is understood by the skilled person, in case the first and second primer comprise additional nucleotides at their 5′ end as indicated herein, the strands of the amplicon obtained in step b) of the method of the invention will be longer than the respective strands of the nucleic acid precursor.

The length of the first primer and/or second primer is preferably about 12, 13, 14, 15, 16, 17, 18, 19, 20, 21, 22, 23, 24, 25, 26, 27, 28, 29 or 30 nucleotides. The first primer and the second primer may have the same or a different length. In a preferred embodiment, the length of the first primer is preferably about 12, 13, 14, 15, 16, 17, 18, 19, 20, 21, 22, 23, 24, 25, 26, 27, 28, 29 or 30 nucleotides and the length of the second primer is preferably about 12, 13, 14, 15, 16, 17, 18, 19, 20, 21, 22, 23, 24, 25, 26, 27, 28, 29 or 30 nucleotides. Preferably, the first and second primers are designed such that they are complementary to at least 18 consecutive nucleotides of the first and second primer binding sequence, respectively.

As detailed herein, the second primer may comprise an affinity tag conjugated to the nucleotide at the 5′-end. Any affinity tag that can be conjugated to the 5′-end of a nucleotide is suitable for use in the preferred embodiment of the invention, wherein the second strand or part thereof comprising the reverse complement of the sequence of interest is separated from the first strand or part thereof comprising the sequence of interest.

Alternative to a 5′ end conjugate tag, the affinity tag may be located internally within the sequence of the second primer. For example, the second primer may comprise one or more biotin-modified thymidine residues.

The term “affinity tag” as used herein, refers to a moiety that can be used to separate a molecule to which the affinity tag is attached from other molecules that do not contain the affinity tag. In certain cases, an “affinity tag” may bind to the “capture agent,” where the affinity tag specifically binds to the capture agent, thereby facilitating the separation of the molecule to which the affinity tag is attached from other molecules that do not contain the affinity tag. Examples of affinity tags include 6-histaminylpurine (as e.g. described in Min and Verdine, 1996 Nucleic Acids Research 24:3806-381), a polynucleotide-tail such as a poly A tail capable of being attached to a solid support having a poly T complement, or biotin capable of attaching to e.g. streptavidin or avidin on a solid support, wherein biotin is the most preferred.

As used herein, the term “biotin” refers to an affinity agent that includes biotin or a biotin analogue such as dual-biotin, desthiobiotin, PC-biotin, oxybiotin, 2′-iminobiotin, diaminobiotin, biotin sulfoxide, biotin azide, biocytin, etc. Preferably, biotin moieties bind to streptavidin with an affinity of at least 10⁻⁸M. A biotin affinity agent may also include a linker, e.g., -LC-biotin, -LC-LC-Biotin, -SLC-Biotin or -PEGn-Biotin where n is 3-12.

In a preferred method of the invention, the second primer comprises an affinity tag.

The affinity tag can be present on at least the second primer. It is further understood herein that the affinity tag can be present on both the first primer and the second primer. Alternatively, the affinity tag is not present on the first primer, e.g. it is only present on the second primer.

Amplification of the nucleic acid precursor thus results in an amplified double-stranded nucleic acid precursor comprising at least one tag, wherein the tag is on the strand comprising the sequence complementary to the first strand. The amplified double-stranded nucleic acid precursor can further also comprise a tag on the first strand, preferably at the 5′ end of the first strand. The tag on the first strand and the tag on the second strand can be the same or different type of tags. As a non-limiting example, the tag on the first strand and the second strand can be biotin.

In a preferred embodiment, amplification of the nucleic acid precursor results in an amplified double-stranded nucleic acid precursor which comprises a tag only on the strand comprising the sequence complementary to the first strand. In particular, the strand comprising the sequence complementary to the first strand comprises the tag at the 5′-end. Most preferably, the complementary strand comprises biotin at the 5′ end.

Alternatively, the biotin moiety may be present internally, e.g. within the sequence of the complementary strand, e.g. when the second primer comprises one or more biotin-modified thymidine residues.

Preferably the amplified double-stranded precursor is purified prior to binding the solid support. Preferably, the purification results in separating the amplified and tagged precursor from the (unused) tagged second primer. The purification of the double-stranded precursor may be performed using any method known in the art to purify amplified nucleic acid products. Preferred purification methods include, but are not limited to, column purification (e.g. QIAquick PCR purification columns) and separation on an agarose or acrylamide gel.

### Digestion

The method of the invention comprises a step of digesting the amplified double-stranded precursor with a first restriction or nicking endonuclease recognizing the first endonuclease recognition site and with a nicking endonuclease recognizing the second endonuclease recognition site. Digestion with the first and second endonuclease results in the production of an amplified double-stranded nucleic acid precursor with cleavages of the sugar-phosphate backbone immediately up- and downstream of the sequence of interest.

The first endonuclease binding to the first endonuclease recognition site cleaves either both sugar-phosphate backbones (being a restriction endonuclease) or cleaves only one of the two sugar-phosphate backbones (being a nicking endonuclease). In case the first endonuclease is a nicking endonuclease, the first endonuclease recognition site is oriented such that the nicking endonuclease cleaves the first strand immediately upstream of the sequence interest.

As indicated herein, the first endonuclease binding to the first endonuclease recognition site preferably is an outside-cutter, e.g. cleaving the sugar phosphate backbone immediately (directly) adjacent or at least 1, 2, 3, 4, 5, 6, 7, 8, 9, 10, 11, 12, 13, 14, 15, or 16 nucleotides beyond the endonuclease recognition sequence as detailed above. Examples of such enzymes are “Type IIS restriction enzymes”. The first endonuclease cleaves at least the sugar-phosphate backbone directly immediately upstream (5′) of the sequence of interest. Thus, the first endonuclease cleaves i) the first DNA strand; or ii) the first and the second DNA strand.

Hence, the first endonuclease may be an outside cutter cleaving both strands of DNA, i.e. a restriction endonuclease, or only one strand of DNA, i.e. a nicking endonuclease. In both instances, the first endonuclease recognition site is designed such that the outside cutter binds the site in an orientation that allows for the endonuclease to cleave the sugar-phosphate backbone of the first strand 3′ of the endonuclease recognition site. More preferably, the first endonuclease recognition site is designed such that the outside cutter binds the site in an orientation that allows for the endonuclease to cleave the sugar-phosphate backbone of the first strand 3′ of the endonuclease recognition site and immediately upstream of the sequence of interest.

Non-limiting examples of endonucleases suitable for use as first endonucleases are given below.

Non-limiting examples of endonucleases cleaving both strands of DNA are suitable for use as first endonuclease are: MnII, BspCNI, BsrI, BtsIMutI, HphI, HpyAV, MboII, AcuI, BciVI, BmrI, BpmI, BpuEI, BseRI, BsgI, BsmI, BsrDI, BtsαI, BtsI, EciI, MmeI, NmeAIII, AsuHPI, Bse1I, BseGI, BseMII, BseNI, BsrSI, BstF5I, Hin4II, TscAI, TseFI, TspDTI, TspGWI, ApyPI, Bce83I, BfiI, BfuI, BmuI, BsaMI, BsbI, BscCI, Bse3DI, BseMI, BsuI, CchII, CchIII, CdpI, CjeNIII, CstMI, Eco57I, Eco57MI, GsuI, Mva1269I, PctI, PIaDI, PspPRI, RdeGBII, RleAI, SdeAI, TagII, TsoI, Tth111II, WviI, AquII, AquIV, DraRI, MaqI, PspOMII, RceI, RpaB5I, RpaBI, RpaI, SstE37I and RdeGBIII.

A preferred nicking endonuclease for use as a first endonuclease may be selected from the group consisting of Nt.Alwl, Nt.BsmAI, Nt.BstNBI and Nt.BspQI (New England Biolabs). A particularly preferred first endonuclease is Nt.Alwl.

The skilled person understands how to select a first endonuclease and how to design the first endonuclease recognition site to ensure that the endonuclease cleaves at least the sugar-phosphate backbone immediately upstream of the 5′ nucleotide of the sequence of interest.

The amplified double-stranded precursor is additionally digested with a second endonuclease recognizing the second endonuclease recognition site (the second endonuclease). The second endonuclease may be an outside cutter cleaving both strands of DNA, i.e. a restriction endonuclease, or only one strand of DNA, i.e. a nicking endonuclease. In both instances, the second endonuclease recognition site is designed such that the outside cutter binds the site in an orientation that allows for the endonuclease to cleave the sugar-phosphate backbone of the first strand 5′ of the endonuclease recognition site, immediately 3′ after the last nucleotide of the sequence of interest. Thus, the second endonuclease recognition site is designed such that the outside cutter binds the site in an orientation that allows for the endonuclease to cleave the sugar-phosphate backbone of the first strand 5′ of the endonuclease recognition site and immediately downstream of the sequence of interest.

As indicated herein, the second endonuclease binding to the second endonuclease recognition site preferably is an outside-cutter, e.g. cleaving the sugar-phosphate backbone immediately (directly) adjacent or at least 1, 2, 3, 4, 5, 6, 7, 8, 9, 10, 11, 12, 13, 14, 15, or 16 nucleotides upstream of the endonuclease recognition sequence as detailed above. In case the second endonuclease is a restriction endonuclease, it may be selected from the same list as indicated herein above as suitable endonucleases cleaving both strands of DNA suitable for use as first endonuclease.

As indicated herein, in particular embodiments, it is preferred that the second endonuclease recognizing and binding to the second endonuclease recognition site is a nicking endonuclease, i.e. the endonuclease cleaves only the first strand of the double-stranded DNA, immediately downstream of the (terminal) 3′ nucleotide of the sequence of interest.

A nicking endonuclease suitable for use as a second endonuclease may be selected from the group consisting of Nb.BsrDI, Nb.BtsI, AspCNI, BscGl, BspNCI, FinI, TsuI, UbaF11I, BspGI, DrdII, PfI1108I, UbaPI, EcoHI, UnbI or Vpac11AI. A particularly preferred second endonuclease is Nb.BsrDI.

The restriction and/or nicking of the amplified nucleic acid precursor is performed by contacting the (amplified) precursor with the enzyme or enzymes in a suitable buffer at a suitable temperature according to manufacturer's instructions. The first and second endonuclease may be added simultaneously. Alternatively, the precursor may be contacted with the first (or second) endonuclease, optionally the precursor is purified, and subsequently the second (or first) endonuclease is added in the appropriate buffer. After restriction using the first and second endonuclease, the restricted precursor may be purified.

### Immobilization

In a preferred embodiment of the method of the invention, the second strand of the amplified double-stranded nucleic acid precursor comprises an affinity tag which is brought into contact with a capture agent, wherein said capturing agent is preferably comprised on a solid support. A suitable capture agent is dependent on the affinity tag. For example if the nucleic acid comprises a biotin tag, the capture agent may be e.g. streptavidin or avidin. Further possible tags may be His-tag, DNP (2,4-dinitrophenyl) or Digoxigenin (DIG), wherein the capture agent may be anti-His antibody, anti-DNP antibody or anti-DIG antibody, respectively. Similarly, if the affinity tag comprises a polynucleotide tail, the capture agent may be its complementary sequence.

The solid support or gel may comprise the capture agent. Preferably, the capture agent is present on a solid support. Binding of the affinity tag to the capture agent may thus result in immobilization of the amplified tagged double-stranded nucleic acid precursors, and/or immobilization of tagged single-stranded oligonucleotides, to the solid support. Any solid support that is suitable for the immobilization of a tagged nucleic acid is suitable for use in the method of the invention.

A solid support with internal or external surface may be in any suitable format including particles, powders, sheets, beads, filters, flat substrate, tubes, tunnels, channels, metallic particles etc. The support can be porous, which may provide internal surface for the immobilization of nucleic acid precursor to occur. Preferred materials do not interfere with the interaction between the tagged nucleic acid precursor and the capture agent. Suitable materials may include, but are not limited to paper, glasses, ceramics, metals, metalloids, polacryloylmorpholine, various plastics and plastic copolymers such as Nylon™, Teflon™, polyethylene, polypropylene, poly(4-methylbutene), polystyrene, polystyrene, polystyrene/latex, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and controlled-pore glass (Controlled Pore Glass, Inc., Fairfield, N.J.), aerogels and the like, and any materials generally known to be suitable for use in affinity columns (e.g. HPLC columns).

The solid support may be in the form of beads (or other small objects having suitable surfaces) that are identifiable individually or in groups. Preferably, the solid support may also be separable according its magnetic properties. Thus in a preferred embodiment of the invention the affinity tag is or comprises biotin and the solid support comprises streptavidin. Preferably the solid support is a bead and wherein more preferably the bead is a magnetic bead. A particularly preferred solid support is are DynaBeads® or the like.

In a particularly preferred embodiment, the immobilization may be performed by incubation with functionalized (para)magnetic particles (or beads), wherein the particles are functionalized in that their surface comprises the binding partners of the tags of the second primers as defined herein. In case such tag is biotin, the particles may be functionalized with streptavidin. The particles (or beads) preferably are about 1-5 μm in diameter and may comprise one or more of the following characteristics: Hydrophilic bead surface, based on carboxylic acid beads, diameter about 1.05 μm, isoelectric point pH 5.2, medium charged (−35 mV (at pH 7), iron content (Ferrites) about 26% (37%), and a low aggregation.

### Denaturation

In a preferred embodiment of the invention the amplified, and preferably digested, double-stranded nucleic acid precursor is denatured, e.g. the first strand is separated from the second complementary strand. The skilled artisan is familiar with the various methods to denature double-stranded DNA. Such methods may include, but are not limited to, exposure of the double-stranded DNA to heat and/or chemical agents. Preferably the denaturing in the method of the invention comprises chemical denaturing. Preferred chemical agents to denature the DNA are e.g. formamide, guanidine, sodium salicylate, dimethyl sulfoxide (DMSO), propylene glycol, urea or an alkaline agents. Preferably, the chemical denaturing is by increasing the pH by the addition of a strong base. Preferably, the strong is base is an alkali hydroxide. In particular, a suitable strong base (or combination thereof) for increasing the pH may preferably be selected from the group consisting of NaOH, LiOH, KOH, RbOH, CsOH, Mg(OH)₂, Ca(OH)₂, Sr(OH)₂ and Ba(OH)₂. Most preferably, the strong base for denaturing the double-stranded nucleic acid precursor in the method of the invention is the alkali hydroxide NaOH.

The strong base, may preferably be added at an end concentration of about 0.5-1.5 M, preferably of about 0.7-1.2 M, or preferably the end concentration is about 0.7, 0.8, 0.9, 1.0, 1.1, or 1.2M. Most preferably the end concentration is about 1 M.

The double-stranded precursor may be incubated with the strong base for about 1-30 minutes, preferably 5-15 minutes, or preferably the double-stranded precursor is incubated for at least about 1, 2, 3, 4, 5, 6, 7, 8, 9, 10, 11, 12, 13, 14 or 15. Most preferably, the double-stranded precursor may be incubated with the strong base for about 10 minutes.

After denaturing the double-stranded precursor, an acid may be added to neutralize the reaction. This neutralizing reaction may be performed before or after the solid support is separated from the single-stranded oligonucleotide as described below. Preferably, the neutralizing reaction is performed after the separation. Any acid may be suitable to neutralize. Preferably the acid is a strong acid such as HCl, HI, HBr, HClO₄, HNO₃ or H₂SO₄, whereby HCl is the most preferred.

The strong acid is preferably added at an end concentration of about 0.5-1.5 M, or about 0.7-1.2 M or preferably the end concentration is about 0.7, 0.8, 0.9, 1.0, 1.1, or 1.2 M. Most preferably the end concentration is about 1 M. Preferably, acid is added in equimolar amounts as base used for denaturation, thereby resulting in complete neutralization.

### Separation

The preferred method of the invention wherein the second strand or part thereof comprising the reverse complement of the sequence of interest is separated from the first strand or part thereof comprising the sequence of interest, comprises a step of removing the solid support to obtain a single-stranded oligonucleotide having the sequence of interest.

The solid support comprises the capture agent. In the method of the invention, the capture agent (e.g. streptavidin) has captured the affinity tag (e.g. biotin) and the affinity tag is preferably coupled to the complementary (second) strand of the nucleic acid precursor. Hence, separating the solid support from the single-stranded oligonucleotide also entails separating the (tagged) complementary strand from the single-stranded oligonucleotide.

Separating the solid support from the single-stranded oligonucleotide can be done using any conventional method known in the art and the method will be dependent on the type of solid support that is used. E.g. in case the solid support comprises small particles, these particles may be spun down and preferably the supernatant comprising the oligonucleotide may be transferred to another vial.

In case the solid support comprises magnetic or paramagnetic beads, the solid support may be removed by magnetic separation, e.g. by placing a magnet in close vicinity of the solid support.

### Purification

The single-stranded oligonucleotide that is obtained after removing the solid support may optionally be further purified. Hence, in a preferred embodiment of the invention, the method further comprises a step g) of purifying the single-stranded oligonucleotide.

The purification can be done using any conventional oligonucleotide purification method that is known in the art. A preferred purification method is affinity purification, such as (mini-)column-purification. However other purification methods, e.g. separation on an agarose or acrylamide gel, may be equally suitable for purifying the single-stranded oligonucleotide.

### Labelling

The single-stranded oligonucleotide that is obtained in the method of the invention may subsequently be labelled. For example, the produced single-stranded oligonucleotide may be labelled with a fluorophore, a hapten, an affinity ligand or a radioactive moiety. Alternatively, the produced single-stranded oligonucleotide is not labelled.

The invention as detailed herein is particularly suitable for the production of single-stranded DNA oligonucleotides. Nonetheless, the method may also result in the production of an RNA molecule, e.g. for use in genome-editing approaches, such as CRISPR-Cas guide RNA (as described for example in Mali et al, 2013, Nature Methods, 10(10):957-63 and Cong et al 2013, Science, 339(9121):819-23). For example for the production of an RNA molecule, the method of the invention may be modified as follows: Step a) of the method as detailed herein comprises at least one (single- or double-stranded) nucleic acid precursors comprising the following elements in the 5′ to 3′ direction: (1) the first primer binding site, (2) a sequence of interest, and (3) the second primer binding site. The sequence of interest may comprise the sequence encoding the RNA and may further comprise a promoter for transcribing RNA, preferably a T7 promoter. Preferably, the promoter is operably linked to the sequence of interest. After obtaining the (optionally un-tagged) double-stranded oligonucleotides in step b), wherein optionally the second primer does not comprise a tag. RNA can be transcribed from the duplex DNA using conventional methods known in the art, such as using a T7 promoter (and having Mg²⁺ as a cofactor).

### Further Aspects of the Invention

In a second aspect, the invention pertains to a nucleic acid precursor comprising a first strand, wherein the first strand comprises the following elements in a 5′ to 3′ direction:
(1) a first primer binding site;
(2) an a first endonuclease recognition site;
(3) the sequence of interest;
(4) a second endonuclease recognition site; and,
(5) a second primer binding site.

Preferably, a first primer can selectively anneal to only the first primer binding sequence as further detailed in the first aspect of the invention and a second primer can selectively anneal to only the second primer binding sequence as further detailed in the first aspect of the invention. Optionally the first and second primers and first and second primer binding sites are identical or similar in such a way that the first primer anneals to the second primer binding sequence and vice versa, to allow for amplification of the nucleic acid precursor.

Preferably, the first endonuclease recognition site is designed such that, after duplexing, a first endonuclease cleaves the sugar-phosphate backbone of the first strand immediately upstream of the sequence of interest.

Preferably, the second endonuclease recognition site is designed such that, after duplexing, a nicking endonuclease cleaves the sugar-phosphate backbone of the first strand immediately downstream of the sequence of interest.

Preferably, the precursor is designed such that the sugar-phosphate backbone of the sequence of interest (i.e. from the 5′ nucleotide of the sequence of interest to the 3′ nucleotide of the sequence of interest) is not cleaved by the first and second endonuclease used in the method of the invention.

Preferably, the sequence of interest does not comprise the first and the second endonuclease recognition sites or reverse complement thereof.

The nucleic acid precursor may be a single- or a double-stranded nucleic acid precursor. If the nucleic acid precursor is double-stranded, the precursor comprises a second strand that is complementary to the first strand. The precursor is further specified as detailed herein above. In the most preferred embodiment, the nucleic acid precursor has a sequence selected from the group consisting of SEQ ID NO: 1-978.

The nucleic acid precursor may be double-stranded. In a further preferred embodiment, the double-stranded nucleic acid precursor comprises an affinity tag.

Preferably, the affinity tag is located at the 5′ end of the second strand. For example, the 5′ nucleotide of the complementary strand may comprise a biotin tag or a polynucleotide-tail. Preferably, the complementary strand comprises a biotin tag at the 5′ end of the second strand, i.e. is biotinylated at the 5′ end. The biotin moiety may be conjugated to the 5′ nucleotide using any conventional method known in the art.

Alternatively, the affinity tag is located internally within the complementary sequence. Preferably, such internal affinity tag is located on the second strand 5′ of second endonuclease recognition site (i.e. 5′ of the sequence that is reverse complement to the endonuclease recognition site of the first strand). More preferably, such internal affinity tag is located on the second strand at the second primer binding site (i.e. on the sequence that is reverse complement to the second primer binding sequence of the first strand). A preferred example of such internal affinity tag is a biotin-modified thymidine residue.

Preferably, the double-stranded nucleic acid precursor does not comprise an affinity tag at the 3′ end and/or 5′ end of the first strand. Preferably, the double-stranded nucleic acid precursor comprises an affinity tag only at the only at the 5′ end of the second strand.

In a third aspect, the invention concerns a solid support comprising the double-stranded nucleic acid precursor as defined herein above. The solid support is further specified as detailed above. Preferably, the double-stranded nucleic acid precursor is bound to the solid support by means of affinity-capture. The first strand and the second strand of the double-stranded nucleic acid precursor may have a fully intact sugar-phosphate backbone. Alternatively, the first strand of the precursor may comprise at least one or two cleavages of the phosphodiester bond and the second strand of the precursor has a fully intact sugar-phosphate backbone or alternatively, the first strand of the precursor may comprise at least one or two cleavages of the phosphodiester bond and the second strand of the precursor has at most one cleavage of the phosphodiester bond.

In a further embodiment, the solid support comprises the single-stranded second strand, i.e. the strand complementary to the first strand as defined herein above.

In a fourth aspect, the invention pertains to a kit containing elements for use in a method of the invention. Such a kit may comprise a carrier to receive therein one or more containers, such as tubes or vials

Preferably, the kit comprises at least one of the following:
a container (1) comprising a second (nicking) endonuclease and optionally the first endonuclease as defined herein above;
a container (2) comprising enzymes for use in the amplification step as defined herein above;
a container (3) comprising a solid support for affinity purification as defined herein above; and
a container (4) comprising a chemical for denaturation as defined herein above.

In a preferred embodiment, the kit comprises container (1) and (2), or (1) and (3), or (1) and (4). In another preferred embodiment, the kit comprises container (2) and (3), or (2) and (4), or (3) and (4). In another preferred embodiment, the kit comprises container (1), (2) and (3), or (1), (2) and (4), or (1), (3) and (4). In another preferred embodiment, the kit comprises container (2), (3) and (4), or (1), (2), (3) and (4). In the most preferred embodiment, the kit comprises container (1), (2), (3) and optionally container (4).

In a further preferred embodiment, the kit further as defined above comprises a container (5) comprising the first and/or tagged second primer as defined herein above. Alternatively, the first and/or second tagged primer may be comprised within the container (2) comprising the enzymes for use in the amplification step.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

In a fifth aspect, the invention pertains to the use of a nucleic acid precursor as defined herein or a kit of parts as defined herein for the production of one or more single-stranded oligonucleotides. The produced single-stranded oligonucleotides may consist or comprise a sequence of interest as defined herein above.

In a sixth aspect, the invention concerns the use of a nucleic acid precursor as defined herein or a kit of parts as defined herein for the amplification of one or more single-stranded oligonucleotides. The produced single-stranded oligonucleotides may consist of or comprise a sequence of interest as defined herein above.

### BRIEF DESCRIPTION OF THE DRAWINGS

FIG. 1: Schematic representation of a preferred embodiment of the method of the invention. PBS1 is the first primer binding site, PBS2 is the second primer binding site, ES1 is the first endonuclease recognition site and ES2 is the second endonuclease recognition site. The reverse primer may comprise a tag (black circle). The solid support (big circle) can capture the tagged, amplified and nicked nucleic acid precursor.

FIGS. 2A-2B: Two exemplified nucleic acid precursors of the invention. FIG. 2A) The first endonuclease recognition site may be (partly or fully) comprised within the first primer binding site and the second endonuclease recognition site may be (partly of fully) comprised within the second primer binding site. FIG. 2B) A nucleic acid precursor whereby the elements are five distinct elements. Abbreviations and symbols are as indicated for FIG. 1. Arrows are primers and the reverse primer may comprise a tag (black circle).

FIGS. 3A-3B: Exemplified nucleic acid precursor of the invention. The primer binding site (PBS) may overlap with the endonuclease recognition site (ES, black). In addition, the primer binding site may comprise a universal part (black and white) and a variable part (grey). FIG. 3A) Amplification using a primer pair that is complementary to the universal part and variable part of the primer binding sites allows for the amplification of a specific subset of nucleic acid precursors. FIG. 3B) Amplification using a primer pair complementary to only the universal parts allows for the amplification of the complete pool of nucleic acid precursors. Abbreviations and symbols are as indicated for FIG. 1 and FIGS. 2A-2B.

FIGS. 4A-4B: Exemplified nucleic acid precursor of the invention. The nucleic acid precursor may comprise five distinct elements. The primer binding site may comprise a universal part (white) and a variable part (grey). FIG. 4A) Amplification using a primer pair that is complementary to the universal part and variable part of the primer binding sites allows for the amplification of a specific subset of nucleic acid precursors. FIG. 4B) Amplification using a primer pair complementary to only the universal part (white) allows for the amplification of the complete pool of nucleic acid precursors. Abbreviations and symbols are as indicated for FIG. 1 and FIGS. 2A-2B.

FIGS. 5A-5B: Exemplified nucleic acid precursor of the invention. The primer binding site (PBS) may overlap with the endonuclease recognition site (ES, black). The primer binding site may comprise a variable part (grey) and a universal part (black and white). FIG. 5A) Amplification using a primer pair that is only fully complementary to the variable part and ES allows for the amplification of a specific subset of nucleic acid precursors. FIG. 5B) Amplification using a primer pair complementary to only the universal part (white) allows for the amplification of the complete pool of nucleic acid precursors. Abbreviations and symbols are as indicated for FIG. 1 and FIGS. 2A-2B.

FIGS. 6A-6B: Exemplified nucleic acid precursor of the invention. The nucleic acid precursor may comprise five distinct elements. The primer binding site may comprise a variable part (grey) and a universal part (white). FIG. 6A) Amplification using a primer pair that is only fully complementary to the variable part allows for the amplification of a specific subset of nucleic acid precursors. FIG. 6B) Amplification using a primer pair complementary to only the universal part (white) allows for the amplification of the complete pool of nucleic acid precursors. Abbreviations and symbols are as indicated for FIG. 1 and FIGS. 2A-2B.

FIG. 7: Result Tapestation D1000 (Agilent): 1 μL of 200 μL un-purified PCR sample total was checked and 1 μL of 50 μL total (purified) RPA sample was checked.

FIG. 8: Result Tapestation D1000: clear visible double stranded amplification products of 102 bp are detected (1 μL of 100 μL total was checked), which are expected to be the amplified probe precursors. The size difference is very likely due to incorrect sizing of the Tapestation system.

FIG. 9: Purification with biotin, result Agilent Small RNA kit (1 μL of ¼ diluted sample of 40 μL total was checked). The recovered DNA corresponded to the expected single-stranded 55-63 nt probes. The size difference is very likely due to incorrect sizing of the array system.

FIG. 10: Result Small RNA Agilent of comparative experiments.

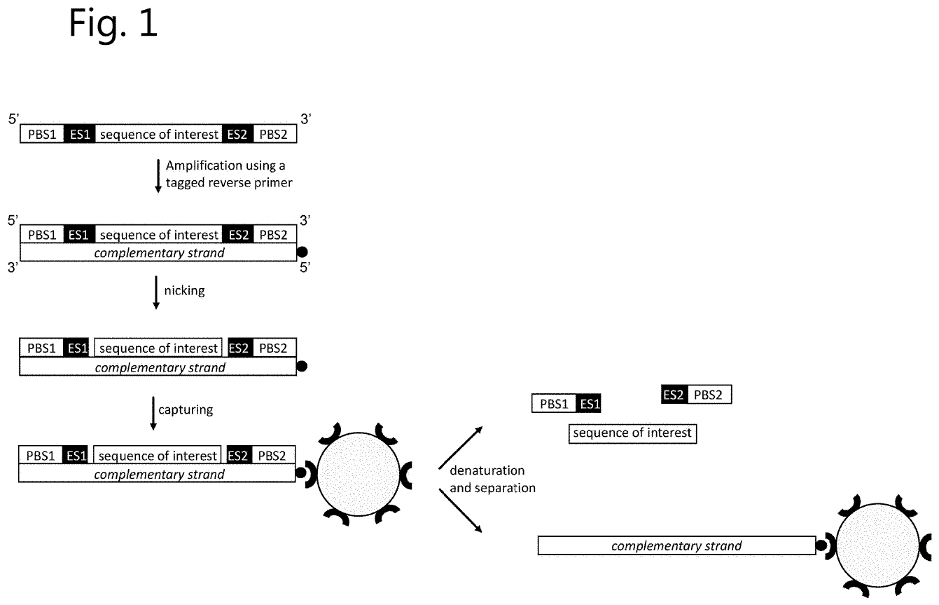

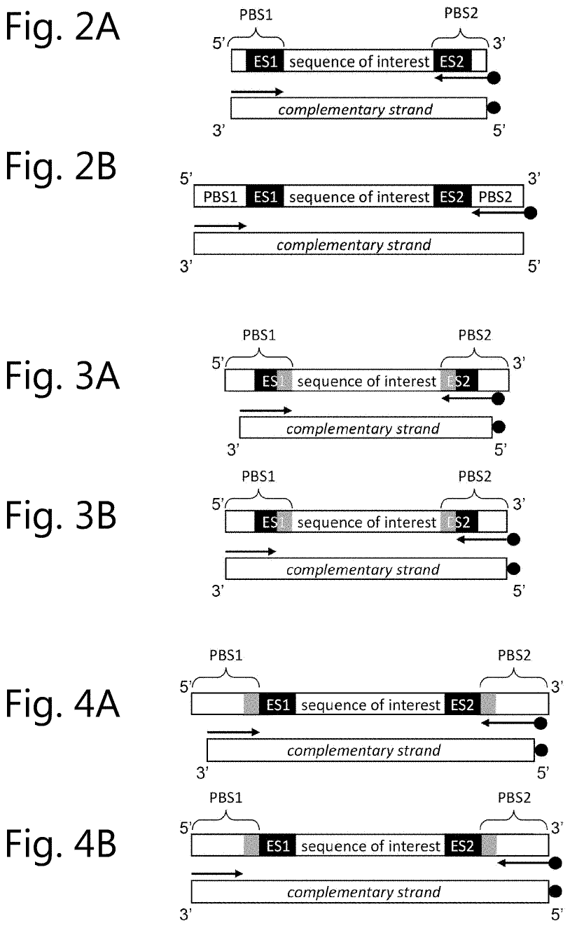

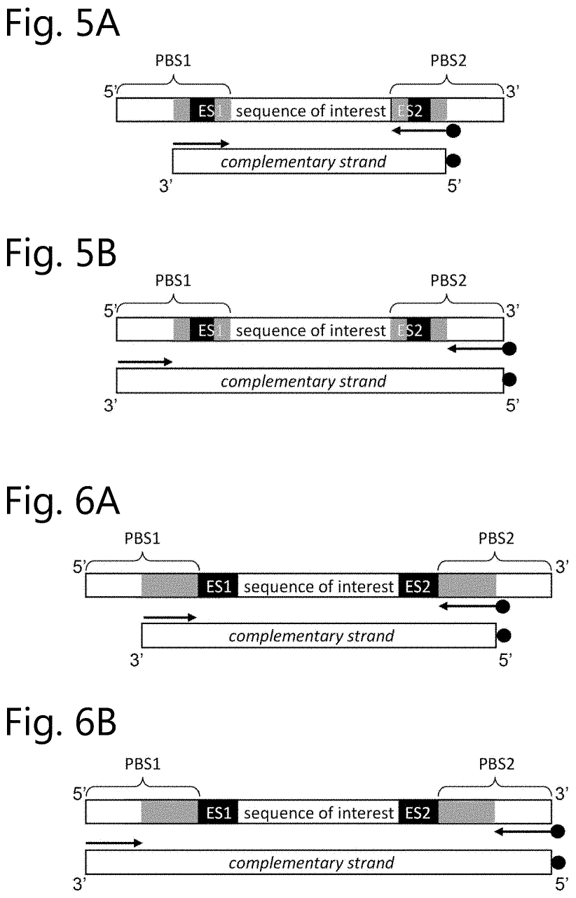

### EXAMPLES

Initial experiments on probe amplification of a multiplex of 9 probe precursors using a method comprising PCR amplification, amplicon nicking, purification of the nicked amplicons by acrylamide-gel separation, and subsequent heat-denaturation to release of the probes, did not result in a satisfying probe yield. This problem was overcome using biotin-bead purification instead of acrylamide-gel separation, in combination with chemical denaturation instead of heat denaturation. However, increasing the multiplex level to 3912 probes again resulted in low yield and hetero-duplex formation (see Example 1). These problems were overcome by using an isothermal amplification method instead of PCR, together with using biotin-bead for amplicon purification and chemical denaturation for probe release. This amplification method resulting in high yield without hetero-duplex formation is described in detail in Examples 2 and 3.

### Example 1. Comparison of PCR and RPA for High Multiplex Probes

### Probe Precursors

3912 probe precursors (average length 90 nt) (comprising 978 unique sequences; SEQ ID NO: 1-978) were synthesized on a programmable microarray from LC Sciences. 25 μL of nuclease-free water was added to the lypholised sample making the concentration 0.064 pmol/μL.

### Processing of Probe Precursors

### PCR:

PCR amplification was performed in a total volume of 200 μL, containing 0.05 pmol multiplex probe precursors (total amount), 200 μM dNTP's, 4 μM F-primer (SEQ ID NO: 979), 4 μM R-biotin-primer (SEQ ID NO: 980) (the sequence of the not-biotinylated primer is given in SEQ ID NO: 981), 10 units cloned Pfu DNA polymerase_AD in 1× Cloned Pfu reaction buffer_AD (Agilent). The following PCR program was used: 5 minutes at 95°, followed by twenty cycles of 30 sec at 95° C., 2 minutes at 55° C., eight minutes at 72° C., followed by 10 minutes at 72° C.

### RPA:

A Recombinase Polymerase Amplification (RPA) was performed using the TwistAmp Basic kit from TwistDX (order #TABAS01KIT). A reaction mix was prepared containing 0.05 pmol multiplex probe precursors (total amount), 700 nM F-primer (SEQ ID NO: 979), 700 nM R-biotin-primer (SEQ ID NO: 980) and 29.5 μL Rehydration Buffer. MQ was added to the reaction mixture to an end volume of 47.5 μL. After addition of 2 μL of 280 mM MgAc to start the reaction, the mixture was incubated for 40 minutes at 38° C.

The sample was purified with a QIAquick PCR Purification column according to manufacturer's protocol and using 50 μL EB buffer for elution.

### Results:

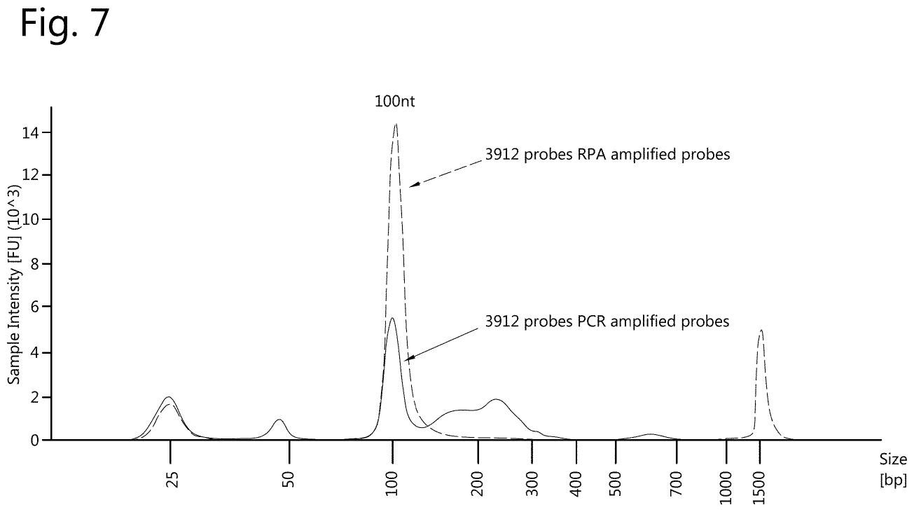

The quality and size of the amplicons produced via PCR and RPA, respectively, was checked on the Tapestation with a Agilent D1000 screen tape (FIG. 7). PCR resulted in a low specific amplicon yield (as compared to RPA), which is likely due to hetero-duplex formation.

### Example 2. Method for Probe Amplification and Purification

### Probe Precursors

3912 probe precursors (average length 90 nt) (comprising 978 unique sequences; SEQ ID NO: 1-978) were synthesized on a programmable microarray from LC Sciences. 25 μL of nuclease-free water was added to the lyophilized sample making the concentration 0.064 pmol/μL.

Processing of Probe Precursors A Recombinase Polymerase Amplification (RPA) was performed using the TwistAmp Basic kit from TwistDX (order #TABAS01KIT). A single RPA reaction mix was prepared containing 0.01 pmol multiplex probe precursors (total amount), 700 nM F-primer (SEQ ID NO: 979), 700 nM R-biotin-primer (SEQ ID NO: 980) and 29.5 μL Rehydration Buffer. MQ was added to the reaction mixture to an end volume of 47.5 μL. This reaction mix was added to the freeze-dried Basic reaction. After addition of 2 μL of 280 mM MgAc to start the reaction, the mixture was incubated for 40 minutes at 38° C.

Eight separate RPA reactions were performed and pooled. The amplicons were purified using two QIAquick PCR Purification columns according to manufacturer's protocol and using 50 μL EB buffer per column for elution, i.e. 100 μL EB buffer total.

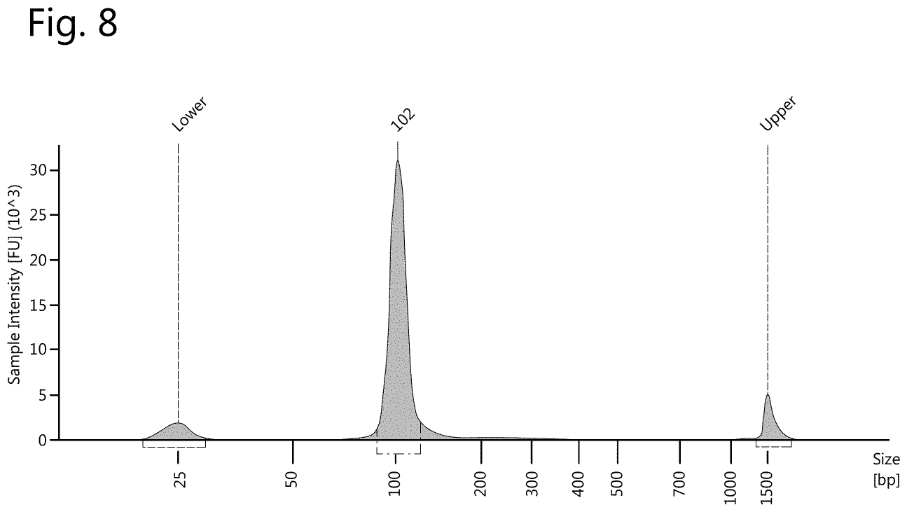

The quality and size of the amplicons was checked on the Tapestation with an Agilent D1000 screen tape (FIG. 8). The concentration was measured with the Qubit dsDNA BR Assay Kit (cat #Q32850) from Life Technologies (Table 1). The total yield is about 8 μg amplicons.

TABLE 1
Result Qubit (1 μL of 100 μL total was checked)

| pmol | # RPA | Conc Qubit | Total volume | Total yield | Yield per |
|---|---|---|---|---|---|
| input | reactions | (ng/μL) | (μL) | (μg) | RPA (μg) |
| 0.01 | 8 | 86.2 | 93 | 8.0 | 1.0 |

Nicking of Single Stranded 55-63 nt. Targeting Probes

The flanking sequences of the (85-93 nt.) probe precursors contained recognition sites for nicking restriction endonucleases at the junctions with the targeting arms.

Two nicking reactions were performed as follows: 50 μL column-purified RPA reaction, 10 μL 10× Cut-Smart buffer (New England Biolabs), 5 μL Nt.Alwl (10 U/μL, New England Biolabs) and 35 μL MQ were mixed and incubated at 37° C. for two hours. After this step, 5 μL of NbBsrDI (10 U/μL, New England Biolabs) was added and incubated at 65° C. for two hours followed by an inactivating step of 20 minutes at 80° C.

The nicked RPA product of two reactions was pooled and purified with two QIAquick PCR Purification columns according to manufacturer's protocol, the elution was done in 80 μL EB buffer per column (160 μL total).

Purification with Biotin

Dynabeads MyOne Streptavidin Cl (cat #65002) were used for immobilization of the QIAquick purified nicked RPA product according to manufacturer's protocol. The 160 μL QIAquick purified product was split in three aliquots of 53.3 μL. To each of these aliquots, an amount 200 μL of beads was added. Incubation was performed and washing was performed according to manufacturer's protocol. In a final step, the beads were re-suspended in 20 μL EB buffer per aliquot.

Release of Single Stranded 55-63 nt. Targeting Probes

Each of the three aliquots obtained above were subjected to chemical denaturation. To perform a chemical denaturation, NaOH was added to an end concentration of 0.9 M. The mixture was incubated for 10 minutes at room temperature and then placed on a magnet. The supernatant was taken and neutralized by adding HCl in an equimolar amount as NaOH added.

The supernatants of the three aliquots were pooled and purified with the ssDNA/RNA Clean & Concentrator from ZYMO RESEARCH (Cat #D7010) according to manufacturer's protocol. The elution was done in 40 μL EB (Qiagen).

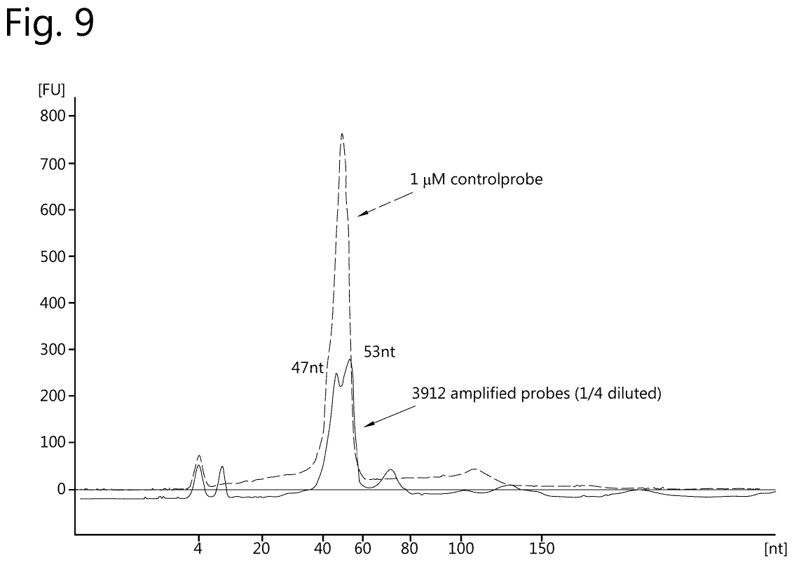

The quality and size of the probes was checked on the Bioanalyzer with an Agilent Small RNA kit using an ordered probe set of comparable length (54-68 nts) as positive control (FIG. 9). The concentration was measured with the Qubit ssDNA Assay Kit (cat #Q10212) from Life Technologies (Table 2).

TABLE 2
Result Qubit

| Start amount of precursor probe | | | | | | |
|---|---|---|---|---|---|---|
| | | Total | | | | Net fold |
| Per RPA | # | start | Amount of created probe | | | increase |
| reaction | RPA | amount | | Conc | Yield per | probe |
| (pmol) | reactions | (pmol) | (μL) | (pmol/μL) | RPA (μg) | yield |
| 0.01 | 8 | 0.08 | 40 | 1.1 | 44 | 550 |

### Results

The present probe amplification method resulted in a high net probe yield (net fold increase of probe yield of 550) achieved with a very low amount of input material (0.01 pmol). This method allows for amplification of oligonucleotides at a high multiplex level without creating hetero-duplex molecules. The use of biotin beads for purification renders a very fast and easy method. Further, the chemical denaturation and neutralization for a release of the amplified oligonucleotides is very efficient, whereas using heat for denaturation and release does not yield a detectable amount of products.

### Example 3. Parameter Variation

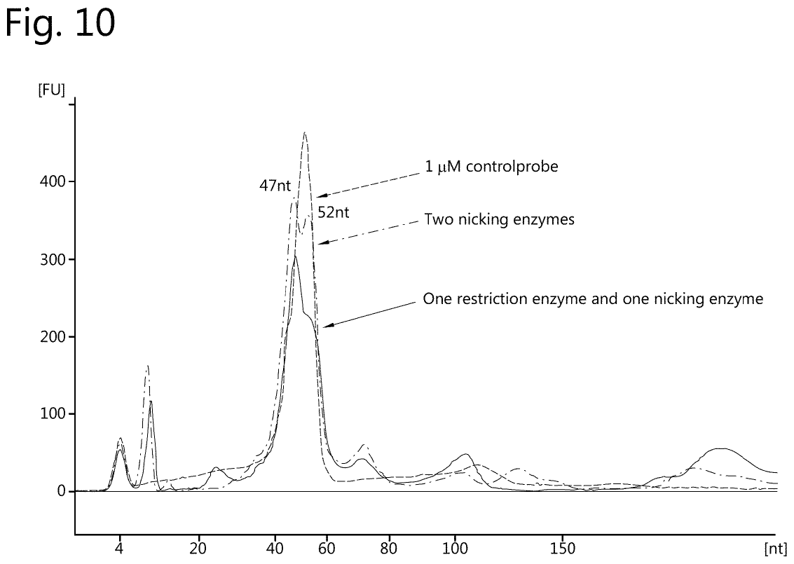

In set of comparative experiments, the method described in detail in Example 2 was performed while varying one parameter at the time. The experiments were designed as follows:
1. Method as detailed in Example 2, but with 2.5 μL of each nicking enzyme (12.5 units each) instead of 5 μL (50 units each) as done in Example 2 (FIG. 10 “Two nicking enzymes”).
2. Method as detailed in Example 2, wherein the nicking enzyme Nt.Alwl is replaced with a Alwl (New England Biolabs), at the same volume and units as indicated under 1 (FIG. 10: “One restriction enzyme and one nicking enzyme”).

The quality and size of the probes was checked on the Bioanalyzer with an Agilent Small RNA kit (FIG. 10). Replacing the first nicking enzyme with a restriction enzyme resulted in comparable yield.

The skilled person understands that although the experiments specified herein concern oligonucleotide for use as probes, the same protocol applies to oligonucleotides intended for a different use.

### Example 4. Amplified Oligonucleotide Probe Validation

The 3912 oligonucleotide probes produced in using the method as detailed in Example 2 were designed to detect 326 different SNPs in the maize genome (Zea mays), each having 2 alleles (i.e. 326-plex), in an OLA assay. The probes as produced in Example 2 where validated by testing them in OLA assays for genotyping 5 different genomic maize DNA samples, prepared from an F2 Zea mays mapping population. More in particular, reproducibility of OLA assays using these probes was tested by comparing the genotype calling between duplicates of each of the 5 different genomic maize DNA samples. Further, OLA assays using these probes were validated by comparing the genotype calling within these 5 different samples to genotype calling using the same OLA assay and the same 5 different genomic maize DNA samples, wherein the probes are replaced by individually synthesized probes of an existing 1056-plex OLA assay (IDT, Integrated DNA Technologies), which comprises the 326-plex probes for detecting the SNP alleles of the 326 loci.

The oligonucleotide probes (5′-3′ orientation) were designed using common procedures based on the known sequence of the loci and selected to discriminate the SNP alleles for each of the 326 loci. PCR primer binding regions, locus and allele identifiers were included. More in particular, the reverse complement of a first primer binding sequence (having a length of 16 nucleotides) is located at the 5′ end of the allele specific probe, and a second primer binding sequence (having a length of 18 nucleotides) is located at the 3′ end of the locus specific probe. Adjacent to the 3′ end of the first primer binding sequence are the following elements (in the 5′ to 3′ direction): a universal sequence of 13 nucleotides, a 4-base allele identifier is located, and a first target specific sequence. Adjacent to the 5′ end of the second primer sequence are the following elements (in the 3′ to 5′ direction): a universal sequence of 14 nucleotides, an 8-base locus identifier is located, and a second target specific sequence.

Below, the procedure of an OLA assay is described using probes as prepared in Example 2. The whole procedure is performed identically for individually synthesized probes, wherein the 1 μL 326-plex-probe mix as produced in Example 2 (3.4 nM per locus; 1.12 μM total) in the ligation reaction, is replaced by 1 μL 1056-plex-probe mix ordered from IDT and subsequently phosphorylated (0.4 nM per locus; 0.4 μM in total).

### OLA Assay Procedure

Ligation reactions were prepared as follows: 100 to 200 ng genomic DNA in 5 μL was combined with 1 μl 10× Taq DNA Ligase Buffer (200 mM Tris-HCl pH 7.6, 250 mM KAc, 100 mM MgAc, 10 mM NAD, 100 mM Dithiothreitol, 1% Triton-X100), 4 units Taq DNA ligase (New England BioLabs), 1 μl 326-plex-probe mix as produced in Example 2 (3.4 nM per locus; 1.12 μM total) or 1 μL 1056-plex-probe mix ordered from LC Sciences and subsequently phosphorylated (0.4 nM per locus; 0.4 μM in total) and MilliQ water to a total of 10 μl. Ligation reactions were setup in quadruplicate per genomic DNA sample. The reaction mixtures was incubated for 1 minute and 30 seconds at 94° C. followed by a temperature decrease of 1.0° C. per 30 seconds until 60° C., followed by an incubation at 60° C. for approximately 18 hours. Reactions were kept at 4° C. until further use. Ligation reactions were 4× diluted with MilliQ water.

Amplification of the ligation products was performed using a first and second amplification primer. The first amplification primer is designed to comprise at its 3′ terminus a sequence (16 nucleotides) for annealing to the first primer binding sequence, a P7 sequence located at its 5′ terminus, and in between these elements a 5-base sample identifier. The second primer is designed to comprise at its 3′ terminus a sequence (18 nucleotides) for annealing to the second primer binding sequence, a P5 sequence located at its 5′ terminus, and in between these elements a 6-base plate identifier.

Amplification of the ligation products was carried out in the following reaction mixture: 10 μl 4× diluted ligation reaction, 0.05 μM (end concentration) of each primer (first and second amplification primer), 20 μL of Phusion Hot Start FLX master mix (Bioké) and MilliQ water to a total of 40 μl. Each ligation product was amplified three times; per 5 different genomic DNA samples, in total 60 PCR reaction were performed. The thermocycling profile was performed on a PE9700 (Perkin Elmer Corp.) with a gold or silver block using the following conditions: Step 1: Pre PCR incubation: 30 seconds at 98° C. Step 2: Denaturation: 10 seconds at 98° C.; Annealing: 15 seconds at 65° C. Extension: 15 seconds at 72° C. Total cycle number was 29. Step 3: Extension 5 minutes at 72° C. Reactions were kept at 4° C. until further use. Amplification products of the in total 60 PCR reactions were pooled (60×40 μl) and purified using two PCR purification columns (Qiagen) and eluted in 15 μl MilliQ water per column, 30 μL total.

Purification of the amplicons was done with a Pippin Prep of Sage Science. Four times 900 ng was purified using a 3% cassette and marker C with no overflow. The range 170 bp until 230 bp was eluted. The eluted product were purified using the Minelute kit (Qiagen) and eluted in 15 μL.

Sequencing of the amplicons was performed using an Illumina MiSeq nano run. Resulting sequencing data was de-multiplexed in which reads are assigned to each of the samples used. Data of two quadruplicates per sample of genomic DNA were pooled for sufficient genomic coverage needed for efficient genotyping and further processed and considered as a singlet, thereby resulting in a duplicate result per sample of genomic DNA.

### Results

For the total of 5 samples (comprising a total theoretical number of 5×326=1630 genotypes), a total of 1452 genotypes were called, with a reproducibility between duplicates of 99.8%, i.e. 99.8% of the genotypes called using a 326-plex assay with probes produced in Example 2 are identical between the duplicates. When using the individually synthesized probes, a total of 1452 genotypes were called, which were 97.5% identical to the genotypes called using the probes produced in Example 2.

TABLE X
Performance of 326-plex OLA assays using of 5 maize genomic DNA
samples (total theoretical number of genotypes being 1630)

| | # genotypes | call | | |
|---|---|---|---|---|
| Probes | called | rate | Validity¹⁾ | Reproducibility²⁾ |
| Individually | 1452 | 89.1% | | |
| synthesized | | | | |
| Prepared | 1449 | 88.9% | 97.5% | 99.8% |
| according | | | | |
| to Example 2 | | | | |

¹⁾Percentage of called genotypes matching called genotypes in the OLA assay using individually synthesized probes.
²⁾Percentage of called genotypes matching between duplicates.

1. A method for producing one or more single-stranded oligonucleotides having a sequence of interest, wherein the method comprises:
(a) providing at least one single- or double-stranded nucleic acid precursor comprising a first strand and optionally a second strand that is complementary to the first strand, wherein the first strand comprises the following elements in a 5′ to 3′ direction:
(i) a first primer binding site;
(ii) a first endonuclease recognition site;
(iii) the sequence of interest;
(iv) a second endonuclease recognition site; and,
(v) a second primer binding site;
wherein the first endonuclease recognition site is designed such that, after duplexing, a first endonuclease cleaves the sugar-phosphate backbone of the first strand immediately upstream of the sequence of interest; and,
wherein the second endonuclease recognition site is designed such that, after duplexing, a second endonuclease cleaves the sugar-phosphate backbone of the first strand immediately downstream of the sequence of interest;
(b) amplifying the precursor of (a) by an amplification method, using a first primer capable of hybridizing to the first primer binding site and a second primer capable of hybridizing to the second primer binding site, wherein the second primer comprises an affinity-tag that is not present on the first primer, to produce an amplified double-stranded nucleic acid precursor comprising the tag;
(c) digesting the amplified double-stranded precursor obtained in (b) with the first and the second endonuclease to produce an amplified double-stranded nucleic acid precursor with cleavages of the sugar-phosphate backbone immediately up- and downstream of the sequence of interest and with an intact sugar-phosphate backbone between the tag up to and including the sequence complementary to the sequence of interest;
(d) immobilizing the amplified double-stranded nucleic acid precursor on a solid support by affinity capture of the tagged complementary second strand;
(e) denaturing the amplified double-stranded precursor, thereby releasing the single-stranded oligonucleotide having the sequence of interest; and
(f) removing the solid support to obtain the single-stranded oligonucleotide having the sequence of interest.

2. The method according to claim 1, wherein steps (c) and (d) are reversed or wherein steps (d) and (e) are reversed.

3. The method according to claim 1, further comprising (g) purifying the single-stranded oligonucleotide.

4. The method according to claim 1, wherein the denaturing in (e) comprises chemical denaturing.

5. The method according to claim 4, wherein the chemical denaturing is by increasing the pH by the addition of an alkali hydroxide at a concentration of about 0.5-1.5 M.

6. The method according to claim 1, wherein the nucleic acid precursor consists of 20-200 nucleotides.

7. The method according to claim 6, wherein the nucleic acid precursor has a sequence selected from the group consisting of SEQ ID NO: 1-SEQ ID NO: 978.

8. The method according to claim 1, wherein the sequence of interest is at least partly complementary to a predetermined genomic sequence.

9. The method according to claim 1, wherein the produced oligonucleotide is suitable for use in a multiplex OLA assay, hybridization assay, or a multiplex oligonucleotide-based amplification assay.

10. The method according to claim 1, wherein the nucleic acid precursor is a single-stranded nucleic acid precursor.

11. The method according to claim 1, wherein the amplification method in (b) is an isothermal amplification method.

12. The method according to claim 11, wherein the isothermal amplification method is Recombinase Polymerase Amplification (RPA) or Helicase Dependent Amplification (HDA).

13. The method according to claim 1, wherein the first and the second endonuclease are two different enzymes.

14. The method according to claim 1, wherein the first endonuclease in (c) cleaves:
(i) the first DNA strand; or
(ii) the first and the second DNA strand.

15. The method according to claim 1, wherein the amplified double-stranded precursor from (b) is purified prior to binding the solid support in (d).

16. The method according to claim 1, wherein the tag is biotin and the solid support comprises streptavidin.

17. The method according to claim 9, wherein in (a) two or more nucleic acid precursors are provided that have a distinct sequence of interest.

18. The method according to claim 1, wherein the first primer can selectively anneal to only the first primer binding site and a second primer can selectively anneal to only the second primer binding site.

19. The method according to claim 1, wherein the sequence of interest does not comprise the first and the second endonuclease recognition sites or reverse complement thereof.

20. A single or a double-stranded nucleic acid precursor comprising a first strand and optionally a second strand that is complementary to the first strand, wherein the first strand comprises the following elements in a 5′ to 3′ direction:
(i) a first primer binding site;
(ii) a first endonuclease recognition site;
(iii) the sequence of interest;
(iv) a second endonuclease recognition site; and,
(v) a second primer binding sequence;
wherein the first endonuclease recognition site is designed such that, after duplexing, a first endonuclease cleaves the sugar-phosphate backbone of the first strand immediately upstream of the sequence of interest; and,
wherein the second endonuclease recognition site is designed such that, after duplexing, a second endonuclease cleaves the sugar-phosphate backbone of the first strand immediately downstream of the sequence of interest.

21. The method according to claim 20, wherein a first primer can selectively anneal to only the first primer binding site and a second primer can selectively anneal to only the second primer binding site.

22. The method according to claim 20, wherein the sequence of interest does not comprise the first and the second endonuclease recognition sites or reverse complement thereof.

23. A double-stranded nucleic acid precursor according to claim 20, wherein the precursor further comprises an affinity tag located at the 5′ end of the second strand, wherein preferably the affinity tag is only at the 5′ end of the second strand.

24. A solid support comprising the double-stranded nucleic acid precursor as defined in claim 20, bound to the solid support by means of affinity-capture.

25. A kit of parts, comprising:
(a) a container comprising the second endonuclease and optionally the first endonuclease;
(b) a container comprising enzymes for use in amplification step b), optionally in combination with the first and/or tagged second primer;
(c) a container comprising a solid support for affinity purification; and optionally
(d) a container comprising a chemical for denaturation.
